SEQUENCE LISTING (135 sequences)

SEQ ID NO: 1 - protein, 267 aa, Artificial sequence
  Synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 2 - protein, 258 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 3 - protein, 268 aa, Artificial sequence
  Synthetic construct
MVLLVTSLLLCELPHPAFLLIPDTEVQLVESGGGLVKPGGSLKLSCAASGFAFSIYDMSWVRQTPEKRLEWVAYISSGGGTYYPDTVKGRFTISRDNAKNTLYLQMSSLKSEDTAMYYCARHSGYGTHWGVLFAYWGQGTLVTVSAGGGGSGGGGSGGGGSDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIK

SEQ ID NO: 4 - protein, 503 aa, Artificial sequence
  Synthetic construct
MLLLVTSLLLCELPHPAFLLIPDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 5 - protein, 72 aa, Artificial sequence
  Synthetic construct
AAATTTPAPRPPTPAPTIASQPLSLRPEACRPAAGGAVHTRGLDFACDIYIWAPLAGTCGVLLLSLVITLYC

SEQ ID NO: 6 - protein, 69 aa, Artificial sequence
  Synthetic construct
AAAIEVMYPPPYLDNEKSNGTIIHVKGKHLCPSPLFPGPSKPFWVLVVVGGVLACYSLLVTVAFIIFWV

SEQ ID NO: 7 - protein, 68 aa, Artificial sequence
  Synthetic construct
AAAVITHKWTTSLSAKFKCTAGNKVSKESSVEPVSCPEKGLDIYLIIGICGGGSLLMVFVALLVFYIT

SEQ ID NO: 8 - protein, 116 aa, Artificial sequence
  Synthetic construct
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 9 - protein, 112 aa, Artificial sequence
  Synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 42 aa, Artificial sequence
  Synthetic construct
KRGRKKLLYIFKQPFMRPVQTTQEEDGCSCRFPEEEEGGCEL

SEQ ID NO: 11 - protein, 42 aa, Artificial sequence
  Synthetic construct
VRSKRSRLLHSDYMNMTPRRPGPTRKHYQPYAPPRDFAAYRS

SEQ ID NO: 12 - protein, 567 aa, Artificial sequence
  Synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 609 aa, Artificial sequence
  Synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 609 aa, Artificial sequence
  Synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 609 aa, Artificial sequence
  Synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 558 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 600 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 600 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 600 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 452 aa, Artificial sequence
  Synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 21 - protein, 455 aa, Artificial sequence
  Synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 22 - protein, 451 aa, Artificial sequence
  Synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 23 - protein, 443 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAIEVMYPPPYLDNEKSNGTIIHVKGKHLCPSPLFPGPSKPFWVLVVVGGVLACYSLLVTVAFIIFWVKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 24 - protein, 446 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 25 - protein, 453 aa, Artificial sequence
  Synthetic construct
MVLLVTSLLLCELPHPAFLLIPDTEVQLVESGGGLVKPGGSLKLSCAASGFAFSIYDMSWVRQTPEKRLEWVAYISSGGGTYYPDTVKGRFTISRDNAKNTLYLQMSSLKSEDTAMYYCARHSGYGTHWGVLFAYWGQGTLVTVSAGGGGSGGGGSGGGGSDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKAAAIEVMYPPPYLDNEKSNGTIIHVKGKHLCPSPLFPGPSKPFWVLVVVGGVLACYSLLVTVAFIIFWVKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 26 - protein, 456 aa, Artificial sequence
  Synthetic construct
MVLLVTSLLLCELPHPAFLLIPDTEVQLVESGGGLVKPGGSLKLSCAASGFAFSIYDMSWVRQTPEKRLEWVAYISSGGGTYYPDTVKGRFTISRDNAKNTLYLQMSSLKSEDTAMYYCARHSGYGTHWGVLFAYWGQGTLVTVSAGGGGSGGGGSGGGGSDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKAAATTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 27 - protein, 452 aa, Artificial sequence
  Synthetic construct
MVLLVTSLLLCELPHPAFLLIPDTEVQLVESGGGLVKPGGSLKLSCAASGFAFSIYDMSWVRQTPEKRLEWVAYISSGGGTYYPDTVKGRFTISRDNAKNTLYLQMSSLKSEDTAMYYCARHSGYGTHWGVLFAYWGQGTLVTVSAGGGGSGGGGSGGGGSDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKAAAVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 28 - protein, 688 aa, Artificial sequence
  Synthetic construct
MLLLVTSLLLCELPHPAFLLIPDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKAAAIEVMYPPPYLDNEKSNGTIIHVKGKHLCPSPLFPGPSKPFWVLVVVGGVLACYSLLVTVAFIIFWVKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 29 - protein, 691 aa, Artificial sequence
  Synthetic construct
MLLLVTSLLLCELPHPAFLLIPDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKAAATTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 30 - DNA, 801 bp, Artificial sequence
  Synthetic construct
atgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggcagcggcaagcctggatctggcgagggaagcaccaagggcgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtcatct

SEQ ID NO: 31 - DNA, 774 bp, Artificial sequence
  Synthetic construct
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaa

SEQ ID NO: 32 - DNA, 804 bp, Artificial sequence
  Synthetic construct
atggttttattggtcacatcactgctcctctgtgaactgcctcatcccgcttttttattgattcccgacactgaagtccagctcgtggaatctggagggggcctggtgaaacctgggggatctctcaaactgtcttgtgccgcttctggctttgcttttagcatctacgacatgtcctgggtccggcagacacctgaaaaacgcctggagtgggtcgcctacatttctagtgggggcggaacatactaccccgataccgtgaagggacgctttacaatttctagggataacgccaaaaacaccctgtacctccagatgtcatccctgaaatctgaggatactgccatgtactactgtgctaggcattctggctacggaacacattggggagtgctcttcgcttactggggccaggggactctcgtcactgtctctgctggcgggggaggctctggcggaggcggttccggaggcggagggagtgatattcagatgactcagaccacctcttctctgtccgcttctctgggcgatagagtgacaatctcctgtcgggcatcacaggatattagcaattacctgaactggtaccagcagaaacccgatggaaccgtcaaactgctcatctactacacctccatcctccactctggcgtgccatctcgattttctggatctggctctggaaccgactactctctcacaatctccaacctggaacaggaggattttgccacctacttttgtcagcagggcaatactctgccttggacctttgggggcggaaccaaactggaaatcaag

SEQ ID NO: 33 - DNA, 1509 bp, Artificial sequence
  Synthetic construct
atgctgctgctggtcacatctctgctgctgtgcgaactgccccatcctgcctttctgctgatccccgatatcgtgctgacacagagccctgccattctgtctgctagccctggcgagaaagtgaccatgacctgtagagccagcagctccgtgaactacatggactggtatcagaagaagcccggcagcagccccaagccttggatctacgctacaagcaatctggccagcggcgtgccagccagattttctggttctggcagcggcaccagctacagcctgacaatctctagagtggaagccgaggacgccgccacctactattgccagcagtggtccttcaatcctcctacctttggcggaggcaccaagctggaaatcaagggctctacatctggcggcggttctggtggtggaagcggaggcggaggatcttctgaagttcagctgcagcagtctggcgccgaacttgtgaaacctggcgcctctgtgaagatgagctgcaaggcctctggctacaccttcacaagctacaacatgcactgggtcaagcagacccctggacagggactcgaatggatcggagccatctatcccggcaacggcgacacctcctacaaccagaagttcaagggcaaagccacactgaccgccgacaagagcagctctacagcctacatgcagctgagcagcctgaccagcgaggacagcgccgattactactgcgccagaagcaactactacggcagctcctactggttcttcgatgtgtggggagccggcaccacagtgacagtgtctagcgagcctaagagctgcgacaagacccacacctgtcctccatgtcctgctcctccagtggccggaccttccgtgtttctgttccctccaaagcctaaggacaccctgatgatcagcaggacccctgaagtgacctgtgtggtggtcgatgtgtcccacgaggacccagaagtgaagttcaattggtacgtggacggcgtggaagtgcacaacgccaagaccaagcctagagaggaacagtaccagagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttctcctgttctgtgatgcacgaggccctgcacaaccactacacccagaaaagcctgtctctgagccccggcaagaaggaccctaaa

SEQ ID NO: 34 - DNA, 216 bp, Artificial sequence
  Synthetic construct
gcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgc

SEQ ID NO: 35 - DNA, 207 bp, Artificial sequence
  Synthetic construct
gcggccgccattgaggtcatgtacccaccaccttacctcgacaacgaaaaatcaaacgggacgattattcacgtcaaaggcaagcatctgtgcccgtcacctctgttccccggaccaagcaaaccgttctgggtgcttgttgtcgtcggcggggtccttgcttgttactcacttctggttaccgttgcttttatcattttttgggtg

SEQ ID NO: 36 - DNA, 204 bp, Artificial sequence
  Synthetic construct
gcggccgccgtgattacacacaagtggacaacaagcctgagcgccaagttcaagtgcaccgccggcaacaaggtgtccaaagaaagcagcgtggaacccgtgtcttgccccgagaaaggcctggacatctacctgatcatcggcatctgtggcggcggaagcctgctgatggtttttgtggccctgctggtgttctacatcacc

SEQ ID NO: 37 - DNA, 348 bp, Artificial sequence
  Synthetic construct
aagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaac

SEQ ID NO: 38 - DNA, 339 bp, Artificial sequence
  Synthetic construct
agagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataa

SEQ ID NO: 39 - DNA, 126 bp, Artificial sequence
  Synthetic construct
aagcggggcagaaagaagctgctgtacatcttcaagcagcccttcatgcggcccgtgcagaccacacaagaggaagatggctgctcctgcagattccccgaggaagaagaaggcggctgcgagctg

SEQ ID NO: 40 - DNA, 126 bp, Artificial sequence
  Synthetic construct
gtgcggagcaagagaagcagactgctgcacagcgactacatgaacatgacccctagacggcccggacctaccagaaagcactaccagccttacgctcctcctcgggactttgccgcctatcggagc

SEQ ID NO: 41 - DNA, 1704 bp, Artificial sequence
  Synthetic construct
atgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggcagcggcaagcctggatctggcgagggaagcaccaagggcgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtcatctgcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaacagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataa

SEQ ID NO: 42 - DNA, 1830 bp, Artificial sequence
  Synthetic construct
atgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggcagcggcaagcctggatctggcgagggaagcaccaagggcgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtcatctgcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaacaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataa

SEQ ID NO: 43 - DNA, 1830 bp, Artificial sequence
  Synthetic construct
atgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggcagcggcaagcctggatctggcgagggaagcaccaagggcgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtcatctgcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaacagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataa

SEQ ID NO: 44 - DNA, 1830 bp, Artificial sequence
  Synthetic construct
atgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggcagcggcaagcctggatctggcgagggaagcaccaagggcgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtcatctgcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaacgtgcggagcaagagaagcagactgctgcacagcgactacatgaacatgacccctagacggcccggacctaccagaaagcactaccagccttacgctcctcctcgggactttgccgcctatcggagcagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataa

SEQ ID NO: 45 - DNA, 1677 bp, Artificial sequence
  Synthetic construct
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaacagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataa

SEQ ID NO: 46 - DNA, 1803 bp, Artificial sequence
  Synthetic construct
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaacaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataa

SEQ ID NO: 47 - DNA, 1803 bp, Artificial sequence
  Synthetic construct
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaacagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataa

SEQ ID NO: 48 - DNA, 1803 bp, Artificial sequence
  Synthetic construct
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaacgtgcggagcaagagaagcagactgctgcacagcgactacatgaacatgacccctagacggcccggacctaccagaaagcactaccagccttacgctcctcctcgggactttgccgcctatcggagcagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataa

SEQ ID NO: 49 - DNA, 1356 bp, Artificial sequence
  Synthetic construct
atgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggcagcggcaagcctggatctggcgagggaagcaccaagggcgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtcatctgcggccgccattgaggtcatgtacccaccaccttacctcgacaacgaaaaatcaaacgggacgattattcacgtcaaaggcaagcatctgtgcccgtcacctctgttccccggaccaagcaaaccgttctgggtgcttgttgtcgtcggcggggtccttgcttgttactcacttctggttaccgttgcttttatcattttttgggtgaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaac

SEQ ID NO: 50 - DNA, 1365 bp, Artificial sequence
  Synthetic construct
atgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggcagcggcaagcctggatctggcgagggaagcaccaagggcgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtcatctgcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaac

SEQ ID NO: 51 - DNA, 1353 bp, Artificial sequence
  Synthetic construct
atgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggcagcggcaagcctggatctggcgagggaagcaccaagggcgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtcatctgcggccgccgtgattacacacaagtggacaacaagcctgagcgccaagttcaagtgcaccgccggcaacaaggtgtccaaagaaagcagcgtggaacccgtgtcttgccccgagaaaggcctggacatctacctgatcatcggcatctgtggcggcggaagcctgctgatggtttttgtggccctgctggtgttctacatcaccaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaac

SEQ ID NO: 52 - DNA, 1329 bp, Artificial sequence
  Synthetic construct
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgccattgaggtcatgtacccaccaccttacctcgacaacgaaaaatcaaacgggacgattattcacgtcaaaggcaagcatctgtgcccgtcacctctgttccccggaccaagcaaaccgttctgggtgcttgttgtcgtcggcggggtccttgcttgttactcacttctggttaccgttgcttttatcattttttgggtgaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaac

SEQ ID NO: 53 - DNA, 1338 bp, Artificial sequence
  Synthetic construct
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaac

SEQ ID NO: 54 - DNA, 1359 bp, Artificial sequence
  Synthetic construct
atggttttattggtcacatcactgctcctctgtgaactgcctcatcccgcttttttattgattcccgacactgaagtccagctcgtggaatctggagggggcctggtgaaacctgggggatctctcaaactgtcttgtgccgcttctggctttgcttttagcatctacgacatgtcctgggtccggcagacacctgaaaaacgcctggagtgggtcgcctacatttctagtgggggcggaacatactaccccgataccgtgaagggacgctttacaatttctagggataacgccaaaaacaccctgtacctccagatgtcatccctgaaatctgaggatactgccatgtactactgtgctaggcattctggctacggaacacattggggagtgctcttcgcttactggggccaggggactctcgtcactgtctctgctggcgggggaggctctggcggaggcggttccggaggcggagggagtgatattcagatgactcagaccacctcttctctgtccgcttctctgggcgatagagtgacaatctcctgtcgggcatcacaggatattagcaattacctgaactggtaccagcagaaacccgatggaaccgtcaaactgctcatctactacacctccatcctccactctggcgtgccatctcgattttctggatctggctctggaaccgactactctctcacaatctccaacctggaacaggaggattttgccacctacttttgtcagcagggcaatactctgccttggacctttgggggcggaaccaaactggaaatcaaggcggccgccattgaggtcatgtacccaccaccttacctcgacaacgaaaaatcaaacgggacgattattcacgtcaaaggcaagcatctgtgcccgtcacctctgttccccggaccaagcaaaccgttctgggtgcttgttgtcgtcggcggggtccttgcttgttactcacttctggttaccgttgcttttatcattttttgggtgaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaac

SEQ ID NO: 55 - DNA, 1368 bp, Artificial sequence
  Synthetic construct
atggttttattggtcacatcactgctcctctgtgaactgcctcatcccgcttttttattgattcccgacactgaagtccagctcgtggaatctggagggggcctggtgaaacctgggggatctctcaaactgtcttgtgccgcttctggctttgcttttagcatctacgacatgtcctgggtccggcagacacctgaaaaacgcctggagtgggtcgcctacatttctagtgggggcggaacatactaccccgataccgtgaagggacgctttacaatttctagggataacgccaaaaacaccctgtacctccagatgtcatccctgaaatctgaggatactgccatgtactactgtgctaggcattctggctacggaacacattggggagtgctcttcgcttactggggccaggggactctcgtcactgtctctgctggcgggggaggctctggcggaggcggttccggaggcggagggagtgatattcagatgactcagaccacctcttctctgtccgcttctctgggcgatagagtgacaatctcctgtcgggcatcacaggatattagcaattacctgaactggtaccagcagaaacccgatggaaccgtcaaactgctcatctactacacctccatcctccactctggcgtgccatctcgattttctggatctggctctggaaccgactactctctcacaatctccaacctggaacaggaggattttgccacctacttttgtcagcagggcaatactctgccttggacctttgggggcggaaccaaactggaaatcaaggcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaac

SEQ ID NO: 56 - DNA, 1356 bp, Artificial sequence
  Synthetic construct
atggttttattggtcacatcactgctcctctgtgaactgcctcatcccgcttttttattgattcccgacactgaagtccagctcgtggaatctggagggggcctggtgaaacctgggggatctctcaaactgtcttgtgccgcttctggctttgcttttagcatctacgacatgtcctgggtccggcagacacctgaaaaacgcctggagtgggtcgcctacatttctagtgggggcggaacatactaccccgataccgtgaagggacgctttacaatttctagggataacgccaaaaacaccctgtacctccagatgtcatccctgaaatctgaggatactgccatgtactactgtgctaggcattctggctacggaacacattggggagtgctcttcgcttactggggccaggggactctcgtcactgtctctgctggcgggggaggctctggcggaggcggttccggaggcggagggagtgatattcagatgactcagaccacctcttctctgtccgcttctctgggcgatagagtgacaatctcctgtcgggcatcacaggatattagcaattacctgaactggtaccagcagaaacccgatggaaccgtcaaactgctcatctactacacctccatcctccactctggcgtgccatctcgattttctggatctggctctggaaccgactactctctcacaatctccaacctggaacaggaggattttgccacctacttttgtcagcagggcaatactctgccttggacctttgggggcggaaccaaactggaaatcaaggcggccgccgtgattacacacaagtggacaacaagcctgagcgccaagttcaagtgcaccgccggcaacaaggtgtccaaagaaagcagcgtggaacccgtgtcttgccccgagaaaggcctggacatctacctgatcatcggcatctgtggcggcggaagcctgctgatggtttttgtggccctgctggtgttctacatcaccaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaac

SEQ ID NO: 57 - DNA, 2064 bp, Artificial sequence
  Synthetic construct
atgctgctgctggtcacatctctgctgctgtgcgaactgccccatcctgcctttctgctgatccccgatatcgtgctgacacagagccctgccattctgtctgctagccctggcgagaaagtgaccatgacctgtagagccagcagctccgtgaactacatggactggtatcagaagaagcccggcagcagccccaagccttggatctacgctacaagcaatctggccagcggcgtgccagccagattttctggttctggcagcggcaccagctacagcctgacaatctctagagtggaagccgaggacgccgccacctactattgccagcagtggtccttcaatcctcctacctttggcggaggcaccaagctggaaatcaagggctctacatctggcggcggttctggtggtggaagcggaggcggaggatcttctgaagttcagctgcagcagtctggcgccgaacttgtgaaacctggcgcctctgtgaagatgagctgcaaggcctctggctacaccttcacaagctacaacatgcactgggtcaagcagacccctggacagggactcgaatggatcggagccatctatcccggcaacggcgacacctcctacaaccagaagttcaagggcaaagccacactgaccgccgacaagagcagctctacagcctacatgcagctgagcagcctgaccagcgaggacagcgccgattactactgcgccagaagcaactactacggcagctcctactggttcttcgatgtgtggggagccggcaccacagtgacagtgtctagcgagcctaagagctgcgacaagacccacacctgtcctccatgtcctgctcctccagtggccggaccttccgtgtttctgttccctccaaagcctaaggacaccctgatgatcagcaggacccctgaagtgacctgtgtggtggtcgatgtgtcccacgaggacccagaagtgaagttcaattggtacgtggacggcgtggaagtgcacaacgccaagaccaagcctagagaggaacagtaccagagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttctcctgttctgtgatgcacgaggccctgcacaaccactacacccagaaaagcctgtctctgagccccggcaagaaggaccctaaagcggccgccattgaggtcatgtacccaccaccttacctcgacaacgaaaaatcaaacgggacgattattcacgtcaaaggcaagcatctgtgcccgtcacctctgttccccggaccaagcaaaccgttctgggtgcttgttgtcgtcggcggggtccttgcttgttactcacttctggttaccgttgcttttatcattttttgggtgaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaac

SEQ ID NO: 58 - DNA, 2073 bp, Artificial sequence
  Synthetic construct
atgctgctgctggtcacatctctgctgctgtgcgaactgccccatcctgcctttctgctgatccccgatatcgtgctgacacagagccctgccattctgtctgctagccctggcgagaaagtgaccatgacctgtagagccagcagctccgtgaactacatggactggtatcagaagaagcccggcagcagccccaagccttggatctacgctacaagcaatctggccagcggcgtgccagccagattttctggttctggcagcggcaccagctacagcctgacaatctctagagtggaagccgaggacgccgccacctactattgccagcagtggtccttcaatcctcctacctttggcggaggcaccaagctggaaatcaagggctctacatctggcggcggttctggtggtggaagcggaggcggaggatcttctgaagttcagctgcagcagtctggcgccgaacttgtgaaacctggcgcctctgtgaagatgagctgcaaggcctctggctacaccttcacaagctacaacatgcactgggtcaagcagacccctggacagggactcgaatggatcggagccatctatcccggcaacggcgacacctcctacaaccagaagttcaagggcaaagccacactgaccgccgacaagagcagctctacagcctacatgcagctgagcagcctgaccagcgaggacagcgccgattactactgcgccagaagcaactactacggcagctcctactggttcttcgatgtgtggggagccggcaccacagtgacagtgtctagcgagcctaagagctgcgacaagacccacacctgtcctccatgtcctgctcctccagtggccggaccttccgtgtttctgttccctccaaagcctaaggacaccctgatgatcagcaggacccctgaagtgacctgtgtggtggtcgatgtgtcccacgaggacccagaagtgaagttcaattggtacgtggacggcgtggaagtgcacaacgccaagaccaagcctagagaggaacagtaccagagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttctcctgttctgtgatgcacgaggccctgcacaaccactacacccagaaaagcctgtctctgagccccggcaagaaggaccctaaagcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaac

SEQ ID NO: 59 - protein, 484 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - DNA, 1455 bp, Artificial sequence
  Synthetic construct
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataa

SEQ ID NO: 61 - protein, 484 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - DNA, 1455 bp, Artificial sequence
  Synthetic construct
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcgtgcggagcaagagaagcagactgctgcacagcgactacatgaacatgacccctagacggcccggacctaccagaaagcactaccagccttacgctcctcctcgggactttgccgcctatcggagcagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataa

SEQ ID NO: 63 - protein, 493 aa, Artificial sequence
  Synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - DNA, 1482 bp, Artificial sequence
  Synthetic construct
atgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggcagcggcaagcctggatctggcgagggaagcaccaagggcgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtcatctgcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcgtgcggagcaagagaagcagactgctgcacagcgactacatgaacatgacccctagacggcccggacctaccagaaagcactaccagccttacgctcctcctcgggactttgccgcctatcggagcagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataa

SEQ ID NO: 65 - protein, 493 aa, Artificial sequence
  Synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - DNA, 1482 bp, Artificial sequence
  Synthetic construct
atgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggcagcggcaagcctggatctggcgagggaagcaccaagggcgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtcatctgcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataa

SEQ ID NO: 67 - protein, 528 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 68 - DNA, 1803 bp, Artificial sequence
  Synthetic construct
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataaaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaac

SEQ ID NO: 69 - protein, 26 aa, Artificial sequence
  Synthetic construct
KRREFATNFSLLKQAGDVEENPGPLE

SEQ ID NO: 70 - DNA, 78 bp, Artificial sequence
  Synthetic construct
aagagaagagaattcgcaacaaacttctctctgctgaaacaagccggagatgtcgaagagaatcctggaccgctcgag

SEQ ID NO: 71 - protein, 960 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKRREFATNFSLLKQAGDVEENPGPLEMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 72 - DNA, 2883 bp, Artificial sequence
  Synthetic construct
atgctgctgctggtcactagtctgcttttgtgtgagctgccacaccctgcatttttgcttataccacaggtgcagttgcaacaatctggacctggtctcgtcaaaccttcccagacccttagcttgacgtgtgcgattagtggcgatagcgtcagtagcaacagtgcagcatggaactggataagacaatctcccagccgaggtttggaatggctggggcgcacgtactatcgatccaaatggtataatgactacgcagtttccgtaaagtcacggattactataaatccggacacatcaaaaaatcaattcagtctgcagctcaactctgtaacgccagaagataccgctgtctactattgtgcacgggaggtaactggtgaccttgaggacgcattcgatatatggggccaaggaacgatggtcaccgtatctagtggcggtggtggttctgatatacaaatgacgcaatccccctcttccctttctgcctcggtgggagatcgagtaaccattacgtgcagagcttctcagacgatctggagctacctgaattggtatcaacagagacctggtaaagcgccaaatcttcttatatacgctgccagcagcttgcagtcaggagttccgtccagattcagcggtagggggtctgggaccgattttacattgaccatcagtagtcttcaggctgaagattttgcaacatattactgccaacagtcctattctattcctcaaactttcggacagggaaccaaattggaaataaagcttactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaagcgagggaggaaaaagttgttatacatttttaagcagcctttcatgcgccctgttcagaccacgcaggaggaggacggatgctcttgtcgcttccctgaggaggaggagggcggctgcgagttgagggtcaaattttctcgatccgcggatgcgcctgcatataaacaagggcaaaatcaactctacaatgaacttaacttaggacgacgggaagaatatgatgtactcgataagcgccgaggacgcgatccggaaatgggtggcaaaccacggcgtaaaaatccacaagaggggttatataacgagttacaaaaagacaaaatggccgaagcatattccgaaatcggcatgaagggtgagagacgacgaggcaaaggacatgacggtctctatcaaggcctttcaactgccactaaggatacgtatgatgcacttcatatgcaagccttaccaccacgcaagagaagagaattcgcaacaaacttctctctgctgaaacaagccggagatgtcgaagagaatcctggaccgctcgagatgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggcagcggcaagcctggatctggcgagggaagcaccaagggcgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtcatctgcggccgccattgaggtcatgtacccaccaccttacctcgacaacgaaaaatcaaacgggacgattattcacgtcaaaggcaagcatctgtgcccgtcacctctgttccccggaccaagcaaaccgttctgggtgcttgttgtcgtcggcggggtccttgcttgttactcacttctggttaccgttgcttttatcattttttgggtgaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaactaa

SEQ ID NO: 73 - protein, 963 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKRREFATNFSLLKQAGDVEENPGPLEMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 74 - DNA, 2892 bp, Artificial sequence
  Synthetic construct
atgctgctgctggtcactagtctgcttttgtgtgagctgccacaccctgcatttttgcttataccacaggtgcagttgcaacaatctggacctggtctcgtcaaaccttcccagacccttagcttgacgtgtgcgattagtggcgatagcgtcagtagcaacagtgcagcatggaactggataagacaatctcccagccgaggtttggaatggctggggcgcacgtactatcgatccaaatggtataatgactacgcagtttccgtaaagtcacggattactataaatccggacacatcaaaaaatcaattcagtctgcagctcaactctgtaacgccagaagataccgctgtctactattgtgcacgggaggtaactggtgaccttgaggacgcattcgatatatggggccaaggaacgatggtcaccgtatctagtggcggtggtggttctgatatacaaatgacgcaatccccctcttccctttctgcctcggtgggagatcgagtaaccattacgtgcagagcttctcagacgatctggagctacctgaattggtatcaacagagacctggtaaagcgccaaatcttcttatatacgctgccagcagcttgcagtcaggagttccgtccagattcagcggtagggggtctgggaccgattttacattgaccatcagtagtcttcaggctgaagattttgcaacatattactgccaacagtcctattctattcctcaaactttcggacagggaaccaaattggaaataaagcttactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaagcgagggaggaaaaagttgttatacatttttaagcagcctttcatgcgccctgttcagaccacgcaggaggaggacggatgctcttgtcgcttccctgaggaggaggagggcggctgcgagttgagggtcaaattttctcgatccgcggatgcgcctgcatataaacaagggcaaaatcaactctacaatgaacttaacttaggacgacgggaagaatatgatgtactcgataagcgccgaggacgcgatccggaaatgggtggcaaaccacggcgtaaaaatccacaagaggggttatataacgagttacaaaaagacaaaatggccgaagcatattccgaaatcggcatgaagggtgagagacgacgaggcaaaggacatgacggtctctatcaaggcctttcaactgccactaaggatacgtatgatgcacttcatatgcaagccttaccaccacgcaagagaagagaattcgcaacaaacttctctctgctgaaacaagccggagatgtcgaagagaatcctggaccgctcgagatgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggcagcggcaagcctggatctggcgagggaagcaccaagggcgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtcatctgcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaactga

SEQ ID NO: 75 - protein, 959 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKRREFATNFSLLKQAGDVEENPGPLEMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 76 - DNA, 2880 bp, Artificial sequence
  Synthetic construct
atgctgctgctggtcactagtctgcttttgtgtgagctgccacaccctgcatttttgcttataccacaggtgcagttgcaacaatctggacctggtctcgtcaaaccttcccagacccttagcttgacgtgtgcgattagtggcgatagcgtcagtagcaacagtgcagcatggaactggataagacaatctcccagccgaggtttggaatggctggggcgcacgtactatcgatccaaatggtataatgactacgcagtttccgtaaagtcacggattactataaatccggacacatcaaaaaatcaattcagtctgcagctcaactctgtaacgccagaagataccgctgtctactattgtgcacgggaggtaactggtgaccttgaggacgcattcgatatatggggccaaggaacgatggtcaccgtatctagtggcggtggtggttctgatatacaaatgacgcaatccccctcttccctttctgcctcggtgggagatcgagtaaccattacgtgcagagcttctcagacgatctggagctacctgaattggtatcaacagagacctggtaaagcgccaaatcttcttatatacgctgccagcagcttgcagtcaggagttccgtccagattcagcggtagggggtctgggaccgattttacattgaccatcagtagtcttcaggctgaagattttgcaacatattactgccaacagtcctattctattcctcaaactttcggacagggaaccaaattggaaataaagcttactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaagcgagggaggaaaaagttgttatacatttttaagcagcctttcatgcgccctgttcagaccacgcaggaggaggacggatgctcttgtcgcttccctgaggaggaggagggcggctgcgagttgagggtcaaattttctcgatccgcggatgcgcctgcatataaacaagggcaaaatcaactctacaatgaacttaacttaggacgacgggaagaatatgatgtactcgataagcgccgaggacgcgatccggaaatgggtggcaaaccacggcgtaaaaatccacaagaggggttatataacgagttacaaaaagacaaaatggccgaagcatattccgaaatcggcatgaagggtgagagacgacgaggcaaaggacatgacggtctctatcaaggcctttcaactgccactaaggatacgtatgatgcacttcatatgcaagccttaccaccacgcaagagaagagaattcgcaacaaacttctctctgctgaaacaagccggagatgtcgaagagaatcctggaccgctcgagatgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggcagcggcaagcctggatctggcgagggaagcaccaagggcgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtcatctgcggccgccgtgattacacacaagtggacaacaagcctgagcgccaagttcaagtgcaccgccggcaacaaggtgtccaaagaaagcagcgtggaacccgtgtcttgccccgagaaaggcctggacatctacctgatcatcggcatctgtggcggcggaagcctgctgatggtttttgtggccctgctggtgttctacatcaccaagcggaagaagcagcggagcagacggaacgacgaggaactggaaacaagagcccacagagtggccaccgaggaaagaggcagaaagccccaccagattccagccagcacacctcagaatcccgccacatctcaacaccctccacctccacctggacacagatctcaggccccatctcatagacctccaccacctggccatagagtgcagcaccagcctcagaaaagacctcctgctcctagcggaacacaggtgcaccagcaaaaaggccctccactgcctagacctagggtgcagcctaaacctcctcatggcgccgctgagaatagcctgtctcctagcagcaactga

SEQ ID NO: 77 - protein, 846 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKRREFATNFSLLKQAGDVEENPGPLEMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVKR

SEQ ID NO: 78 - DNA, 2541 bp, Artificial sequence
  Synthetic construct
atgctgctgctggtcactagtctgcttttgtgtgagctgccacaccctgcatttttgcttataccacaggtgcagttgcaacaatctggacctggtctcgtcaaaccttcccagacccttagcttgacgtgtgcgattagtggcgatagcgtcagtagcaacagtgcagcatggaactggataagacaatctcccagccgaggtttggaatggctggggcgcacgtactatcgatccaaatggtataatgactacgcagtttccgtaaagtcacggattactataaatccggacacatcaaaaaatcaattcagtctgcagctcaactctgtaacgccagaagataccgctgtctactattgtgcacgggaggtaactggtgaccttgaggacgcattcgatatatggggccaaggaacgatggtcaccgtatctagtggcggtggtggttctgatatacaaatgacgcaatccccctcttccctttctgcctcggtgggagatcgagtaaccattacgtgcagagcttctcagacgatctggagctacctgaattggtatcaacagagacctggtaaagcgccaaatcttcttatatacgctgccagcagcttgcagtcaggagttccgtccagattcagcggtagggggtctgggaccgattttacattgaccatcagtagtcttcaggctgaagattttgcaacatattactgccaacagtcctattctattcctcaaactttcggacagggaaccaaattggaaataaagcttactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaagcgagggaggaaaaagttgttatacatttttaagcagcctttcatgcgccctgttcagaccacgcaggaggaggacggatgctcttgtcgcttccctgaggaggaggagggcggctgcgagttgagggtcaaattttctcgatccgcggatgcgcctgcatataaacaagggcaaaatcaactctacaatgaacttaacttaggacgacgggaagaatatgatgtactcgataagcgccgaggacgcgatccggaaatgggtggcaaaccacggcgtaaaaatccacaagaggggttatataacgagttacaaaaagacaaaatggccgaagcatattccgaaatcggcatgaagggtgagagacgacgaggcaaaggacatgacggtctctatcaaggcctttcaactgccactaaggatacgtatgatgcacttcatatgcaagccttaccaccacgcaagagaagagaattcgcaacaaacttctctctgctgaaacaagccggagatgtcgaagagaatcctggaccgctcgagatgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggcagcggcaagcctggatctggcgagggaagcaccaagggcgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtcatctgcggccgccattgaggtcatgtacccaccaccttacctcgacaacgaaaaatcaaacgggacgattattcacgtcaaaggcaagcatctgtgcccgtcacctctgttccccggaccaagcaaaccgttctgggtgcttgttgtcgtcggcggggtccttgcttgttactcacttctggttaccgttgcttttatcattttttgggtgaagcggtaa

SEQ ID NO: 79 - protein, 515 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKRREFATNFSLLKQAGDVEENPGPLEPPPCCCS

SEQ ID NO: 80 - DNA, 1548 bp, Artificial sequence
  Synthetic construct
atgctgctgctggtcactagtctgcttttgtgtgagctgccacaccctgcatttttgcttataccacaggtgcagttgcaacaatctggacctggtctcgtcaaaccttcccagacccttagcttgacgtgtgcgattagtggcgatagcgtcagtagcaacagtgcagcatggaactggataagacaatctcccagccgaggtttggaatggctggggcgcacgtactatcgatccaaatggtataatgactacgcagtttccgtaaagtcacggattactataaatccggacacatcaaaaaatcaattcagtctgcagctcaactctgtaacgccagaagataccgctgtctactattgtgcacgggaggtaactggtgaccttgaggacgcattcgatatatggggccaaggaacgatggtcaccgtatctagtggcggtggtggttctgatatacaaatgacgcaatccccctcttccctttctgcctcggtgggagatcgagtaaccattacgtgcagagcttctcagacgatctggagctacctgaattggtatcaacagagacctggtaaagcgccaaatcttcttatatacgctgccagcagcttgcagtcaggagttccgtccagattcagcggtagggggtctgggaccgattttacattgaccatcagtagtcttcaggctgaagattttgcaacatattactgccaacagtcctattctattcctcaaactttcggacagggaaccaaattggaaataaagcttactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaagcgagggaggaaaaagttgttatacatttttaagcagcctttcatgcgccctgttcagaccacgcaggaggaggacggatgctcttgtcgcttccctgaggaggaggagggcggctgcgagttgagggtcaaattttctcgatccgcggatgcgcctgcatataaacaagggcaaaatcaactctacaatgaacttaacttaggacgacgggaagaatatgatgtactcgataagcgccgaggacgcgatccggaaatgggtggcaaaccacggcgtaaaaatccacaagaggggttatataacgagttacaaaaagacaaaatggccgaagcatattccgaaatcggcatgaagggtgagagacgacgaggcaaaggacatgacggtctctatcaaggcctttcaactgccactaaggatacgtatgatgcacttcatatgcaagccttaccaccacgcaagagaagagaattcgcaacaaacttctctctgctgaaacaagccggagatgtcgaagagaatcctggaccgctcgagccgccaccatgctgctgctcgtga

SEQ ID NO: 81 - protein, 961 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKRREFATNFSLLKQAGDVEENPGPLEMVLLVTSLLLCELPHPAFLLIPDTEVQLVESGGGLVKPGGSLKLSCAASGFAFSIYDMSWVRQTPEKRLEWVAYISSGGGTYYPDTVKGRFTISRDNAKNTLYLQMSSLKSEDTAMYYCARHSGYGTHWGVLFAYWGQGTLVTVSAGGGGSGGGGSGGGGSDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKAAAIEVMYPPPYLDNEKSNGTIIHVKGKHLCPSPLFPGPSKPFWVLVVVGGVLACYSLLVTVAFIIFWVKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 82 - DNA, 2886 bp, Artificial sequence
  Synthetic construct
atgctgctgctggtcactagtctgcttttgtgtgagctgccacaccctgcatttttgcttataccacaggtgcagttgcaacaatctggacctggtctcgtcaaaccttcccagacccttagcttgacgtgtgcgattagtggcgatagcgtcagtagcaacagtgcagcatggaactggataagacaatctcccagccgaggtttggaatggctggggcgcacgtactatcgatccaaatggtataatgactacgcagtttccgtaaagtcacggattactataaatccggacacatcaaaaaatcaattcagtctgcagctcaactctgtaacgccagaagataccgctgtctactattgtgcacgggaggtaactggtgaccttgaggacgcattcgatatatggggccaaggaacgatggtcaccgtatctagtggcggtggtggttctgatatacaaatgacgcaatccccctcttccctttctgcctcggtgggagatcgagtaaccattacgtgcagagcttctcagacgatctggagctacctgaattggtatcaacagagacctggtaaagcgccaaatcttcttatatacgctgccagcagcttgcagtcaggagttccgtccagattcagcggtagggggtctgggaccgattttacattgaccatcagtagtcttcaggctgaagattttgcaacatattactgccaacagtcctattctattcctcaaactttcggacagggaaccaaattggaaataaagcttactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaagcgagggaggaaaaagttgttatacatttttaagcagcctttcatgcgccctgttcagaccacgcaggaggaggacggatgctcttgtcgcttccctgaggaggaggagggcggctgcgagttgagggtcaaattttctcgatccgcggatgcgcctgcatataaacaagggcaaaatcaactctacaatgaacttaacttaggacgacgggaagaatatgatgtactcgataagcgccgaggacgcgatccggaaatgggtggcaaaccacggcgtaaaaatccacaagaggggttatataacgagttacaaaaagacaaaatggccgaagcatattccgaaatcggcatgaagggtgagagacgacgaggcaaaggacatgacggtctctatcaaggcctttcaactgccactaaggatacgtatgatgcacttcatatgcaagccttaccaccacgcaagagaagagaattcgcaacaaacttctctctgctgaaacaagccggagatgtcgaagagaatcctggaccgctcgagatggttttattggtcacatcactgctcctctgtgaactgcctcatcccgcttttttattgattcccgacactgaagtccagctcgtggaatctggagggggcctggtgaaacctgggggatctctcaaactgtcttgtgccgcttctggctttgcttttagcatctacgacatgtcctgggtccggcagacacctgaaaaacgcctggagtgggtcgcctacatttctagtgggggcggaacatactaccccgataccgtgaagggacgctttacaatttctagggataacgccaaaaacaccctgtacctccagatgtcatccctgaaatctgaggatactgccatgtactactgtgctaggcattctggctacggaacacattggggagtgctcttcgcttactggggccaggggactctcgtcactgtctctgctggcgggggaggctctggcggaggcggttccggaggcggagggagtgatattcagatgactcagaccacctcttctctgtccgcttctctgggcgatagagtgacaatctcctgtcgggcatcacaggatattagcaattacctgaactggtaccagcagaaacccgatggaaccgtcaaactgctcatctactacacctccatcctccactctggcgtgccatctcgattttctggatctggctctggaaccgactactctctcacaatctccaacctggaacaggaggattttgccacctacttttgtcagcagggcaatactctgccttggacctttgggggcggaaccaaactggaaatcaaggcggccgccattgaggtcatgtacccaccaccttacctcgacaacgaaaaatcaaacgggacgattattcacgtcaaaggcaagcatctgtgcccgtcacctctgttccccggaccaagcaaaccgttctgggtgcttgttgtcgtcggcggggtccttgcttgttactcacttctggttaccgttgcttttatcattttttgggtgaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaactaa

SEQ ID NO: 83 - protein, 964 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKRREFATNFSLLKQAGDVEENPGPLEMVLLVTSLLLCELPHPAFLLIPDTEVQLVESGGGLVKPGGSLKLSCAASGFAFSIYDMSWVRQTPEKRLEWVAYISSGGGTYYPDTVKGRFTISRDNAKNTLYLQMSSLKSEDTAMYYCARHSGYGTHWGVLFAYWGQGTLVTVSAGGGGSGGGGSGGGGSDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKAAATTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 84 - DNA, 2895 bp, Artificial sequence
  Synthetic construct
atgctgctgctggtcactagtctgcttttgtgtgagctgccacaccctgcatttttgcttataccacaggtgcagttgcaacaatctggacctggtctcgtcaaaccttcccagacccttagcttgacgtgtgcgattagtggcgatagcgtcagtagcaacagtgcagcatggaactggataagacaatctcccagccgaggtttggaatggctggggcgcacgtactatcgatccaaatggtataatgactacgcagtttccgtaaagtcacggattactataaatccggacacatcaaaaaatcaattcagtctgcagctcaactctgtaacgccagaagataccgctgtctactattgtgcacgggaggtaactggtgaccttgaggacgcattcgatatatggggccaaggaacgatggtcaccgtatctagtggcggtggtggttctgatatacaaatgacgcaatccccctcttccctttctgcctcggtgggagatcgagtaaccattacgtgcagagcttctcagacgatctggagctacctgaattggtatcaacagagacctggtaaagcgccaaatcttcttatatacgctgccagcagcttgcagtcaggagttccgtccagattcagcggtagggggtctgggaccgattttacattgaccatcagtagtcttcaggctgaagattttgcaacatattactgccaacagtcctattctattcctcaaactttcggacagggaaccaaattggaaataaagcttactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaagcgagggaggaaaaagttgttatacatttttaagcagcctttcatgcgccctgttcagaccacgcaggaggaggacggatgctcttgtcgcttccctgaggaggaggagggcggctgcgagttgagggtcaaattttctcgatccgcggatgcgcctgcatataaacaagggcaaaatcaactctacaatgaacttaacttaggacgacgggaagaatatgatgtactcgataagcgccgaggacgcgatccggaaatgggtggcaaaccacggcgtaaaaatccacaagaggggttatataacgagttacaaaaagacaaaatggccgaagcatattccgaaatcggcatgaagggtgagagacgacgaggcaaaggacatgacggtctctatcaaggcctttcaactgccactaaggatacgtatgatgcacttcatatgcaagccttaccaccacgcaagagaagagaattcgcaacaaacttctctctgctgaaacaagccggagatgtcgaagagaatcctggaccgctcgagatggttttattggtcacatcactgctcctctgtgaactgcctcatcccgcttttttattgattcccgacactgaagtccagctcgtggaatctggagggggcctggtgaaacctgggggatctctcaaactgtcttgtgccgcttctggctttgcttttagcatctacgacatgtcctgggtccggcagacacctgaaaaacgcctggagtgggtcgcctacatttctagtgggggcggaacatactaccccgataccgtgaagggacgctttacaatttctagggataacgccaaaaacaccctgtacctccagatgtcatccctgaaatctgaggatactgccatgtactactgtgctaggcattctggctacggaacacattggggagtgctcttcgcttactggggccaggggactctcgtcactgtctctgctggcgggggaggctctggcggaggcggttccggaggcggagggagtgatattcagatgactcagaccacctcttctctgtccgcttctctgggcgatagagtgacaatctcctgtcgggcatcacaggatattagcaattacctgaactggtaccagcagaaacccgatggaaccgtcaaactgctcatctactacacctccatcctccactctggcgtgccatctcgattttctggatctggctctggaaccgactactctctcacaatctccaacctggaacaggaggattttgccacctacttttgtcagcagggcaatactctgccttggacctttgggggcggaaccaaactggaaatcaaggcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaactga

SEQ ID NO: 85 - protein, 960 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKRREFATNFSLLKQAGDVEENPGPLEMVLLVTSLLLCELPHPAFLLIPDTEVQLVESGGGLVKPGGSLKLSCAASGFAFSIYDMSWVRQTPEKRLEWVAYISSGGGTYYPDTVKGRFTISRDNAKNTLYLQMSSLKSEDTAMYYCARHSGYGTHWGVLFAYWGQGTLVTVSAGGGGSGGGGSGGGGSDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKAAAVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 86 - DNA, 2883 bp, Artificial sequence
  Synthetic construct
atgctgctgctggtcactagtctgcttttgtgtgagctgccacaccctgcatttttgcttataccacaggtgcagttgcaacaatctggacctggtctcgtcaaaccttcccagacccttagcttgacgtgtgcgattagtggcgatagcgtcagtagcaacagtgcagcatggaactggataagacaatctcccagccgaggtttggaatggctggggcgcacgtactatcgatccaaatggtataatgactacgcagtttccgtaaagtcacggattactataaatccggacacatcaaaaaatcaattcagtctgcagctcaactctgtaacgccagaagataccgctgtctactattgtgcacgggaggtaactggtgaccttgaggacgcattcgatatatggggccaaggaacgatggtcaccgtatctagtggcggtggtggttctgatatacaaatgacgcaatccccctcttccctttctgcctcggtgggagatcgagtaaccattacgtgcagagcttctcagacgatctggagctacctgaattggtatcaacagagacctggtaaagcgccaaatcttcttatatacgctgccagcagcttgcagtcaggagttccgtccagattcagcggtagggggtctgggaccgattttacattgaccatcagtagtcttcaggctgaagattttgcaacatattactgccaacagtcctattctattcctcaaactttcggacagggaaccaaattggaaataaagcttactactacaccggcacccaggccacctacacctgcaccgaccattgcttcacaaccactcagtctccgacctgaagcctgtagacctgctgctggcggtgcagtccataccagaggactcgattttgcttgcgacatttatatatgggcacctttggcaggaacctgcggggtattgcttctttccctggtgatcacactgtattgtaagcgagggaggaaaaagttgttatacatttttaagcagcctttcatgcgccctgttcagaccacgcaggaggaggacggatgctcttgtcgcttccctgaggaggaggagggcggctgcgagttgagggtcaaattttctcgatccgcggatgcgcctgcatataaacaagggcaaaatcaactctacaatgaacttaacttaggacgacgggaagaatatgatgtactcgataagcgccgaggacgcgatccggaaatgggtggcaaaccacggcgtaaaaatccacaagaggggttatataacgagttacaaaaagacaaaatggccgaagcatattccgaaatcggcatgaagggtgagagacgacgaggcaaaggacatgacggtctctatcaaggcctttcaactgccactaaggatacgtatgatgcacttcatatgcaagccttaccaccacgcaagagaagagaattcgcaacaaacttctctctgctgaaacaagccggagatgtcgaagagaatcctggaccgctcgagatggttttattggtcacatcactgctcctctgtgaactgcctcatcccgcttttttattgattcccgacactgaagtccagctcgtggaatctggagggggcctggtgaaacctgggggatctctcaaactgtcttgtgccgcttctggctttgcttttagcatctacgacatgtcctgggtccggcagacacctgaaaaacgcctggagtgggtcgcctacatttctagtgggggcggaacatactaccccgataccgtgaagggacgctttacaatttctagggataacgccaaaaacaccctgtacctccagatgtcatccctgaaatctgaggatactgccatgtactactgtgctaggcattctggctacggaacacattggggagtgctcttcgcttactggggccaggggactctcgtcactgtctctgctggcgggggaggctctggcggaggcggttccggaggcggagggagtgatattcagatgactcagaccacctcttctctgtccgcttctctgggcgatagagtgacaatctcctgtcgggcatcacaggatattagcaattacctgaactggtaccagcagaaacccgatggaaccgtcaaactgctcatctactacacctccatcctccactctggcgtgccatctcgattttctggatctggctctggaaccgactactctctcacaatctccaacctggaacaggaggattttgccacctacttttgtcagcagggcaatactctgccttggacctttgggggcggaaccaaactggaaatcaaggcggccgccgtgattacacacaagtggacaacaagcctgagcgccaagttcaagtgcaccgccggcaacaaggtgtccaaagaaagcagcgtggaacccgtgtcttgccccgagaaaggcctggacatctacctgatcatcggcatctgtggcggcggaagcctgctgatggtttttgtggccctgctggtgttctacatcaccaagcggaagaagcagcggagcagacggaacgacgaggaactggaaacaagagcccacagagtggccaccgaggaaagaggcagaaagccccaccagattccagccagcacacctcagaatcccgccacatctcaacaccctccacctccacctggacacagatctcaggccccatctcatagacctccaccacctggccatagagtgcagcaccagcctcagaaaagacctcctgctcctagcggaacacaggtgcaccagcaaaaaggccctccactgcctagacctagggtgcagcctaaacctcctcatggcgccgctgagaatagcctgtctcctagcagcaactga

SEQ ID NO: 87 - DNA, 45 bp, Artificial sequence
  Synthetic construct
  misc_feature (4)..(45): These nucleotides may be absent
ggsggsggsggsggsggsggsggsggsggsggsggsggsggsggs

SEQ ID NO: 88 - DNA, 45 bp, Artificial sequence
  Synthetic construct
  misc_feature (4)..(45): These nucleotides may be absent
sggsggsggsggsggsggsggsggsggsggsggsggsggsggsgg

SEQ ID NO: 89 - DNA, 60 bp, Artificial sequence
  Synthetic construct
  misc_feature (5)..(60): These nucleotides may be absent
gggsgggsgggsgggsgggsgggsgggsgggsgggsgggsgggsgggsgggsgggsgggs

SEQ ID NO: 90 - DNA, 60 bp, Artificial sequence
  Synthetic construct
  misc_feature (5)..(60): These nucleotides may be absent
sgggsgggsgggsgggsgggsgggsgggsgggsgggsgggsgggsgggsgggsgggsggg

SEQ ID NO: 91 - DNA, 75 bp, Artificial sequence
  Synthetic construct
  misc_feature (6)..(75): These nucleotides may be absent
ggggsggggsggggsggggsggggsggggsggggsggggsggggsggggsggggsggggsggggsggggsggggs

SEQ ID NO: 92 - DNA, 1704 bp, Artificial sequence; identical to SEQ ID NO: 41
  Synthetic construct

SEQ ID NO: 93 - DNA, 1830 bp, Artificial sequence; identical to SEQ ID NO: 42
  Synthetic construct

SEQ ID NO: 94 - DNA, 1830 bp, Artificial sequence; identical to SEQ ID NO: 43
  Synthetic construct

SEQ ID NO: 95 - DNA, 1830 bp, Artificial sequence; identical to SEQ ID NO: 44
  Synthetic construct

SEQ ID NO: 96 - DNA, 1695 bp, Artificial sequence
  Synthetic construct
atgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggcagcggcaagcctggatctggcgagggaagcaccaagggcgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtcatctgcggccgccattgaggtcatgtacccaccaccttacctcgacaacgaaaaatcaaacgggacgattattcacgtcaaaggcaagcatctgtgcccgtcacctctgttccccggaccaagcaaaccgttctgggtgcttgttgtcgtcggcggggtccttgcttgttactcacttctggttaccgttgcttttatcattttttgggtgaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaacagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataa

SEQ ID NO: 97 - DNA, 1677 bp, Artificial sequence; identical to SEQ ID NO: 45
  Synthetic construct

SEQ ID NO: 98 - DNA, 1803 bp, Artificial sequence; identical to SEQ ID NO: 46
  Synthetic construct

SEQ ID NO: 99 - DNA, 1803 bp, Artificial sequence; identical to SEQ ID NO: 47
  Synthetic construct

SEQ ID NO: 100 - DNA, 1803 bp, Artificial sequence; identical to SEQ ID NO: 48
  Synthetic construct

SEQ ID NO: 101 - DNA, 1356 bp, Artificial sequence; identical to SEQ ID NO: 49
  Synthetic construct

SEQ ID NO: 102 - DNA, 1365 bp, Artificial sequence; identical to SEQ ID NO: 50
  Synthetic construct

SEQ ID NO: 103 - DNA, 1353 bp, Artificial sequence; identical to SEQ ID NO: 51
  Synthetic construct

SEQ ID NO: 104 - DNA, 1329 bp, Artificial sequence; identical to SEQ ID NO: 52
  Synthetic construct

SEQ ID NO: 105 - DNA, 1338 bp, Artificial sequence; identical to SEQ ID NO: 53
  Synthetic construct

SEQ ID NO: 106 - DNA, 1359 bp, Artificial sequence; identical to SEQ ID NO: 54
  Synthetic construct

SEQ ID NO: 107 - DNA, 1368 bp, Artificial sequence; identical to SEQ ID NO: 55
  Synthetic construct

SEQ ID NO: 108 - DNA, 1356 bp, Artificial sequence; identical to SEQ ID NO: 56
  Synthetic construct

SEQ ID NO: 109 - DNA, 2064 bp, Artificial sequence; identical to SEQ ID NO: 57
  Synthetic construct

SEQ ID NO: 110 - DNA, 2073 bp, Artificial sequence; identical to SEQ ID NO: 58
  Synthetic construct

SEQ ID NO: 111 - DNA, 2403 bp, Artificial sequence
  Synthetic construct
atgctgctgctggtcacatctctgctgctgtgcgaactgccccatcctgcctttctgctgatccccgatatcgtgctgacacagagccctgccattctgtctgctagccctggcgagaaagtgaccatgacctgtagagccagcagctccgtgaactacatggactggtatcagaagaagcccggcagcagccccaagccttggatctacgctacaagcaatctggccagcggcgtgccagccagattttctggttctggcagcggcaccagctacagcctgacaatctctagagtggaagccgaggacgccgccacctactattgccagcagtggtccttcaatcctcctacctttggcggaggcaccaagctggaaatcaagggctctacatctggcggcggttctggtggtggaagcggaggcggaggatcttctgaagttcagctgcagcagtctggcgccgaacttgtgaaacctggcgcctctgtgaagatgagctgcaaggcctctggctacaccttcacaagctacaacatgcactgggtcaagcagacccctggacagggactcgaatggatcggagccatctatcccggcaacggcgacacctcctacaaccagaagttcaagggcaaagccacactgaccgccgacaagagcagctctacagcctacatgcagctgagcagcctgaccagcgaggacagcgccgattactactgcgccagaagcaactactacggcagctcctactggttcttcgatgtgtggggagccggcaccacagtgacagtgtctagcgagcctaagagctgcgacaagacccacacctgtcctccatgtcctgctcctccagtggccggaccttccgtgtttctgttccctccaaagcctaaggacaccctgatgatcagcaggacccctgaagtgacctgtgtggtggtcgatgtgtcccacgaggacccagaagtgaagttcaattggtacgtggacggcgtggaagtgcacaacgccaagaccaagcctagagaggaacagtaccagagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttctcctgttctgtgatgcacgaggccctgcacaaccactacacccagaaaagcctgtctctgagccccggcaagaaggaccctaaagcggccgccattgaggtcatgtacccaccaccttacctcgacaacgaaaaatcaaacgggacgattattcacgtcaaaggcaagcatctgtgcccgtcacctctgttccccggaccaagcaaaccgttctgggtgcttgttgtcgtcggcggggtccttgcttgttactcacttctggttaccgttgcttttatcattttttgggtgaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaacagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataa

SEQ ID NO: 112 - DNA, 2412 bp, Artificial sequence
  Synthetic construct
atgctgctgctggtcacatctctgctgctgtgcgaactgccccatcctgcctttctgctgatccccgatatcgtgctgacacagagccctgccattctgtctgctagccctggcgagaaagtgaccatgacctgtagagccagcagctccgtgaactacatggactggtatcagaagaagcccggcagcagccccaagccttggatctacgctacaagcaatctggccagcggcgtgccagccagattttctggttctggcagcggcaccagctacagcctgacaatctctagagtggaagccgaggacgccgccacctactattgccagcagtggtccttcaatcctcctacctttggcggaggcaccaagctggaaatcaagggctctacatctggcggcggttctggtggtggaagcggaggcggaggatcttctgaagttcagctgcagcagtctggcgccgaacttgtgaaacctggcgcctctgtgaagatgagctgcaaggcctctggctacaccttcacaagctacaacatgcactgggtcaagcagacccctggacagggactcgaatggatcggagccatctatcccggcaacggcgacacctcctacaaccagaagttcaagggcaaagccacactgaccgccgacaagagcagctctacagcctacatgcagctgagcagcctgaccagcgaggacagcgccgattactactgcgccagaagcaactactacggcagctcctactggttcttcgatgtgtggggagccggcaccacagtgacagtgtctagcgagcctaagagctgcgacaagacccacacctgtcctccatgtcctgctcctccagtggccggaccttccgtgtttctgttccctccaaagcctaaggacaccctgatgatcagcaggacccctgaagtgacctgtgtggtggtcgatgtgtcccacgaggacccagaagtgaagttcaattggtacgtggacggcgtggaagtgcacaacgccaagaccaagcctagagaggaacagtaccagagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttctcctgttctgtgatgcacgaggccctgcacaaccactacacccagaaaagcctgtctctgagccccggcaagaaggaccctaaagcggccgcaacaacaacaccagctcctagacctccaactcctgctcctacaatcgccagccagcctctgtctctcagacctgaagcctgtagacctgctgctggcggagctgtgcataccagaggactggatttcgcctgcgacatctacatttgggcccctctggctggaacatgtggcgtgctgctgctgtctctggtcatcaccctgtactgcaagcggaagaagcagcggagcagacggaacgacgaggaactggaaacaagagcccacagagtggccaccgaggaaagaggcagaaagccccaccagattccagcctccacacctcagaatcccgccacatctcaacaccctccacctccacctggacacagatctcaggccccatctcatagaccaccacctccaggacacagagtgcagcaccagcctcagaaaagacctcctgcacctagcggaacacaggtgcaccagcaaaaaggccctccactgcctagacctagggtgcagcctaaacctcctcatggcgccgctgagaatagcctgtctcctagcagcaacagagtgaagttctctagatctgccgacgctcccgcctacaagcagggccagaatcagctgtacaacgagctgaacctggggagaagagaagagtacgacgtgctggataagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggccacgatggactgtatcagggcctgagcacagccaccaaggatacctatgatgccctgcacatgcaggccctgcctccaagataa

SEQ ID NO: 113 - DNA, 1017 bp, Artificial sequence
  Synthetic construct
atgctgctgctcgtgacatctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggcagcggcaagcctggatctggcgagggaagcaccaagggcgaagtgaaactgcaggaaagcggccctggactggtggccccaagccagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgctatggactactggggccagggcaccagcgtgaccgtgtcatctgcggccgccattgaggtcatgtacccaccaccttacctcgacaacgaaaaatcaaacgggacgattattcacgtcaaaggcaagcatctgtgcccgtcacctctgttccccggaccaagcaaaccgttctgggtgcttgttgtcgtcggcggggtccttgcttgttactcacttctggttaccgttgcttttatcattttttgggtgaagcggtaa

SEQ ID NO: 114 - protein, 567 aa, Artificial sequence; identical to SEQ ID NO: 12
  Synthetic construct

SEQ ID NO: 115 - protein, 609 aa, Artificial sequence; identical to SEQ ID NO: 13
  Synthetic construct

SEQ ID NO: 116 - protein, 609 aa, Artificial sequence; identical to SEQ ID NO: 14
  Synthetic construct

SEQ ID NO: 117 - protein, 609 aa, Artificial sequence; identical to SEQ ID NO: 15
  Synthetic construct

SEQ ID NO: 118 - protein, 564 aa, Artificial sequence
  Synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 558 aa, Artificial sequence; identical to SEQ ID NO: 16
  Synthetic construct

SEQ ID NO: 120 - protein, 488 aa, Artificial sequence
  Synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNKRGRKKLLYIFKQPFMRPVQTTQEEDGCSCRFPEEEEGGCEL

SEQ ID NO: 121 - protein, 600 aa, Artificial sequence; identical to SEQ ID NO: 18
  Synthetic construct

SEQ ID NO: 122 - protein, 600 aa, Artificial sequence; identical to SEQ ID NO: 19
  Synthetic construct

SEQ ID NO: 123 - protein, 452 aa, Artificial sequence; identical to SEQ ID NO: 20
  Synthetic construct

SEQ ID NO: 124 - protein, 455 aa, Artificial sequence; identical to SEQ ID NO: 21
  Synthetic construct

SEQ ID NO: 125 - protein, 451 aa, Artificial sequence; identical to SEQ ID NO: 22
  Synthetic construct

SEQ ID NO: 126 - protein, 443 aa, Artificial sequence; identical to SEQ ID NO: 23
  Synthetic construct

SEQ ID NO: 127 - protein, 446 aa, Artificial sequence; identical to SEQ ID NO: 24
  Synthetic construct

SEQ ID NO: 128 - protein, 453 aa, Artificial sequence; identical to SEQ ID NO: 25
  Synthetic construct

SEQ ID NO: 129 - protein, 456 aa, Artificial sequence; identical to SEQ ID NO: 26
  Synthetic construct

SEQ ID NO: 130 - protein, 452 aa, Artificial sequence; identical to SEQ ID NO: 27
  Synthetic construct

SEQ ID NO: 131 - protein, 688 aa, Artificial sequence; identical to SEQ ID NO: 28
  Synthetic construct

SEQ ID NO: 132 - protein, 691 aa, Artificial sequence; identical to SEQ ID NO: 29
  Synthetic construct

SEQ ID NO: 133 - protein, 800 aa, Artificial sequence
  Synthetic construct
MLLLVTSLLLCELPHPAFLLIPDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKAAAIEVMYPPPYLDNEKSNGTIIHVKGKHLCPSPLFPGPSKPFWVLVVVGGVLACYSLLVTVAFIIFWVKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 803 aa, Artificial sequence
  Synthetic construct
MLLLVTSLLLCELPHPAFLLIPDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKAAATTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 338 aa, Artificial sequence
  Synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVKR

(19) United States
(12) Patent Application Publication
(10) Pub. No.: US 2023/0183312 A1
(43) Pub. Date: Jun. 15, 2023

(54) CHIMERIC ANTIGEN RECEPTORS WITH CD2 ACTIVATION

(71) Applicant: The Board of Trustees of the Leland Stanford Junior University, Stanford, CA (US)

(72) Inventors: Robbie G. MAJZNER, Palo Alto, CA (US); Crystal L. MACKALL, Stanford, CA (US); Aidan TOUSLEY, Stanford, CA (US); Michelle MONJE-DEISSEROTH, Stanford, CA (US); Louai LABANIEH, Stanford, CA (US); Christopher MOUNT, Stanford, CA (US)

(21) Appl. No.: 17/904,219

(22) PCT Filed: Feb. 12, 2021
(86) PCT No.: PCT/US2021/018027

§ 371 Date: Aug. 12, 2022

Related U.S. Application Data
(60) Provisional application No. 62/976,997, filed on Feb. 14, 2020.
(60) Provisional application No. 63/109,831, filed on Nov. 4, 2020.

(51) Int. Cl. C07K 14/705; C07K 16/28; A61P 35/00

(52) U.S. Cl. CPC C07K 14/70507; C07K 16/2896; A61P 35/00; C07K 2319/02; C07K 2319/03; C07K 2317/622

(57) ABSTRACT

Disclosed are chimeric antigen receptors comprising a CD2 co-stimulatory domain that retain function against CD58⁻ and CD58^{low} tumor cells, and CD2 co-stimulatory receptors that promote CAR function against CD58⁻ and CD58^{low} tumor cells.

### CROSS-REFERENCE TO RELATED APPLICATIONS

This application claims priority to U.S. Provisional Patent Application Nos. 62/976,997, filed Feb. 14, 2020, and 63/109,831, filed Nov. 4, 2020, the disclosures of which are incorporated by reference herein in their entireties, including any drawings.

### STATEMENT OF RIGHTS TO INVENTIONS MADE UNDER FEDERALLY SPONSORED RESEARCH AND DEVELOPMENT

This invention was made with Government support under contract CA049605 awarded by the National Institutes of Health. The Government has certain rights in the invention.

### INCORPORATION OF THE SEQUENCE LISTING

This application contains a Sequence Listing, which is hereby incorporated herein by reference in its entirety. The accompanying Sequence Listing text file, named “078430_514001WO_Sequence_Listing_ST25” was created on Feb. 12, 2021 and is 423 KB.

### BACKGROUND OF THE DISCLOSURE

For patients with B-cell lymphoma, including those who have relapsed after receiving traditional chemotherapy regimens, immunotherapeutic approaches have shown tremendous clinical efficacy. In a recent Phase II study of 111 patients with refractory B-cell lymphoma, of whom 101 were administered CAR-T cell (chimeric antigen receptor T cell) therapy targeting CD19, 40% of patients showed complete remission of disease 15 months after treatment (S. S. Neelapu et al., N Engl J Med (2017) 377:2531-44). Similar results were observed in a separate study, with complete remission observed in 43% and 71% of patients with diffuse large B-cell and follicular lymphoma, respectively (S. J. Schuster et al., N Engl J Med (2017) 377:2545-54). While approximately half of patients receiving axicabtagene ciloleucel (Axi-cel) achieve complete responses, a significant subset of patients experience disease progression (F. L. Locke et al., Lancet Oncol (2019) 20(1):31-42). T cells are stimulated by binding between the T cell receptor (CD3) and an MHC (major histocompatability antigen) protein presenting a non-self antigen. The T cell response is greatly improved when co-stimulation occurs, due to co-stimulatory factors such as 4-1BB and CD28. However, Axi-cel and Tisagenlecleucel each include a co-stimulatory domain built into the CAR (CD28 and 4-1BB, respectively), yet relapse and disease progression continue to occur.

An urgent medical need exists to identify the cause of disease progression, and to treat patients who fail to obtain remission using existing CAR-T therapy.

All publications and patent applications mentioned in this disclosure are herein incorporated by reference to the same extent as if each individual publication or patent application was specifically and individually indicated to be incorporated by reference. No admission is made that any reference cited herein constitutes prior art. The discussion of the references states what their authors assert, and the inventors reserve the right to challenge the accuracy and pertinence of the cited documents. It will be understood that, although a number of information sources (including scientific journal articles, patent documents, and textbooks) are referred to herein, this reference does not constitute an admission that any of these documents forms part of the common general knowledge in the art.

### SUMMARY OF THE DISCLOSURE

A substantial fraction of the patients who relapse or fail to respond to CAR-T treatment show reduced or altered expression of functional CD58 in their tumor tissue. In these patients, tumor cell CD58 expression is reduced or absent, or CD58 is expressed in a mutated form with reduced or absent ability to bind CD2. Unexpectedly, existing CAR-T agents rely on tumor cell expression of CD58 for part of their co-stimulation. Provided herein are protective methods and reagents that overcome CAR-T reliance on tumor-expressed CD58, and that restore activity and efficacy to CAR-T therapy. The engineered CAR-T cells of the disclosure can be used to treat cancerous cells in a subject, regardless of whether the cells to be targeted express CD58 at normal levels or reduced levels, or express a mutated or inactive form of CD58, or do not express any form of CD58. The engineered cells of the disclosure in many cases exhibit greater activity than other CAR-T cells lacking a CD2 co-stimulating domain in the CAR construct.

An aspect of the disclosure is a chimeric antigen receptor (CAR), comprising in order from N-terminal to C-terminal: a first antigen binding domain; a spacer domain; a transmembrane domain; and a cytoplasmic signaling domain, wherein the cytoplasmic signaling domain comprises a CD2 signaling domain, a co-stimulating signaling domain, and a CD3ζ activating domain, wherein the co-stimulating signaling domain is not a CD28 signaling domain. In some embodiments, the co-stimulating signaling domain comprises a 4-1BB signaling domain, a CD27 signaling domain, or an OX40 signaling domain. In some embodiments, the co-stimulating signaling domain comprises a 4-1BB signaling domain. In some embodiments, the CAR further comprises a second antigen binding domain. In some embodiments, the first antigen binding domain and the second antigen binding domain are specific for different antigens. In some embodiments, the first antigen binding domain and the second antigen binding domain are specific for different epitopes of the same antigen.

In some embodiments, the spacer domain is selected from the group consisting of a CD8α hinge domain, a CD28 hinge domain, a CTLA-4 hinge domain, IgG1 hinge domain, and an IgG4 hinge domain. In some embodiments, the spacer domain comprises a CD28 hinge domain. In some embodiments, the spacer domain is a synthetic polypeptide spacer having from about 10 to about 50 amino acids. In some embodiments, the synthetic polypeptide spacer is a (GGS)ₙ, (SGG)ₙ, (GGGS)ₙ, (SGGG)ₙ, or (GGGGS)ₙ, where n is about 1 to about 15 (SEQ ID NOs 87-91).

In some embodiments, the transmembrane domain is selected from the group consisting of all or part of the transmembrane domain of the CD3 zeta chain (CD3ζ), CD2, CD8α, CD28, CD40, CTLA4, OX40, PD-1, 4-1BB (CD137), FcERIγ, ICOS (CD278), ILRB (CD122), CTLA-4, and PD-1, and IL-2RG (CD132). In some embodiments, the transmembrane domain comprises a CD8α transmembrane domain. In some embodiments, the transmembrane domain comprises a CD28 transmembrane domain. In some embodiments, the transmembrane domain comprises a CD3ζ transmembrane domain. In some embodiments, the CAR has an amino acid sequence that is at least 70%, 80%, 90%, 95%, 97%, 98%, 99%, or about 100% identical to the sequence of one of SEQ ID NOs: 12 to 19.

In some embodiments, the first antigen binding domain specifically binds a tumor-specific antigen or a tumor-associated antigen. In some embodiments, the first antigen binding domain specifically binds an antigen from the group: glioma-associated antigen, carcinoembryonic antigen (CEA), beta-human chorionic gonadotropin, alpha-fetoprotein (AFP), lectin-reactive AFP, thyroglobulin, RAGE-1, MN-CA IX, human telomerase reverse transcriptase, RU1, RU2 (AS), intestinal carboxyl esterase, mut HSP70-2, M-CSF, prostate-specific antigen (PSA), PAP, NY-ESO-1, LAGE-1a, p53, prostein, PSMA, HER2, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, GD2, GD3, B7-H3, GPC2, L1CAM, EGFR, mesothelin, MART-1, gp100 (Pmel 17), tyrosinase, TRP-1, TRP-2, MAGE-1, MAGE-3, BAGE, GAGE-1, GAGE-2, p15, CEA, p53, Ras, HER-2, BCR-ABL, E2A-PRL, H4-RET, IGH-IGK, MYL-RAR, EBVA, human papillomavirus (HPV) antigens E6 and E7, TSP-180, MAGE-4, MAGE-5, MAGE-6, RAGE, p185erbB2, p180erbB-3, c-met, nm-23H1, PSA, TAG-72, CA 19-9, CA 72-4, CAM 17.1, NuMa, K-ras, β-Catenin, CDK4, Mum-1, p15, p16, 43-9F, 5T4, 791Tgp72, α-fetoprotein, R-HCG, BCA225, BTAA, CA125, BCAA, CA195, CA242, CA-50, CAM43, CD68/P1, CO-029, FGF-5, G250, Ga733/EpCAM, HTgp-175, M344, MA-50, MG7-Ag, MOV18, NB/70K, NY-CO-1, RCAS1, SDCCAG16, TA-90, TAAL6, TAG72, TLP, TPS CD19, CD20, CD22, ROR1, and GD2.

Another aspect of the disclosure is a chimeric polypeptide for co-stimulating an immune receptor, such as a CAR and/or a TCR, wherein the CAR having a first antigen binding domain specific for a first antigen, the TCR having a second antigen binding domain, the chimeric polypeptide having a third antigen binding domain, where the chimeric polypeptide comprises in order from N-terminal to C-terminal: the first antigen binding domain specific for the first antigen, the second antigen binding domain specific for the second antigen and third antigen binding domain specific for the third antigen; a spacer domain; a transmembrane domain; and a cytoplasmic signaling domain, wherein the cytoplasmic signaling domain comprises a CD2 signaling domain, and does not comprise a CD3ζ activating domain, and wherein the second antigen is not CD58. In some embodiments, the first antigen, the second antigen, and the third antigen are different from each other. In some embodiments, at least two of the first antigen, the second antigen, and the third antigen are the same.

In some embodiments, the second antigen is a tumor-specific antigen, or a tumor-associated antigen. In some embodiments, the second antigen is selected from the group consisting of glioma-associated antigen, carcinoembryonic antigen (CEA), beta-human chorionic gonadotropin, alpha-fetoprotein (AFP), lectin-reactive AFP, thyroglobulin, RAGE-1, MN-CA IX, human telomerase reverse transcriptase, RU1, RU2 (AS), intestinal carboxyl esterase, mut HSP70-2, M-CSF, prostate-specific antigen (PSA), PAP, NY-ESO-1, LAGE-1a, p53, prostein, PSMA, HER2, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, GD2, GD3, B7-H3, GPC2, L1CAM, EGFR, mesothelin, MART-1, gp100 (Pmel 17), tyrosinase, TRP-1, TRP-2, MAGE-1, MAGE-3, BAGE, GAGE-1, GAGE-2, p15, p53, Ras, HER-2, BCR-ABL, E2A-PRL, H4-RET, IGH-IGK, MYL-RAR, EBVA, human papillomavirus (HPV) antigens E6 and E7, TSP-180, MAGE-4, MAGE-5, MAGE-6, RAGE, p185erbB2, p180erbB-3, c-met, nm-23H1, PSA, TAG-72, CA 19-9, CA 72-4, CAM 17.1, NuMa, K-ras, β-Catenin, CDK4, Mum-1, p15, p16, 43-9F, 5T4, 791Tgp72, α-fetoprotein, β-HCG, BCA225, BTAA, CA125, BCAA, CA195, CA242, CA-50, CAM43, CD68/P1, CO-029, FGF-5, G250, Ga733/EpCAM, HTgp-175, M344, MA-50, MG7-Ag, MOV18, NB/70K, NY-CO-1, RCAS1, SDCCAG16, TA-90, TAAL6, TAG72, TLP, TPS CD19, CD20, CD22, ROR1, and GD2. In some embodiments, the second antigen is selected from the group consisting of B7H3, BAFF-R, CD19, CD20, CD22, GD2, GD3, GPC2, IL13Rα2, and ROR1.

In some embodiments, the third antigen is a tumor-specific antigen, or a tumor-associated antigen. In some embodiments, the third antigen is selected from the group consisting of glioma-associated antigen, carcinoembryonic antigen (CEA), beta-human chorionic gonadotropin, alpha-fetoprotein (AFP), lectin-reactive AFP, thyroglobulin, RAGE-1, MN-CA IX, human telomerase reverse transcriptase, RU1, RU2 (AS), intestinal carboxyl esterase, mut HSP70-2, M-CSF, prostate-specific antigen (PSA), PAP, NY-ESO-1, LAGE-1a, p53, prostein, PSMA, HER2, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, GD2, GD3, B7-H3, GPC2, L1CAM, EGFR, mesothelin, MART-1, gp100 (Pmel 17), tyrosinase, TRP-1, TRP-2, MAGE-1, MAGE-3, BAGE, GAGE-1, GAGE-2, p15, p53, Ras, HER-2, BCR-ABL, E2A-PRL, H4-RET, IGH-IGK, MYL-RAR, EBVA, human papillomavirus (HPV) antigens E6 and E7, TSP-180, MAGE-4, MAGE-5, MAGE-6, RAGE, p185erbB2, p180erbB-3, c-met, nm-23H1, PSA, TAG-72, CA 19-9, CA 72-4, CAM 17.1, NuMa, K-ras, β-Catenin, CDK4, Mum-1, p15, p16, 43-9F, 5T4, 791Tgp72, α-fetoprotein, β-HCG, BCA225, BTAA, CA125, BCAA, CA195, CA242, CA-50, CAM43, CD68/P1, CO-029, FGF-5, G250, Ga733/EpCAM, HTgp-175, M344, MA-50, MG7-Ag, MOV18, NB/70K, NY-CO-1, RCAS1, SDCCAG16, TA-90, TAAL6, TAG72, TLP, TPS CD19, CD20, CD22, ROR1, and GD2. In some embodiments, the second antigen is selected from the group consisting of B7H3, BAFF-R, CD19, CD20, CD22, GD2, GD3, GPC2, ILI3Rα2, and ROR1.

In some embodiments, CD2 signaling is enhanced with a transgenic T-cell receptor (TCR). In some embodiments, the transgenic TCR comprises a second antigen binding domain specific for a second antigen. In some embodiments, the transgenic TCR comprises a transmembrane domain. In some embodiments, the transmembrane domain is selected from the group consisting of all or part of the transmembrane domain of the CD3 zeta chain (CD3ζ), CD2, CD8α, CD28, CD40, CTLA4, OX40, PD-1, 4-1BB (CD137), FcERIγ, ICOS (CD278), ILRB (CD122), CTLA-4, and PD-1, and IL-2RG (CD132). In some embodiments, the transgenic TCR further comprises a cytoplasmic signaling domain. In some embodiments, the second antigen of the transgenic TCR is a tumor-specific antigen or a tumor-associated antigen presented by MHC class I or MHC class II.

In some embodiments, the chimeric polypeptide has a cytoplasmic signaling domain comprises an additional co-stimulating signaling domain. In some embodiments, the additional co-stimulating signaling domain comprises a 4-1BB signaling domain, a CD27 signaling domain, a CD28 signaling domain, or an OX40 signaling domain. In some embodiments, the additional co-stimulating signaling domain comprises a 4-1BB signaling domain, a CD27 signaling domain, or an OX40 signaling domain. In some embodiments, the additional co-stimulating signaling domain comprises a 4-1BB signaling domain. In some embodiments, the additional co-stimulating signaling domain comprises a toll-like receptor signaling domain.

In some embodiments, the chimeric polypeptide has an amino acid sequence that is at least 70%, 80%, 90%, 95%, 97%, 98%, 99%, or about 100% identical to the sequence of one of SEQ ID NOs: 20 to 29 and 92-112. In some embodiments, the transmembrane domain is derived from CD8α, CD2, or CD28. In some embodiments, the transmembrane domain is a CD28 transmembrane domain. In some embodiments, the transmembrane domain is a CD8a transmembrane domain. In some embodiments, the transmembrane domain is a CD2 transmembrane domain.

Another aspect of the disclosure is a nucleic acid that encodes the CAR, the chimeric polypeptide described above, and/or the TCR. In some embodiments, the nucleic acid encodes a polypeptide that has a sequence that is at least 70%, 80%, 90%, 95%, 97%, 98%, 99%, or about 100% identical to the sequence of one of SEQ ID NOs: 41 to 58. In some embodiments, the nucleic acid encodes a chimeric polypeptide. In some embodiments, the nucleic acid encodes a polypeptide that has a sequence that is at least about 70%, 80%, 90%, 95%, 97%, 98%, 99%, or about 100% identical to the sequence of one of SEQ ID NOs: 49 to 58.

In some embodiments, the nucleic acid encodes a chimeric polypeptide, a CAR and/or a TCR. In some embodiments, the nucleic acid encodes a polypeptide that has a sequence that is at least 70%, 80%, 90%, 95%, 97%, 98%, 99%, or about 100% identical to the sequence of one of SEQ ID NOs: 72, 74, 76, 78, 82, 84, or 86. In some embodiments, the CAR antigen binding domain and the chimeric polypeptide antigen binding domain are specific for different antigens. In some embodiments, the CAR antigen binding domain and the chimeric polypeptide antigen binding domain are specific for the same antigen. In some embodiments, the first binding domain and the second binding domain are specific for different epitopes of the same antigen. In some embodiments, the nucleic acid encoding a chimeric polypeptide and the nucleic acid encoding a CAR each have an individual promoter. In some embodiments, the nucleic acid encoding a chimeric polypeptide and the nucleic acid encoding a CAR are separated by a ribosomal re-entry site. In some embodiments, the chimeric polypeptide and the CAR are encoded as a single polypeptide, where the chimeric polypeptide and the CAR are separated by a self-cleaving peptide. In some embodiments, the self-cleaving peptide is a 2A peptide. In some embodiments, the CAR transmembrane domain and the chimeric polypeptide transmembrane domain are different. In some embodiments, the nucleic acid comprises DNA. In some embodiments, the nucleic acid comprises RNA.

Another aspect of the disclosure is a vector, comprising any of the nucleic acids set forth above operably linked to a promoter. In some embodiments, the vector is a lentiviral vector.

Another aspect of the disclosure is an engineered cell, comprising the CAR, chimeric polypeptide, and/or the TCR, nucleic acid, and/or vector described herein. In some embodiments, the cell expresses a CAR having a CD2 co-stimulatory domain, as described herein. In some embodiments, the cell expresses a chimeric polypeptide described herein. In some embodiments, the cell expresses a transgenic TCR described herein.

In some embodiments, the cell expresses a CAR specific for a first antigen and a chimeric polypeptide specific for a second antigen and a TCR specific for a third antigen. In some embodiments, the first antigen and the second antigen are different from each other. In some embodiments, the first antigen and the second antigen are the same. In some embodiments, the first binding domain and the second binding domain are specific for different epitopes of the same antigen. In some embodiments, the CAR transmembrane domain is different from the chimeric polypeptide transmembrane domain. In some embodiments, the first antigen, the second antigen, and the third antigen are different from each other. In some embodiments, at least two of the first antigen, the second antigen, and the third antigen are the same.

Another aspect of the disclosure is a method for making an engineered cell, by providing an immune cell, and transducing the immune cell with a nucleic acid that encodes a chimeric polypeptide described above. In some embodiments, the immune cell is a T cell, NK cell, NKT cell, a tumor-inflitrating lymphocyte (TIL), or a macrophage. In some embodiments, the immune cell is a precursor cell of a T cell, NK cell, NKT cell, a tumor-inflitrating lymphocyte (TIL), or a macrophage. In some embodiments, the immune cell is further transduced with a nucleic acid that encodes a CAR.

Another aspect of the disclosure is a method for making a CAR-T cell having improved functional characteristics, by providing an immune cell, and transducing the immune cell with a nucleic acid that encodes a CAR, and a nucleic acid that encodes a chimeric polypeptide, and/or a TCR to produce a CAR-T cell having a CAR specific for a target cell having a first antigen, the CAR-T cell further comprising a chimeric polypeptide; wherein the functional characteristic is: (i) efficacy against target cells that downregulate expression of or do not substantially express CD58; (ii) efficacy against target cells that downregulate or the selected antigen, or express a mutated form of the selected antigen; (iii) improved selectivity for the target cell; or (iv) rescue a loss of a CAR target antigen. In some embodiments, the chimeric polypeptide comprises an antigen binding domain specific for an antigen expressed by the target cell. In some embodiments, the transgenic TCR includes an antigen binding domain specific for an antigen expressed by the target cell. In some embodiments, the CAR target antigen that is lost is CD19.

Another aspect of the disclosure is a method for aiding in the treatment of a subject having a hyperproliferative disorder characterized by the proliferation of a target cell having at least a first antigen, by providing an engineered cell that expresses a CAR having a CD2 co-stimulatory domain and at least one additional co-stimulatory domain, or expresses a CAR, a chimeric polypeptide, and/or a TCR; and administering a therapeutically effective number of the engineered cells, wherein the engineered cells aid in the treatment of the subject. In some embodiments, the CAR has at least two co-stimulatory domains in addition to a CD2 co-stimulatory domain, or the CAR has at least two co-stimulatory domains and is expressed with a chimeric polypeptide. In some embodiments, wherein the method further includes determining the degree of functional CD58 expression by the target cell. In some embodiments, the method further includes providing a determination of CD58 expression by the target cell prior to administering the engineered cell, and administering a therapeutically effective number of the engineered cells if the determination of CD58 expression indicates that the target cell expresses mutated CD58, or expresses CD58 at a level below a threshold level. In some embodiments, the threshold level is about 50,000, 45,000, 40,000, 35,000, 30,000, 25,000, 20,000, 15,000, 14,000, 13,000, 12,000, 11,000, 10,000, 9,000, 8,000, 7,000, 6,000, 5,000, 4,000, 3,000, 2,000, 1,000, or 500 CD58 molecules per target cell. In some embodiments, the method further provides an antibody or an antigen binding fragment thereof. In some embodiments, the antibody or the antigen binding fragment thereof is capable of stimulating CD2 signalling in the target cell microenvironment. In some embodiments, the antigody is a multispecific antibody. In some embodiments, the multispecific antibody is specific for an anti-tumor antigen, CD2, and/or CD3. In some embodiments, the antibody binding fragment is selected from the group consisting of scFv, scFv-Fc, Fab, Fab′, (Fab)₂, (Fab′)₂, minibody, diabody, triabody, and dAb. In some embodiments, the antibody binding fragment is specific for an anti-tumor antigen, CD2, and/or CD3. In some embodiments, the method further provides a therapeutic agent capable of crosslinking native CD2 in response to a tumor specific antigen expressed in the target cell microenvironment. In some embodiments, the therapeutic agent is secreted or cell-surface expressed. In some embodiments, the therapeutic agent is anti-CD2 scFv, an antibody, a Fab, DARPIN, a ligand, or an antigen binding domain.

Another aspect of the disclosure is a system for aiding in the treatment of a subject having a hyperproliferative disorder characterized by the proliferation of a target cell having at least a first antigen, wherein the system comprises an engineered cell of the disclosure having a CAR that is specific for a first antigen, and a labeled binding agent specific for CD58. In some embodiments, the CAR comprises a cytoplasmic signaling domain, wherein the cytoplasmic signaling domain comprises a 4-1BB signaling domain, a CD2 signaling domain, and a CD3ζ activating domain. In some embodiments, the CAR comprises a cytoplasmic signaling domain, wherein the cytoplasmic signaling domain comprises a co-signaling domain and a CD3ζ activating domain, and the engineered cell further comprises a chimeric polypeptide of the disclosure. In some embodiments, the labeled binding agent comprises an anti-CD58 antibody or antibody derivative. In some embodiments, the engineered cell further comprises a transgenic TCR described herein.

Another aspect of the disclosure is a method for aiding in the treatment of a subject having a hyperproliferative disorder characterized by the proliferation of a target cell having at least a first antigen, and wherein the target cells express a reduced level of CD58 and/or express a form of CD58 that has reduced ability to activate CD2, by: providing an engineered cell, wherein the engineered cell expresses a CAR that comprises a first antigen binding domain capable of specifically binding the first antigen, a spacer domain, a transmembrane domain, and a cytoplasmic signaling domain, wherein the cytoplasmic signaling domain comprises a CD2 signaling domain, and a CD3ζ activating domain; determining the expression level of functional CD58 in a sample obtained from the subject, wherein the sample contains a tumor cell; and administering a therapeutically effective number of engineered cells if the expression level of functional CD58 is less than a predetermined threshold level. In some embodiments, the cytoplasmic signaling domain comprises an additional co-stimulatory domain. In some embodiments, the additional co-stimulatory domain comprises a 4-1BB signaling domain, a CD27 signaling domain, a CD28 signaling domain, or an OX40 signaling domain. In some embodiments, the additional signaling domain comprises a 4-1BB signaling domain, a CD27 signaling domain, or an OX40 signaling domain. In some embodiments, the additional co-stimulatory domain comprises a 4-1BB signaling domain. In some embodiments, the threshold level is about 50,000, 45,000, 40,000, 35,000, 30,000, 25,000, 20,000, 15,000, 14,000, 13,000, 12,000, 11,000, 10,000, 9,000, 8,000, 7,000, 6,000, 5,000, 4,000, 3,000, 2,000, 1,000, or 500 functional CD58 molecules per target cell.

Another aspect of the disclosure is a therapeutic composition, comprising: a nucleic acid of the disclosure, a vector of the disclosure, and/or an engineered cell of the disclosure; and a pharmaceutically acceptable carrier.

Another aspect of the disclosure is the use for the treatment of disease in a human of a CAR having a CD2 co-stimulating signaling domain, chimeric polypeptide of the disclosure, a transgenic TCR of the disclosure, a nucleic acid of the disclosure, a vector of the disclosure, an engineered cell of the disclosure, and/or a pharmaceutical composition of the disclosure. In some embodiments, the disease is a hyperproliferative disorder. In some embodiments, the disease is cancer.

Another aspect of the disclosure is the use for the manufacture of a medicament for the treatment of disease of a CAR having a CD2 co-stimulating signaling domain, chimeric polypeptide of the disclosure, a nucleic acid of the disclosure, a vector of the disclosure, an engineered cell of the disclosure, and/or a pharmaceutical composition of the disclosure.

Another aspect of the disclosure is a nucleic acid comprising a polynucleotide encoding a chimeric signaling molecule comprising: (i) a cytoplasmic signaling domain, wherein the cytoplasmic signaling domain comprises a CD2 signaling domain but does not comprise a CD3-zeta domain; and at least one of: (ii) an antigen binding domain; or (iii) a transmembrane domain. In some embodiments, the cytoplasmic signaling domain further comprises a co-stimulating domain. In some embodiments, the co-stimulating domain comprises at least one of: a 4-1BB signaling domain, a CD27 signaling domain, an OX40 signaling domain, a CD28 signaling domain, a CD278 signaling domain, a CD40 signaling domain, a CD40L signaling domain, a toll-like receptor signaling domain, or any combination thereof. In some embodiments, the cytoplasmic signaling domain comprises an amino acid sequence that is at least 80% homologous to the amino acid sequence of SEQ ID NO: 8. In some embodiments, the cytoplasmic domain consists of SEQ ID NO: 8. In some embodiments, chimeric signaling molecule comprises an amino acid sequence that is at least about 80% homologous to an amino acid sequence selected from the group consisting of: SEQ ID NOs: 20 to 29 and 92-112. In some embodiments, the nucleic acid can comprise the polynucleotide encoding the chimeric signaling molecule, wherein the chimeric signaling molecule comprises (ii) the antigen binding domain. In some embodiments, the antigen binding domain comprises an antibody or an antigen binding fragment thereof. In some embodiments, the antigen binding domain comprises an scFv, an sdAb, an Fab, a bispecific antibody or an antigen binding fragment thereof, a trispecific antibody or an antigen binding fragment thereof, a bispecific diabody, a trispecific traibody, an scFv-Fc, a minibody, a VhH domain, an hcIgG domain, a V-NAR domain, or any combination thereof. In some embodiments, the antigen binding domain is specific for a B cell surface antigen. In some embodiments, the B cell surface antigen is selected from the group consisting of HLA-DR, CD20, CD32b, CD37, CD38, CD52, CD81, CD79A, CD79B, CD138, CSI, GPRC5D, a BAFF receptor, APRIL, BCMA, and TAC. In some embodiments, the antigen binding domain is specific for a tumor associated antigen. In some embodiments, the tumor associated antigen is selected from the group consisting of HLA-DR, CD20, CD32b, CD37, CD38, CD52, CD81, CD79A, CD79B, CD138, CSI, GPRC5D, a BAFF receptor, APRIL, BCMA, TACI, glioma-associated antigen, carcinoembryonic antigen (CEA), beta-human chorionic gonadotropin, alpha-fetoprotein (AFP), lectin-reactive AFP, thyroglobulin, RAGE-1, MN-CA IX, human telomerase reverse transcriptase, RU1, RU2 (AS), intestinal carboxyl esterase, mut HSP70-2, M-CSF, prostate-specific antigen (PSA), PAP, NY-ESO-1, LAGE-1a, p53, prostein, PSMA, HER2, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, GD2, GD3, B7-H3, GPC2, L1CAM, EGFR, mesothelin, MART-1, gp100 (Pmel 17), tyrosinase, TRP-1, TRP-2, MAGE-1, MAGE-3, BAGE, GAGE-1, GAGE-2, p15, p53, Ras, HER-2, BCR-ABL, E2A-PRL, H4-RET, IGH-IGK, MYL-RAR, EBVA, human papillomavirus (HPV) antigens E6 and E7, TSP-180, MAGE-4, MAGE-5, MAGE-6, RAGE, p185erbB2, p180erbB-3, c-met, nm-23H1, PSA, TAG-72, CA 19-9, CA 72-4, CAM 17.1, NuMa, K-ras, β-Catenin, CDK4, Mum-1, p15, p16, 43-9F, 5T4, 791Tgp72, α-fetoprotein, β-HCG, BCA225, BTAA, CA125, BCAA, CA195, CA242, CA-50, CAM43, CD68/P1, CO-029, FGF-5, G250, Ga733/EpCAM, HTgp-175, M344, MA-50, MG7-Ag, MOV18, NB/70K, NY-CO-1, RCAS1, SDCCAGI6, TA-90, TAAL6, TAG72, TLP, TPS CD19, CD20, CD22, ROR1, and GD2. In some embodiments, the nucleic acid comprises the polynucleotide encoding the chimeric signaling molecule, wherein the chimeric signaling molecule comprises (iii) the transmembrane domain. In some embodiments, the transmembrane domain is selected from the group consisting of all or part of the transmembrane domain of CD3ζ, CD2, CD8a, CD28, CD40, CTLA4, OX40, PD-1, 4-1BB (CD137), FcERIγ, ICOS (CD278), ILRB (CD122), and IL-2RG (CD132).

Another aspect of the disclosre is a nucleic acid comprising a polynucleotide encoding a chimeric signaling molecule comprising a sequence that is at least about 80% homologous to a sequence selected from the group consisting of SEQ ID NOs: 20 to 29 and 92-112.

Another aspect of the disclosure is a nucleic acid composition comprising a nucleic acid comprising a polynucleotide encoding a chimeric signaling molecule according to any one of claims 1-14, and further, a nucleic acid comprising a polynucleotide encoding an additional polypeptide. In some embodiments, the additional polypeptide comprises a chimeric antigen receptor (CAR), a T cell receptor (TCR), or a TCR-CAR. In some embodiments, the CAR comprises a first generation CAR, second generation CAR, or a third generation CAR. In some embodiments, the additional polypeptide comprises a CAR comprising a CD3-zeta domain. In some embodiments, the additional polypeptide comprises a CAR comprising an antigen binding domain. In some embodiments, the antigen binding domain comprises an antibody or an antigen binding fragment thereof. In some embodiments, the antigen binding domain comprises an scFv, an sdAb, an Fab, a bispecific antibody or an antigen binding fragment thereof, a trispecific antibody or an antigen binding fragment thereof, a bispecific diabody, a trispecific traibody, an scFv-Fc, a minibody, a VhH domain, an hcIgG domain, a V-NAR domain, or any combination thereof. In some embodiments, the antigen binding domain is specific for a B cell surface antigen. In some embodiments, the B cell surface antigen is selected from the group consisting of HLA-DR, CD20, CD32b, CD37, CD38, CD52, CD81, CD79A, CD79B, CD138, CSI, GPRC5D, a BAFF receptor, APRIL, BCMA, and TACI. In some embodiments, the antigen binding domain is specific for a tumor associated antigen. In some embodiments, the tumor associated antigen is selected from the group consisting of glioma-associated antigen, carcinoembryonic antigen (CEA), beta-human chorionic gonadotropin, alpha-fetoprotein (AFP), lectin-reactive AFP, thyroglobulin, RAGE-1, MN-CA IX, human telomerase reverse transcriptase, RU1, RU2 (AS), intestinal carboxyl esterase, mut HSP70-2, M-CSF, prostate-specific antigen (PSA), PAP, NY-ESO-1, LAGE-1a, p53, prostein, PSMA, HER2, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, GD2, GD3, B7-H3, GPC2, L1CAM, EGFR, mesothelin, MART-1, gp100 (Pmel 17), tyrosinase, TRP-1, TRP-2, MAGE-1, MAGE-3, BAGE, GAGE-1, GAGE-2, p15, p53, Ras, HER-2, BCR-ABL, E2A-PRL, H4-RET, IGH-IGK, MYL-RAR, EBVA, human papillomavirus (HPV) antigens E6 and E7, TSP-180, MAGE-4, MAGE-5, MAGE-6, RAGE, p185erbB2, p180erbB-3, c-met, nm-23H1, PSA, TAG-72, CA 19-9, CA 72-4, CAM 17.1, NuMa, K-ras, β-Catenin, CDK4, Mum-1, p15, p16, 43-9F, 5T4, 791Tgp72, α-fetoprotein, β-HCG, BCA225, BTAA, CA125, BCAA, CA195, CA242, CA-50, CAM43, CD68/P1, CO-029, FGF-5, G250, Ga733/EpCAM, HTgp-175, M344, MA-50, MG7-Ag, MOV18, NB/70K, NY-CO-1, RCAS1, SDCCAG16, TA-90, TAAL6, TAG72, TLP, TPS CD19, CD20, CD22, ROR1, and GD2. In some embodiments, the additional polypeptide further comprises a co-stimulating domain. In some embodiments, the co-stimulating domain comprises at least one of: a 4-1BB signaling domain, a CD27 signaling domain, an OX40 signaling domain, a CD28 signaling domain, a CD278 signaling domain, a CD40 signaling domain, a CD40L signaling domain, a toll-like receptor signaling domain, or any combination thereof. In some embodiments, the additional polypeptide further comprises the transmembrane domain. In some embodiments, the transmembrane domain is selected from the group consisting of all or part of the transmembrane domain of CD3ζ, CD2, CD8α, CD28, CD40, CTLA4, OX40, PD-1, 4-1BB (CD137), FcERIγ, ICOS (CD278), ILRB (CD122), and IL-2RG (CD132).

Another aspect of the disclosure is a vector comprising a nucleic acid provided herein or a nucleic acid composition provided herein.

Another aspect of the disclosure is a cell comprising a vector provided herein. In some embodiments, the cell is an immune cell, a stem cell, a mammalian cell, a primate cell, or a human cell. In some embodiments, the cell is autologous or allogeneic. In some embodiments, the cell is a T cell, a CD8-positive T cell, a CD4-positive T cell, a regulatory T cell, a cytotoxic T cell, or a tumor infiltrating lymphocyte.

Another aspect of the disclosure is a method treating of a subject having a hyperproliferative disorder, the method including: administering to the subject a composition comprising a therapeutically effective number of the cell described herein. Another aspect of the disclosure is a method treating of a subject having a hyperproliferative disorder characterized by proliferation of a target cell that (i) lacks expression of CD58; (ii) expresses a reduced level of CD58; or (iii) expresses a form of CD58 that has reduced ability to activate a CD2, the method comprising: administering to the subject a composition comprising a therapeutically effective number of the cell described herein.

Another aspect of the disclosure is a chimeric polypeptide comprising: i) an antigen binding domain; ii) a transmembrane domain; iii) a cytosplasim signaling domain comprising a CD2 signaling domain and a co-stimulatory domain, wherein the antigen binding domain comprises an amino acid sequence that is at least about 80% homologous to a sequence selected from the group consisiting of SEQ ID NOs: 1, 2, 3, and 4; the transmembrane domain comprises an amino acid sequence that is at least about 80% homologous to the sequence selected from the group consisting of SEQ ID NOs: 5, 6, and 7; the CD2 signaling domain comprises an amino acid sequence that is at least about 80% homologous to the sequence of SEQ ID NO: 8 and the co-stimulatory domain comprises an amino acid sequence that is at least about 80% homologous to a sequence selected from the group consisting of: SEQ ID NOs: 10 and 11. In some embodiments, the cytoplasmic sinaling domain does not comprise a CD3-zeta activating domain. In some embodiments, the cytoplasmic sinaling domain comprises an activating domain, wherein the activating domain comprises an amino acid sequence that is at least about 80% homologous to the sequence of SEQ ID NO: 9.

### BRIEF DESCRIPTION OF THE DRAWINGS

FIG. 1 shows the effect of knocking out CD58 expression in Nalm6 target cells. As described in Example 1, knocking out CD58 expression significantly reduced the production of IFNγ and IL-2 in those CAR T cells that produced measurable amounts with the control target. In each panel, the three pairs of bars represent CARs CD19-28z, CD19-BBz, and CD22-BBz, from left to right.

FIG. 2 shows the effect of knocking out CD58 expression in Nalm6 target cells, as measured by target cell killing by CD22 CARs, where the target cells are either CD58⁺ or CD58⁻, as described in Example 2.

FIG. 3 shows the effect of knocking out CD58 expression in Nalm6 target cells, as measured by target cell killing by CD19 CARs, where the target cells are either CD58⁺ or CD58⁻, as described in Example 3.

FIG. 4 shows the effect of knocking out CD58 expression in Nalm6 target cells, as measured by target cell killing by CD19 CARs, where the target cells are either CD58⁺ or CD58⁻, and the target antigen (CD19) expression is reduced from 45,000 copies per cell to about 6,196 copies per cell, as described in Example 3.

FIG. 5 shows the effect of knocking out CD58 expression in Nalm6 target cells, as measured by target cell killing by CD19 CARs, where the target cells are either CD58⁺ or CD58⁻, and CD19 expression is further reduced to about 963 copies per cell, as described in Example 3.

FIG. 6 shows that CD22-BBz CAR T cells can control growth of CD58⁺ Nalm6 cells in vivo, but fail to achieve more than a transient response with CD58⁻ Nalm6 cells, as described in Example 4.

FIG. 7 shows that CD19-28z CAR T cells were able to control tumor growth of CD58⁺ cells in vivo, but achieved only a transient response with CD58⁻ cells, as described in Example 5.

FIG. 8 shows that CD19-BBz CAR T cells were able to control tumor growth of CD58⁺ cells in vivo, but achieved only a transient response with CD58⁻ cells, as described in Example 5.

FIG. 9 shows that CD2-containing CARs (other than m971-BB-z-CD2) outperformed m971-BBz in cytotoxicity against both CD58⁺ and CD58⁻ cells, as described in Example 6.

FIG. 10 shows that CD2-containing CARs (other than m971-BB-z-CD2) outperformed m971-BBz in cytokine release against CD58⁻ cells, as described in Example 6.

In each panel, no cytokine release was observed in the absence of tumor target cells. In each panel, the middle cluster of bars indicates cytokine expression in the presence of CD58⁺ Nalm6 target cells, while the cluster of bars to the right indicates expression in the presence of CD58⁻ Nalm6 target cells (“N6 CD58KO”). In the middle clusters of bars, the CARs are m971-BBz, m971-CD2-BBz, m971-CD2z, and m971-BB-CD2z, from left to right. No expression was observed for m971-BBz-CD2, or mock-transduced T cells. In the clusters of bars in the right of each panel, the CARs are m971-CD2-BBz, m971-CD2z, and m971-BB-CD2z, from left to right. No expression was observed for m971-BBz, m971-BBz-CD2, or mock-transduced T cells.

FIG. 11 shows that m971-CD2-BBz CAR-T cells demonstrated enhanced tumor control and enhanced survival compared to m971-BBz when administered to mice inoculated with CD58− tumor cells, as described in Example 7. The left panel shows flux value from luciferase-expressing tumor cells in mice. The upper trace indicates mice treated with mock-transduced T cells; the middle trace indicates mice treated with m971-BBz CAR T cells, and the lower trace indicates mice treated with m971-CD2-BBz CAR T cells. In the right panel, percent survival is shown, indicating that no mice treated with mock-transduced T cells survived to day 20; all mice treated with m971-BBz CAR T cells survived past day 20, but none survived to day 25; and all mice treated with m971-CD2-BBz CAR T cells survived past day 30, some surviving as long as day 45.

FIG. 12 shows that CD19 CAR-T cells with or without a CD2 co-stimulatory domain performed comparably in terms of cytotoxicity against both CD58⁺ and CD58⁻ cells when the target cells express about 45,000 CD19 molecules per cell, as described in Example 7.

FIG. 13 shows that CD19 CD2 CAR-T outperformed CD19-BBz CAR-T in terms of cytotoxicity against CD58⁻ cells when the target cells express about 6,196 CD19 molecules per cell, as described in Example 7. This degree of CD19 expression provides a closer approximation of CD19 expression levels found in lymphoma.

FIG. 14 shows that CD19 CD2 CARs also outperformed CD19-BBz cells in terms of cytokine release against both CD58⁺ and CD58⁻ cells when incubated for 24 hours, as described in Example 7. In each panel, no cytokine release was observed in the absence of tumor target cells. In each panel, the middle cluster of bars indicates cytokine expression in the presence of CD58⁺ Nalm6 target cells, while the cluster of bars to the right indicates expression in the presence of CD58⁻ Nalm6 target cells (“N6 CD58KO”). In the middle clusters of bars, the CARs are CD19-BBz, CD19-CD2z, and CD19-BB-CD2z, from left to right. No expression was observed for mock-transduced T cells. In the clusters of bars in the right of each panel, the CARs are CD19-BBz, CD19-CD2z, and CD19-BB-CD2z, from left to right. IL-2 expression in CD19-BBz was barely detectable in the presence of CD58⁻ cells. Again, no expression was observed for mock-transduced T cells.

FIG. 15 shows that adding a CD2 co-stimulatory domain to the CD28 co-stimulatory domain decreased IL-2 and IFNγ release significantly, as described in Example 8. In each panel, no cytokine release was observed in the absence of tumor target cells. In each panel, the middle cluster of bars indicates cytokine expression in the presence of CD58⁺ Nalm6 target cells, while the cluster of bars to the right indicates expression in the presence of CD58⁻ Nalm6 target cells (“N6 CD58KO”). In the middle clusters of bars, the CARs are m971-28z and m971-CD2-28z, from left to right. No expression was observed for mock-transduced T cells. In the presence of CD58⁻ Nalm6 target cells, no IL-2 expression was detected for either receptor, and no IFNγ expression was detected for m971-CD2-28z.

FIG. 16 shows that m971-28z performed better than m971-CD2-28z in terms of cytotoxicity, as described in Example 8.

FIG. 17 shows that adding a CD2 co-stimulatory domain to the 4-1BB co-stimulatory domain improved the CAR cytokine release against CD58KO cells, and for CD19 CARs adding CD2 improved cytokine release also against CD58⁺ cells, as described in Example 8. In each panel, the middle cluster of bars indicates cytokine expression in the presence of CD58⁺ Nalm6 target cells, while the cluster of bars to the right indicates expression in the presence of CD58⁻ Nalm6 target cells (“N6 CD58KO”). In the middle clusters of bars in the left panel, the CARs are m971-BBz, and m971-BB-CD2z, from left to right. The left panel shows that no IL-2 expression by m971-BBz CAR-T cells was observed in the absence of CD58⁺ cells. In the right panel, the CARs are CD19-BBz, and CD19-BB-CD2z, from left to right. This panel shows that no IL-2 expression was observed for CD19-BBz in the absence of CD58⁺ cells.

FIG. 18 shows that CD19-CD2-BBz performed better than CD19-BBz in terms of cytotoxicity, particularly against CD58KO cells, as described in Example 8.

FIG. 19 illustrates schematically the difference between an exemplary CAR and a CD2-trans chimeric polypeptide. The left panel depicts a schematic structure for a CD2 CAR, having a CD2 signaling domain and a co-stimulatory domain, in addition to a CD3ζ activating domain. The right panel depicts a second generation CAR having a co-stimulatory domain, and a chimeric polypeptide having an antigen binding domain and a CD2 signaling domain. Note that the chimeric polypeptide lacks a CD3ζ activating domain, and thus acts only in trans to co-stimulate the CAR.

FIG. 20 shows that CAR-T cells having a chimeric polypeptide in addition to a CAR outperformed CAR-T cells without a chimeric polypeptide in cytotoxicity against CD58KO cells, as described in Example 9.

FIG. 21 CAR-T cells having a chimeric polypeptide in addition to a CAR outperformed CAR-T cells without a chimeric polypeptide in cytokine release against CD58KO cells, as described in Example 9. In each panel, the middle cluster of bars indicates cytokine expression in the presence of CD58⁺ Nalm6 target cells, while the cluster of bars to the right indicates expression in the presence of CD58⁻ Nalm6 target cells (“N6 CD58KO”). In the middle clusters of bars in the left panel, the CARs are m971-BBz+CD19-28tm-CD2 (“trans receptor”, construct m971-BBz-2A-CD19-28tm-CD2) and m971-BBz, from left to right. The left panel shows that no IL-2 expression by m971-BBz CAR-T cells was observed in the absence of CD58⁺ cells; only expression by m971-BBz+CD19-28tm-CD2 was observed. In the right panel (IFNγ expression), the CARs (with and without CD58) are m971-BBz+CD19-28tm-CD2 (from construct m971-BBz-2A-CD19-2tm-CD2), m971-BBz+CD19-8tm-CD2 (from construct m971-BBz-2A-CD19-8tm-CD2), m971-BBz+CD19-2tm-CD2 (from construct m971-BBz-2A-CD19-2tm-CD2), m971-BBz+CD19-28m (control, from construct m971-BBz-2A-CD19-28m-STOP*), and m971-BBz (control, from construct m971-BBz-2A-STOP*), from left to right.

FIG. 22 shows that a trans construct (m971-BBz-2A-CD19-28tm-CD2) performed better in cytotoxicity than two cis constructs, as described in Example 10.

FIG. 23 shows that a trans construct (m971-BBz-2A-CD19-28tm-CD2) performed better in cytokine release than two cis constructs, as described in Example 10. In each panel, the CARs are m971-CD2z, m971-BB-CD2z, and m971-BBz-2A-CD19-28tm-CD2, from left to right. No cytokine release was observed in the absence of target tumor cells, and no measurable IL-2 was released by m971-BB-CD2z in the absence of CD58.

FIG. 24 shows that CAR-T cells, in which the CAR and the chimeric polypeptide target different epitopes of the same antigen, all perform better in cytotoxicity than CAR-T cells lacking the chimeric polypeptide, as described in Example 11.

FIG. 25 shows that trans construct m971-BBz-2A-HA22-28tm-CD2 performed better in cytokine release its cis analog, m971-BB-CD2z, as described in Example 11. In each case, the CAR constructs are m971-BB-CD2z and m971-BBz-2A-HA22-28tm-CD2, from left to right.

FIG. 26 shows that trans CD2 CART cells containing m971-BBz CAR expressed alongside a CD2 signaling CAR recognizing CD19 (CD22-4-1BBz+CD19-CD2) demonstrated strong anti-tumor activity against CD58KO Nalm6 compared to the traditional m971-BBz CAR expressed alongside a control molecule recognizing CD19 without any signaling domains (CD22-4-1BBz+CD19-TM).

FIG. 27 shows that trans CD22 CAR T cells co-expressed with CD2 containing receptor recognizing CD20 (m971-BBz+CD20-28tm-CD2 and m971-BBz+CD20-8tm-CD2) were able to rescue CAR T function against CD58 KO cells.

FIG. 28 shows that trans CD19 CAR T cells co-expressed with CD2 containing receptor recognizing CD20 (CD19-BBz+CD20-28htm-CD2) was able to rescue CAR T cell function against CD58 KO cells.

FIG. 29 shows that trans CD19-28z and m971-8tm-CD2-z was able to rescue CAR T function against CD58 KO cells and maintained activity against cells that lost either CD19 or CD22 antigen.

FIG. 30 shows that trans CD19-BBz and CD20-28htm-CD2-z was able to rescue CAR T cell function against CD58 KO cells and maintained activity against cells that lost the target antigen such as CD19.

FIG. 31 shows that CD19 or CD22 CAR cells integrating CD2 signaling in trans overcame CD58 loss and maintained activity against the cells that lost the target antigen CD19.

FIG. 32 shows that a CD20 targeted CAR that integrated CD2 signaling was able to overcome both loss of CD58 and loss of CD19, which are both common mechanisms of immune escape from CAR T cell therapy.

### DETAILED DESCRIPTION OF THE DISCLOSURE

CD58 (LFA-3) is a cell adhesion protein found primarily on antigen-presenting cells, such as lymphoblastoid cells (F. Sanchez-Madrid et al., Proc Natl Acad Sci USA (1982) 79:7489-93), B cells, T cells, monocytes, granulocytes, platelets, vascular endothelium, vascular smooth muscle, erythrocytes, and fibroblasts (A. M. Krensky et al., J Immunol (1983) 131(2):611-16). CD58 loss or mutation has been suggested as an unfavorable prognostic factor in DLBCL (T. Menter et al., Front Oncol (2018) 8:54).

CD2 (LFA-2) is a cell adhesion protein which is found on T cells and natural killer cells. Upon ligation by CD58, CD2 acts as a co-stimulator in T cells (P. Selvaraj et al., Nature (1987) 326:400-03; T. A. Springer, Nature (1990) 346:425-34), increasing cytokine production (B. E. Bierer et al., J Exp Med (1988) 168:1145-56) and reversing anergy (V. A. Boussiotis et al., J Exp Med (1994) 180:1665-73).

As set forth herein, the present disclosure shows that when CAR-T therapy fails in the treatment of lymphomas such as DLBCL and other B cell hyperproliferative disorders, one reason can be the loss, reduction, or mutation of CD58 on tumor cells. Unexpectedly, current CAR-T cells used in such therapy rely in part on tumor cell CD58 expression to activate endogenous CD2 present on patient-derived CAR-T cells. In the absence of CD58-CD2 signaling, CAR-T cells produce reduced quantities of IL-2 and IFNγ, resulting in concomitant reduction in activity and efficacy.

The present disclosure relates generally to methods for reducing or eliminating CAR-T dependence on tumor-expressed CD58; CARs that effectively incorporate CD2 signaling; co-stimulation of a CAR by a trans-CD2 signaling chimeric protein; and systems therefor.

### A. Definitions

The singular form “a”, “an”, and “the” include plural references unless the context clearly dictates otherwise. For example, the term “a cell” includes one or more cells, including mixtures thereof “A and/or B” is used herein to include all of the following alternatives: “A”, “B”, “A or B”, and “A and B.”

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

“Percent (%) amino acid sequence identity” or “homology” with respect to the nucleic acid or amino acid sequences identified herein is defined as the percentage of nucleic acid or amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved using publicly available sequence comparison computer program ALIGN-2. The source code for the ALIGN-2 sequence comparison computer program is available with user documentation in the U.S. Copyright Office, Washington D.C., 20559, where it is registered under U.S. Copyright Registration No. TXU510087. The ALIGN-2 program can be compiled for use on a UNIX operating system, such as a digital UNIX V4.0D. All sequence comparison parameters are set by the ALIGN-2 program and do not vary.

“Percent (%) identity” with respect to the nucleic acid or amino acid sequences identified herein is defined as the percentage of nucleic acid or amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved using publicly available sequence comparison computer program ALIGN-2. The source code for the ALIGN-2 sequence comparison computer program is available with user documentation in the U.S. Copyright Office, Washington D.C., 20559, where it is registered under U.S. Copyright Registration No. TXU510087. The ALIGN-2 program can be compiled for use on a UNIX operating system, such as a digital UNIX V4.0D. All sequence comparison parameters are set by the ALIGN-2 program and do not vary.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

As used herein, a “therapeutically effective amount” or “therapeutically effective number” of an agent is an amount or number sufficient to provide a therapeutic benefit in the treatment or management of a disease or disorder, or to delay or minimize one or more symptoms associated with the disease or disorder. A therapeutically effective amount of an agent means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the disease or disorder, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

Hyperproliferative disorders include cancers and hyperplasia characterized by the unregulated overgrowth of cells. Hyperproliferative disorders frequently display loss of genetic regulatory mechanisms, and may express native proteins inappropriately (including expression of proteins from other cell types or developmental stages, expression of mutated proteins, and expression of proteins at levels higher or lower than normal). CD58-hyperproliferative disorders are hyperproliferative disorders in which normal CD58 expression is reduced or absent, or in which CD58 is expressed in a mutated form.

B-cell hyperproliferative disorders include B-cell leukemias and lymphomas such as acute lymphoblastic leukemia (ALL), chronic lymphocytic leukemia (CLL), B-cell prolymphocytic leukemia, precursor B lymphoblastic leukemia, hairy cell leukemia, diffuse large B-cell lymphoma (DLBCL), follicular lymphoma, marginal zone lymphoma, mantle cell lymphoma, Burkitt's lymphoma, MALT lymphoma, Waldenstrom's macroglobulinemia, and other disorders characterized by the overgrowth of B-lineage cells.

An immune receptor, such as chimeric antigen receptors (CARs) and T-cell receptors (TCRs) in general comprise an extracellular antigen binding domain, a transmembrane domain, and a cytoplasmic signaling domain that activates the T cell cytotoxic response. The CAR frequently also has a spacer domain between the antigen binding domain and the transmembrane domain, which often includes a hinge domain. The cytoplasmic signaling domain can further comprise one or more co-stimulatory regions, as described herein.

CAR structures are often abbreviated to list the target antigen (or the antigen binding domain or agent); optionally the spacer domain; optionally the transmembrane domain; and the co-stimulatory and stimulatory domains of the cytoplasmic signaling domain. For example, a CAR having an anti-CD19 scFv antigen binding domain, a CD8a transmembrane domain (which may include the extracellular hinge region), a 4-1BB co-stimulatory domain, and a CD3ζ activating domain can be indicated as CD19-8tm-41BBz. Where the domains are commonly used they are often abbreviated even further, such that a CAR having a CD28 transmembrane domain, a 4-1BB domain, and a CD3ζ activating domain could be abbreviated as CD19-28tm-28BBz. These are often further abbreviated by omitting designation of the transmembrane domain, e.g., CD19-28BBz. It is also common to indicate a particular antigen binding domain rather than only its specificity, e.g., m971 rather than CD22, indicating that the antigen binding domain is the m971 scFv.

### B. CD2 CARs

CD2 CARs of the disclosure comprise an antigen binding domain, a spacer domain, a transmembrane domain, and a cytoplasmic signaling domain. The cytoplasmic signaling domain comprises a CD2 signaling (co-stimulating) domain, a second co-stimulating domain (other than CD28), and an activating domain such as a CD3ζ activating domain. CD2 CARs of the disclosure can be used in CAR-T cells either alone or in combination with other CARs. In some embodiments, CD2 CARs of the present disclosure can be used in combination with a chimeric polypeptide and/or a transgenic T-cell receptor (TCR). In some embodiments, CD2 signaling is enhanced by the transgenic TCR. In some embodiments, CD2 CARs of the present disclosure can be used in combination with one or more additional therapeutic agents such as, for example, an antibody or an antigen binding fragment thereof, or a molecule (administered, secreted, or surface expressed) that can crosslink native CD2 in response to a tumor specific antigen in the tumor microenvironment. As set forth herein, the CD2 CARs of the disclosure render a CAR-T cell less dependent on CD58 expression by target cells, and can further increase the therapeutic activity of a CAR-T cell.

In some embodiments, the CAR is a first generation CAR. In some embodiments, the CAR is a second generation CAR. In some embodiments, the CAR is a third generation CAR. A first generation CAR generally has an intracellular signaling domain comprising an intracellular signaling domain of CD3z, FcyRI, or other ITAM-containing activating domain to provide a T cell activation signal. Second generation CARs further comprise a costimulatory signaling domain (e.g., a costimulatory signaling domain from an endogenous T cell costimulatory receptor, such as CD28, 4-1BB, or ICOS). Third generation CARs may comprise an EGAM-containing activating domain, a first costimulatory signaling domain and a second costimulatory signaling domain.

1. Antigen Binding Domain

The antigen binding domain can be any molecule that binds to the selected antigen with sufficient affinity and specificity, and is often an antibody or an antibody derivative, such as an scFv, single domain antibody (sdAb), Fab′ fragment, (Fab′)₂ fragment, nanobody, diabody, or the like. Alternatively, the antigen binding domain can be a receptor or a receptor fragment that binds specifically to the target antigen. The antigen binding domain can be attached to the rest of the receptor directly (covalently) or indirectly (for example, through the noncovalent binding of two or more binding partners), as described below.

The first antigen binding domain is selected for specific binding to a target antigen. In general, the target antigen is selected because it is characteristic of a target cell, such as a tumor cell, and not characteristic of other (healthy) cells. Tumor antigens are proteins that are produced by tumor cells that elicit an immune response, particularly T-cell mediated immune responses. Thus, an antigen binding moiety can be selected based on the particular type of cancer to be treated. Tumor antigens include, for example without limitation, glioma-associated antigen, carcinoembryonic antigen (CEA), beta-human chorionic gonadotropin, alpha-fetoprotein (AFP), lectin-reactive AFP, thyroglobulin, RAGE-1, MN-CA IX, human telomerase reverse transcriptase, RU1, RU2 (AS), intestinal carboxyl esterase, mut HSP70-2, M-CSF, prostate-specific antigen (PSA), PAP, NY-ESO-1, LAGE-1a, p53, prostein, PSMA, HER2, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, GD2, GD3, B7-H3, GPC2, L1CAM, EGFR, and mesothelin.

The tumor antigen may also be a tumor-specific antigen (TSA) or a tumor-associated antigen (TAA). A TSA is unique to tumor cells, and does not occur on other cells in the body. A TAA is not unique to a tumor cell, and is also expressed on some normal cells under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond, or may be antigens that are normally present at low levels on normal cells but which are expressed at much higher levels on tumor cells.

Examples of TSA and TAA include, without limitation, differentiation antigens such as MART-1/MelanA (MART-1), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO-1, p185erbB2, p180erbB-3, c-met, nm-23H1, PSA, TAG-72, CA 19-9, CA 72-4, CAM 17.1, NuMa, K-ras, beta-Catenin, CDK4, Mum-1, p15, p16, 43-9F, 5T4, 791Tgp72, alpha-fetoprotein, beta-HCG, BCA225, BTAA, CA125, BCAA, CA195, CA242, CA-50, CAM43, CD68/P1, CO-029, FGF-5, G250, Ga733/EpCAM, HTgp-175, M344, MA-50, MG7-Ag, MOV18, NB/70K, NY-CO-1, RCAS1, SDCCAG16, TA-90, TAAL6, TAG72, TLP, and TPS. In some embodiments, the target antigen is CD19, CD20, CD22, ROR1, or GD2. In some embodiments, the target antigen is CD19, CD20, or CD22. In an embodiment, the target antigen is CD19. In an embodiment, the target antigen is CD22.

Antibody derivatives are molecules that resemble antibodies in their mechanism of ligand binding, and include, for example, nanobodies, duobodies, diabodies, triabodies, minibodies, F(ab′)₂ fragments, Fab fragments, single chain variable fragments (scFv), single domain antibodies (sdAb), and functional fragments thereof. See for example, D. L. Porter et al., N Engl J Med (2011) 365(8):725-33 (scFv); E. L. Smith et al., Mol Ther (2018) 26(6):1447-56 (scFv); S. R. Banihashemi et al., Iran J Basic Med Sci (2018) 21(5):455-64 (CD19 nanobody); F. Rahbarizadeh et al., Adv Drug Deliv Rev (2019) 141:41-46 (sdAb); S. M. Kipriyanov et al., Int J Cancer (1998) 77(5):763-72 (diabody); F. Le Gall et al., FEBS Lett (1999) 453(1-2):164-68 (triabody); M. A. Ghetie et al., Blood (1994) 83(5):1329-36 (F(ab′)₂); and M. A. Ghetie et al., Clin Cancer Res (1999) 5(12):3920-27 (F(ab′)₂ and Fab′). Antibody derivatives can also be prepared from therapeutic antibodies, for example without limitation, by preparing a nanobody, duobody, diabody, triabody, minibody, F(ab′)₂ fragment, Fab fragment, single chain variable fragment (scFv), or single domain antibody (sdAb) based on a therapeutic antibody. Antibody derivatives can also be designed using phage display techniques (see, e.g., E. Romao et al., Curr Pharm Des (2016) 22(43):6500-18).

The antigen binding domain may include binding domains for multiple antigens, which may be the same or different. For example, the antigen binding domain can comprise a bispecific (Fab′)₂, specific for two antigens, or for two epitopes on the same antigen. Multispecific antigen binding domains can increase the sensitivity of the CAR, for example by allowing the CAR to recognize and react to multiple antigens.

The antigen binding domain can alternatively be expressed independently from the rest of the CAR, and bind to it through non-covalent interactions. For example, the extracellular portion of the CAR can comprise one member of a specific binding pair, which binds to the independent antigen binding domain (without interfering with antigen binding by the antigen binding domain). For example, the CAR extracellular domain can comprise streptavidin, while the independent antigen binding domain is biotinylated. Alternatively, the CAR extracellular domain can comprise an antibody or antibody derivative that is specific for the independent antigen binding domain. This division into independent antigen binding domain and CAR enables one to change the antigen specificity of the receptor without transducing a new receptor. See, e.g., N. G. Minutolo et al, Front Oncol (2019) 9:176.

2. Transmembrane Domain

The transmembrane domain serves to link the extracellular domain (antigen binding domain and spacer domain) of the receptor with the cytoplasmic domain. In general, any transmembrane domain capable of working in a CAR can be used in the receptors and methods of the disclosure. The transmembrane domain may include, for example without limitation, all or part of the transmembrane domain of the CD3 zeta chain (CD3ζ), CD28, CD2, CD4, OX40, 4-1BB (CD137), FcERIγ, ICOS (CD278), ILRB (CD122), IL-2RG (CD132), CTLA-4, PD-1, or CD40, or a sequence derived from such a transmembrane domain. The cytoplasmic signaling domain in general comprises a domain that transduces the event of ligand binding into an intracellular signal that activates the T cell. The CD3ζ intracellular domain/activating domain is frequently used, although others such as MyD88 can be used. In an embodiment, the transmembrane domain is the transmembrane domain from CD3ζ, CD2, CD8, or CD28. In an embodiment, the transmembrane domain is derived from the transmembrane domain from CD2 or CD28. In some embodiments, the transmembrane domain has about 70, 75, 80, 85, 90, 92, 93, 94, 95, 96, 97, 98, 99 or about 100% sequence identity to a CD3ζ, CD28, CD2, CD4, OX40, 4-1BB (CD137), FcERIγ, ICOS (CD278), ILRB (CD122), IL-2RG (CD132), or CD40 transmembrane domain.

3. Spacer Domain

In some embodiments, the CAR further includes an extracellular spacer domain, which may include a hinge domain. The hinge domain is generally a flexible polypeptide connector region disposed between the targeting moiety and the transmembrane domain. Hinge domain sequences are often derived from IgG subclasses (such as IgG1 and IgG4), IgD, CD28, and CD8 domains. In some embodiments, the hinge domain provides structural flexibility to flanking polypeptide regions. The hinge domain may consist of natural or synthetic polypeptides. It will be appreciated by those skilled in the art that hinge domains may improve the function of the CAR by promoting optimal positioning of the antigen binding moiety in relationship to the portion of the antigen recognized by it. In some embodiments, a hinge domain may not be required for optimal CAR activity. In some embodiments, a hinge domain comprising a short sequence of amino acids promotes CAR activity by facilitating antigen-binding by, for example, relieving steric constraints that could otherwise alter antibody binding kinetics. In some embodiments, the hinge domain is linked downstream of the antigen-binding moiety and upstream of the transmembrane domain.

Non-limiting examples of suitable hinge domains include those derived from CD8a, CD28, CTLA4, CD4, PD1, IgG1, PGK, or IgG4. In some embodiments, the hinge domain can include regions derived from a human CD8α (also known as CD8a) molecule, a CD28 molecule, and any other receptors that provide a similar function in providing flexibility to flanking regions. In some embodiments, the CAR disclosed herein includes a hinge domain derived from a CD8α hinge domain. In some embodiments, the CAR disclosed herein includes a hinge domain derived from a CD28 or CD2 hinge domain. In some embodiments, the hinge domain has about 70, 75, 80, 85, 90, 92, 93, 94, 95, 96, 97, 98, 99 or about 100% sequence identity to a CD8α, CD28, CTLA4, CD4, PD1, IgG1, PGK, or IgG4 hinge domain.

In some embodiments, the spacer domain further comprises a linker including one or more intervening amino acid residues that are positioned between the antigen binding domain and the extracellular hinge domain. In some embodiments, the linker is positioned downstream from the antigen binding domain and upstream from the hinge domain. In principle, there are no particular limitations to the length and/or amino acid composition of the linker. In some embodiments, any arbitrary single-chain peptide comprising about one to about 300 amino acid residues (e.g., 2, 3, 4, 5, 6, 7, 8, 9, 10, 11, 12, 13, 14, 15, 16, 17, 18, 19, 20, or more amino acid residues) can be used as a linker. In some embodiments, the linker includes at least about 5, 6, 7, 8, 9, 10, 11, 12, 15, 20, 25, 30, 35, 40, 45, or 50 amino acids. In some embodiments, the linker includes no more than about 300, 250, 200, 150, 140, 130, 120, 110, 100, 90, 85, 80, 75, 70, 65, 60, 55, 50, 45, 40, 35, or 30 amino acid residues. In some embodiments, the length and amino acid composition of the extracellular spacer can be optimized to vary the orientation and/or proximity of the antigen binding domain and the extracellular hinge domain to one another to achieve a desired activity of the CAR. In some embodiments, the orientation and/or proximity of the antigen binding domain and the extracellular hinge domain to one another can be varied and/or optimized as a “tuning” tool or effect to enhance or reduce the efficacy of the CAR. In some embodiments, the orientation and/or proximity of the antigen binding domain and the hinge domain to one another can be varied and/or optimized to create a partially functional version of the CAR. In some embodiments, the extracellular spacer domain includes an amino acid sequence corresponding to an IgG4 hinge domain and an IgG4 CH2-CH3 domain.

Alternatively, the spacer domain can be a synthetic polypeptide spacer, such as a spacer having a random sequence, a (gly-gly-ser)ₙ (“GGSn”) sequence, or a variation thereof such as (SGG)ₙ, (GGGS)ₙ, (SGGG)ₙ, (GSGGG)ₙ, and the like, where n can range from about 1 to about 15 (SEQ ID NOs 87-91). The synthetic polypeptide spacer domain can also include a naturally-occurring sequence, such as a hinge domain derived from CD8α, IgG, and the like.

4. Cytoplasmic Signaling Domain

The cytoplasmic signaling domain, in general, comprises an activating domain having an immunoreceptor tyrosine-based activation motif (ITAM), which when phosphorylated activates the T cell reaction to an antigen. Phosphorylation occurs as a result of antigen binding. The activating domain is most often derived from CD3ζ.

CARs of the disclosure comprise a CD3ζ activating domain, a CD2 co-stimulatory domain, and one or more additional co-stimulatory domains to increase cytokine production or sensitivity, reduce or prevent anergy, and/or to increase proliferation and cytotoxic activity. These additional co-stimulatory domains can be derived from co-stimulatory proteins such as B7-1 (CD80), B7-2 (CD86), CTLA-4, PD-1, CD278, CD122, CD132, B7-H2, B7-H3, PD-L1, PD-L2, B7-H4, PDCD6, BTLA, 41BB (CD137), FcERIγ, CD40L, 4-1BBL, GITR, BAFF, GITR-L, BAFF-R, HVEM, CD27, LIGHT, CD27L, OX40, OX40L, CD30, CD30L, TAC1, CD40, CD244, CD84, BLAME, CD229, CRACC, CD2F-10, NTB-A, CD48, SLAM (CD150), CD58, ikaros, CD53, integrin α4, CD82, integrin α4β1, CD90, integrin α4β7, CD96, LAG-3, CD160, LMIR, CRTAM, TCL1A, DAP12; TIM-1, Dectin-1, TIM-4, TSLP, EphB6, TSLP-R, and HLA-DR. In an embodiment of the disclosure, the cytoplasmic signaling domain comprises a CD3ζ activating domain. In some embodiments, one co-stimulatory domain is a CD2 co-stimulatory domain. In another embodiment, the cytoplasmic signaling domain comprises a CD2 co-stimulatory domain and a second co-stimulatory domain, wherein the second co-stimulatory domain does not comprise a CD28 co-stimulatory domain. In an embodiment, the second co-stimulatory domain comprises a 41BB co-stimulatory domain. CD28 co-stimulatory domains are not effective in the practice of the methods described herein, and fail to provide activation in the absence of CD58. In some embodiments, the co-stimulating domain comprises at least one of: a 4-1BB signaling domain, a CD27 signaling domain, an OX40 signaling domain, a CD28 signaling domain, a CD278 signaling domain, a CD40 signaling domain, a CD40L signaling domain, a toll-like receptor signaling domain, or any combination thereof (Weinkove R. et al., Clin Transl Immunology. 2019; 8(5):e1049).

5. Exemplary Constructs

Exemplary CARs of the present disclosure can include, but not limited to, any one of the following: CD19-CD2-z (SEQ ID NOs: 12, 41), CD19-CD2-BBz (SEQ ID NOs: 13, 42), CD19-BB-CD2z (SEQ ID NOs: 14, 43), CD19-CD2-28z (SEQ ID NOs: 15, 44), m971-CD2z (SEQ ID NOs: 16, 45), m971-CD2-BBz (SEQ ID NOs: 17, 46), m971-CD2-BB-CD2z (SEQ ID NOs: 18, 47), m971-CD2-28z (SEQ ID NO: 19, 48), CD19-28tm-CD2 (SEQ ID NOs: 20, 49), CD19-8tm-CD2 (SEQ ID NOs: 21, 50), CD19-2tm-CD2 (SEQ ID NOs: 22, 51), m971-28tm-CD2 (SEQ ID NOs: 23, 52), m971-8tm-CD2 (SEQ ID NOs: 24, 53), HA22-28tm-CD2 (SEQ ID NOs: 25, 54), HA22-8tm-CD2 (SEQ ID NOs: 26, 55), HA22-2tm-CD2 (SEQ ID NOs: 27, 56), CD20-28tm-CD2 (SEQ ID NOs: 28, 57), CD20-8tm-CD2 (SEQ ID NOs: 29, 58), m971-BBz (SEQ ID NOs: 59, 60), m971-28z (SEQ ID NOs: 61, 62), CD19-28z (SEQ ID NOs: 63, 64), CD19-BBz (SEQ ID NOs: 65, 66), m971-BBz-CD2 (SEQ ID NOs: 67, 68), m971-BBz-2A-CD19-CD28tm-CD2 (SEQ ID NOs: 71, 72), m971-BBz-2A-CD19-CD8tm-CD2 (SEQ ID NOs: 73, 74), m971-BBz-2A-CD19-CD2tm-CD2 (SEQ ID NOs: 75, 76), m971-BBz-2A-HA22-CD28tm-CD2 (SEQ ID NOs: 81, 82), m971-BBz-2A-HA22-CD8tm-CD2 (SEQ ID NOs: 83, 84), m971-BBz-2A-HA22-2tm-CD2 (SEQ ID NOs: 85, 86), CD19-8htm-CD2-z (SEQ ID NOs: 92, 114), CD19-8htm-CD2-BB-z (SEQ ID NOs: 93, 115), CD19-8htm-BB-CD2-z (SEQ ID NOs: 94, 116), CD19-8htm-CD2-28-z (SEQ ID NOs: 95, 117), CD19-28htm-CD2-z (SEQ ID NOs: 96, 118), m971-8htm-CD2-z (SEQ ID NOs: 97, 119), m971-8htm-CD2-BB-z (SEQ ID NOs: 98, 120), m971-8htm-BB-CD2-z (SEQ ID NOs: 99, 121), m971-8htm-CD2-28-z (SEQ ID NOs: 100, 122), CD19-28htm-CD2 (SEQ ID NOs: 101, 123), CD19-8htm-CD2 (SEQ ID NOs: 102, 124), CD19-2htm-CD2 (SEQ ID NOs: 103, 125), m971-28htm-CD2 (SEQ ID NOs: 104, 126), m971-8htm-CD2 (SEQ ID NOs: 105, 127), HA22-28htm-CD2 (SEQ ID NOs: 106, 128), HA22-8htm-CD2 (SEQ ID NOs: 107, 129), HA22-2htm-CD2 (SEQ ID NOs: 108, 130), CD20-IgG1long-28htm-CD2 (SEQ ID NOs: 109, 131), CD20-IgG1long-8htm-CD2 (SEQ ID NOs: 110, 132), CD20-IgG1long-28htm-CD2-z (SEQ ID NOs: 111, 133), or CD20-IgG1long-8htm-CD2-z (SEQ ID NOs: 112, 134).

In some embodiments, the CAR of the present disclosure can comprise an amino acid sequence or a nucleotide sequence that is at least about 70%, about 80%, about 90%, about 91%, about 92%, about 93%, about 94%, about 95%, about 96%, about 97%, about 98%, about 99%, or about 100% identical to a sequence selected from the group consisting of: CD19-CD2-z (SEQ ID NOs: 12, 41), CD19-CD2-BBz (SEQ ID NOs: 13, 42), CD19-BB-CD2z (SEQ ID NOs: 14, 43), CD19-CD2-28z (SEQ ID NOs: 15, 44), m971-CD2z (SEQ ID NOs: 16, 45), m971-CD2-BBz (SEQ ID NOs: 17, 46), m971-CD2-BB-CD2z (SEQ ID NOs: 18, 47), m971-CD2-28z (SEQ ID NO: 19, 48), CD19-28tm-CD2 (SEQ ID NOs: 20, 49), CD19-8tm-CD2 (SEQ ID NOs: 21, 50), CD19-2tm-CD2 (SEQ ID NOs: 22, 51), m971-28tm-CD2 (SEQ ID NOs: 23, 52), m971-8tm-CD2 (SEQ ID NOs: 24, 53), HA22-28tm-CD2 (SEQ ID NOs: 25, 54), HA22-8tm-CD2 (SEQ ID NOs: 26, 55), HA22-2tm-CD2 (SEQ ID NOs: 27, 56), CD20-28tm-CD2 (SEQ ID NOs: 28, 57), CD20-8tm-CD2 (SEQ ID NOs: 29, 58), m971-BBz (SEQ ID NOs: 59, 60), m971-28z (SEQ ID NOs: 61, 62), CD19-28z (SEQ ID NOs: 63, 64), CD19-BBz (SEQ ID NOs: 65, 66), m971-BBz-CD2 (SEQ ID NOs: 67, 68), m971-BBz-2A-CD19-CD28tm-CD2 (SEQ ID NOs: 71, 72), m971-BBz-2A-CD19-CD8tm-CD2 (SEQ ID NOs: 73, 74), m971-BBz-2A-CD19-CD2tm-CD2 (SEQ ID NOs: 75, 76), m971-BBz-2A-HA22-CD28tm-CD2 (SEQ ID NOs: 81, 82), m971-BBz-2A-HA22-CD8tm-CD2 (SEQ ID NOs: 83, 84), m971-BBz-2A-HA22-2tm-CD2 (SEQ ID NOs: 85, 86), CD19-8htm-CD2-z (SEQ ID NOs: 92, 114), CD19-8htm-CD2-BB-z (SEQ ID NOs: 93, 115), CD19-8htm-BB-CD2-z (SEQ ID NOs: 94, 116), CD19-8htm-CD2-28-z (SEQ ID NOs: 95, 117), CD19-28htm-CD2-z (SEQ ID NOs: 96, 118), m971-8htm-CD2-z (SEQ ID NOs: 97, 119), m971-8htm-CD2-BB-z (SEQ ID NOs: 98, 120), m971-8htm-BB-CD2-z (SEQ ID NOs: 99, 121), m971-8htm-CD2-28-z (SEQ ID NOs: 100, 122), CD19-28htm-CD2 (SEQ ID NOs: 101, 123), CD19-8htm-CD2 (SEQ ID NOs: 102, 124), CD19-2htm-CD2 (SEQ ID NOs: 103, 125), m971-28htm-CD2 (SEQ ID NOs: 104, 126), m971-8htm-CD2 (SEQ ID NOs: 105, 127), HA22-28htm-CD2 (SEQ ID NOs: 106, 128), HA22-8htm-CD2 (SEQ ID NOs: 107, 129), HA22-2htm-CD2 (SEQ ID NOs: 108, 130), CD20-IgG1long-28htm-CD2 (SEQ ID NOs: 109, 131), CD20-IgG1long-8htm-CD2 (SEQ ID NOs: 110, 132), CD20-IgG1long-28htm-CD2-z (SEQ ID NOs: 111, 133), or CD20-IgG1long-8htm-CD2-z (SEQ ID NOs: 112, 134).

### C. Chimeric Polypeptides

An aspect of the disclosure is a chimeric polypeptide for use with a CAR in a CAR-T therapy and/or in combination with a transgenic TCR. Chimeric polypeptides of the disclosure comprise, from N-terminal to C-terminal, an antigen binding domain, an optional spacer domain, a transmembrane domain, and a cytoplasmic signaling domain, wherein the cytoplasmic signaling domain comprises a CD2 signaling (co-stimulating) domain, and does not comprise a CD3ζ activating domain. These chimeric polypeptides function as a trans-CD2 signaling receptor, and differ from CARs in that they lack an activating domain, such as a CD3ζ activating domain or equivalent. Thus, they do not directly activate T cells, but act as a co-receptor to the CAR.

The antigen binding domain can be selected from the set of antigen binding domains described above, and the target antigens described above, or any other suitable target. Suitable targets will be antigens that are found on the target cells, and need not be tumor-specific or tumor-associated as the chimeric polypeptide does not trigger immune cell activation in the absence of CAR or T cell receptor activation. However, using a tumor-specific or tumor-associated as the chimeric polypeptide target can increase specificity of the engineered cell for the target cells, particularly where the CAR and the chimeric polypeptide have different target antigens.

The CAR and the chimeric polypeptide antigen binding domains (first antigen binding domain and second antigen binding domain, respectively) can target the same antigen, different antigens, or different epitopes of the same antigen. In an embodiment, the first antigen binding domain and the second antigen binding domain target different antigens. In an embodiment, the first antigen binding domain and the second antigen binding domain target different antigens. In an embodiment, the first antigen binding domain and the second antigen binding domain target different epitopes of the same antigen. In an embodiment, the first antigen binding domain is specific for CD19 or CD22. In an embodiment, the second antigen binding domain is specific for CD22 or CD19.

As described above, the antigen binding domain of the chimeric polypeptide can be multispecific, and the chimeric polypeptide and the CAR can target multiple different or partially overlapping antigens.

The chimeric polypeptide transmembrane domain may include any of the transmembrane domains described above, including without limitation, all or part of the transmembrane domain of the CD3 zeta chain (CD3ζ), CD2, CD28, OX40, 4-1BB (CD137), FcERIγ, ICOS (CD278), ILRB (CD122), IL-2RG (CD132), CTLA-4, PD-1, or CD40. It is sometimes observed that having two different receptors with identical transmembrane domains results in interference or reduction of activity. Accordingly, different transmembrane domains for the CAR and the chimeric polypeptide can be selected. In an embodiment, the transmembrane domain is the transmembrane domain from CD3ζ, CD2, CD8, or CD28. In an embodiment, the transmembrane domain is the transmembrane domain from CD2 or CD28. In an embodiment, the transmembrane domain is the transmembrane domain from CD28. In some embodiments, the transmembrane domain of the CAR and the transmembrane domain of the chimeric polypeptide are different.

The chimeric polypeptide can further comprise a spacer domain and/or hinge domain, as described above. In an embodiment, the chimeric polypeptide comprises a CD8α hinge domain or a CD28 hinge domain or a CD2 hinge domain. In an embodiment, the chimeric polypeptide comprises a CD28 hinge domain.

The chimeric polypeptide cytoplasmic signaling domain does not comprise a CD3ζ activating domain, but does comprise at least a CD2 signaling domain. The cytoplasmic signaling domain can further comprise an additional co-stimulating signaling domain, other than a CD28 signaling domain. In an embodiment, the chimeric polypeptide cytoplasmic signaling domain comprises CD2. In an embodiment, the chimeric polypeptide cytoplasmic signaling domain comprises CD2 and 4-1BB. In an embodiment, the chimeric polypeptide cytoplasmic signaling domain comprises CD2 and OX40. In some embodiments, the co-stimulating domain comprises at least one of: a 4-1BB signaling domain, a CD27 signaling domain, an OX40 signaling domain, a CD28 signaling domain, a CD278 signaling domain, a CD40 signaling domain, a CD40L signaling domain, a toll-like receptor signaling domain, or any combination thereof (Weinkove R. et al., Clin Transl Immunology. 2019; 8(5):e1049).

In some embodiments, the antigen binding domain comprises an amino acid sequence that is at least about 70%, about 80%, about 90%, about 91%, about 92%, about 93%, about 94%, about 95%, about 96%, about 97%, about 98%, about 99%, or about 100% homologous to a sequence selected from the group consisting of SEQ ID NOs: 1, 2, 3, and 4. In some embodiments, the antigen binding domain comprises an amino acid sequence that is at least about 70%, about 80%, about 90%, about 91%, about 92%, about 93%, about 94%, about 95%, about 96%, about 97%, about 98%, about 99%, or about 100% identical to a sequence selected from the group consisting of SEQ ID NOs: 1, 2, 3, and 4.

In some embodiments, the transmembrane domain comprises an amino acid sequence that is at least about 70%, about 80%, about 90%, about 91%, about 92%, about 93%, about 94%, about 95%, about 96%, about 97%, about 98%, about 99%, or about 100% homologous to a sequence selected from the group consisting of SEQ ID NOs: 5, 6, and 7. In some embodiments, the transmembrane domain comprises an amino acid sequence that is at least about 70%, about 80%, about 90%, about 91%, about 92%, about 93%, about 94%, about 95%, about 96%, about 97%, about 98%, about 99%, or about 100% identical to a sequence selected from the group consisting of SEQ ID NOs: 5, 6, and 7.

In some embodiments, the CD2 signaling domain comprises an amino acid sequence that is at least about 70%, about 80%, about 90%, about 91%, about 92%, about 93%, about 94%, about 95%, about 96%, about 97%, about 98%, about 99%, or about 100% homologous to the sequence of SEQ ID NO: 8. In some embodiments, the CD2 signaling domain comprises an amino acid sequence that is at least about 70%, about 80%, about 90%, about 91%, about 92%, about 93%, about 94%, about 95%, about 96%, about 97%, about 98%, about 99%, or about 100% identical to the sequence of SEQ ID NO: 8.

In some embodiments, the co-stimulating signaling domain comprises an amino acid sequence that is at least about 70%, about 80%, about 90%, about 91%, about 92%, about 93%, about 94%, about 95%, about 96%, about 97%, about 98%, about 99%, or about 100% homologous to a sequence selected from the group consisting of SEQ ID NOs: 10 and 11. In some embodiments, the co-stimulating signaling comprises an amino acid sequence that is at least about 70%, about 80%, about 90%, about 91%, about 92%, about 93%, about 94%, about 95%, about 96%, about 97%, about 98%, about 99%, or about 100% identical to a sequence selected from the group consisting of SEQ ID NOs: 10 and 11.

In some embodiments, the activating domain comprises an amino acid sequence that is at least about 70%, about 80%, about 90%, about 91%, about 92%, about 93%, about 94%, about 95%, about 96%, about 97%, about 98%, about 99%, or about 100% homologous to the sequence of SEQ ID NO: 9. In some embodiments, the activating domain comprises an amino acid sequence that is at least about 70%, about 80%, about 90%, about 91%, about 92%, about 93%, about 94%, about 95%, about 96%, about 97%, about 98%, about 99%, or about 100% identical to the sequence of SEQ ID NO: 9.

### D. T CeH Receptors

An aspect of the disclosure is a transgenic T cell receptor for use with a CAR in a CAR-T therapy. A TCR can be found on the surface of a cell or in soluble form. Generally, a TCR is found on the surface of T cells (or T lymphocytes) where it is generally responsible for recognizing antigens bound to major histocompatibility complex (MHC) molecules.

T cell receptor (TCR) is a heterodimeric cell surface protein of the immunoglobulin superfamily that participate in the activation of T cells in response to the binding of an antigen. The TCR complex can consist of TCRα/β chains and CD3γ/δ/ε/ζ subunits, which can associate through hydrophobic interactions. Somatic VDJ recombination enables the generation of distinct TCRα and TCRβ chains, and TCRαβ heterodimers are generally responsible for antigen recognition by binding to peptide-MHC complexes. CD3 can transmits the TCR-triggered signal through immunoreceptor tyrosine-based activation motifs (ITAMs) in its cytoplasmic tail, but it is generally not directly involved in antigen recognition. ITAMs are tandem duplications of a tyrosine-containing sequence (YXXL/I), and the CD3γ/δ/ε chains each contain one ITAM, while the CD3ζ chain contains three. As a consequence of TCR engagement, ITAM phosphorylation can be induced by protein tyrosine kinases (PTKs), which allow other effector molecules to interact with the TCR complex.

In some embodiments, a TCR can be an intact or full-length TCR, including a TCR in the αβ form or γδ form. In some embodiments, a TCR is a dimeric TCR (dTCR). In some embodiments, a TCR is a single-chain TCR (scTCR). In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, a TCR can contain a constant domain, a transmembrane domain and/or a short cytoplasmic tail. In some embodiments, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR contains one or more constant domain. For example, the extracellular portion of a given TCR chain (e.g., α chain or β chain) can contain two immunoglobulin-like domains, such as a variable domain (e.g., Vα or Vβ) and a constant domain (e.g., Cα or Cβ) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR can contain short connecting sequences in which a cysteine residue forms a disulfide bond, thereby linking the two chains of the TCR. In some embodiments, a TCR can have an additional cysteine residue in each of the α and β chains, such that the TCR contains two disulfide bonds in the constant domains.

In some embodiments, a TCR is a dimeric TCR (dTCR). In some embodiments, the dTCR can contain a first polypeptide wherein a sequence corresponding to a TCRα chain variable region sequence is fused to the N terminus of a sequence corresponding to a TCRα chain constant region extracellular sequence, and a second polypeptide wherein a sequence corresponding to a TCRβ chain variable region sequence is fused to the N terminus a sequence corresponding to a TCRβ chain constant region extracellular sequence, the first and second polypeptides being linked by a disulfide bond. In some embodiments, the bond can correspond to the native interchain disulfide bond present in native dimeric TCRαβ forms. In some embodiments, the interchain disulfide bonds are not present in a native TCR. For example, in some embodiments, one or more cysteines can be incorporated into the constant region extracellular sequences of dTCR polypeptide pair. In some embodiments, a dTCR can have both a native and one or more non-native disulfide bonds. In some embodiments, a dTCR can contain a transmembrane sequence to anchor to the membrane. In some embodiments, a dTCR can contain a TCRα chain containing a variable α domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, a TCR is a single-chain TCR (scTCR). In some embodiments, a scTCR can contain a non-native disulfide interchain bond to facilitate the association of the TCR chains. In some embodiments, a scTCR is a non-disulfide linked truncated TCR in which heterologous leucine zippers fused to the C-termini thereof facilitating chain association. In some embodiments, a scTCR contain a TCRα variable domain covalently linked to a TCRβ variable domain via a linker. In some embodiments, the linker can be any linker capable of forming a single polypeptide strand, while retaining TCR binding specificity. In some embodiments, a scTCR can contain a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some embodiments, a scTCR can contain both a native and a non-native disulfide bond.

In some embodiments, a TCR or antigen binding portion thereof is one that has been modified or engineered. In some embodiments, the antigen binding domain of a TCR can be multispecific. In some embodiments, a TCR or antigen-binding portion thereof can be a recombinantly produced natural protein or a mutated form thereof in which one or more property, such as a binding characteristic, has been altered. In some embodiments, directed evolution methods are used to generate TCRs with altered properties, such as with higher affinity for a specific MHC-peptide complex. In some embodiments, directed evolution is achieved by display methods including, but not limited to, yeast display. In some embodiments, display approaches can involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, can be selected. In some embodiments, peptides suitable for use in generating TCRs or antigen-binding portions can be determined based on the presence of an HLA-restricted motif in a target polypeptide of interest. In some embodiments, peptides are identified using computer prediction models known to those of skill in the art.

In some embodiments, a TCR can be generated from a known TCR sequence(s), such as sequences of Vα and/or Vβ chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, a TCR is obtained from a biological source, such as from a T cell (e.g., cytotoxic T cell), T-cell hybridomas or other publicly available source. In some embodiments, the T-cells can be obtained from in vivo isolated cells, e.g., from a human subject. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof can be synthetically generated from knowledge of the sequence of the TCR.

In some embodiments, a TCR is generated from the TCR identified or selected from screening a library of candidate TCRs against a target polypeptide antigen, or target T cell epitope thereof. TCR libraries can be generated by amplification of the repertoire of Vu and VP from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ cells. In some embodiments, the TCRs can be amplified from a T cell source of a normal of healthy subject, i.e., normal TCR libraries. In some embodiments, the TCRs can be amplified from a T cell source of a diseased subject, i.e., diseased TCR libraries. In some embodiments, degenerate primers are used to amplify the gene repertoire of Vα and Vα, such as by RT-PCR in samples, such as T cells, obtained from humans. In some embodiments, scTv libraries can be assembled from naive Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some embodiments, particular residues within CDRs of the TCR can be altered. In some embodiments, selected TCRs can be modified by affinity maturation. In some embodiments, antigen-specific T cells can be selected, such as by screening to assess CTL activity against the peptide. In some aspects, TCRs, e.g., present on the antigen-specific T cells, may be selected, such as by binding activity, e.g., particular affinity or avidity for the antigen.

In some embodiments, a TCR of the present disclosure binds to a tumor specific antigen or tumor-associated antigen. In some embodiments, the tumor specific antigen or tumor-associated antigen is selected from the group consisting of glioma-associated antigen, carcinoembryonic antigen (CEA), beta-human chorionic gonadotropin, alpha-fetoprotein (AFP), lectin-reactive AFP, thyroglobulin, RAGE-1, MN-CA IX, human telomerase reverse transcriptase, RU1, RU2 (AS), intestinal carboxyl esterase, mut HSP70-2, M-CSF, prostate-specific antigen (PSA), PAP, NY-ESO-1, LAGE-1a, p53, prostein, PSMA, HER2, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, GD2, GD3, B7-H3, GPC2, L1CAM, EGFR, mesothelin, MART-1, gp100 (Pmel 17), tyrosinase, TRP-1, TRP-2, MAGE-1, MAGE-3, BAGE, GAGE-1, GAGE-2, p15, p53, Ras, HER-2, BCR-ABL, E2A-PRL, H4-RET, IGH-IGK, MYL-RAR, EBVA, human papillomavirus (HPV) antigens E6 and E7, TSP-180, MAGE-4, MAGE-5, MAGE-6, RAGE, NY-ESO-1, p185erbB2, p180erbB-3, c-met, nm-23H1, PSA, TAG-72, CA 19-9, CA 72-4, CAM 17.1, NuMa, K-ras, β-Catenin, CDK4, Mum-1, p15, p16, 43-9F, 5T4, 791Tgp72, α-fetoprotein, β-HCG, BCA225, BTAA, CA125, BCAA, CA195, CA242, CA-50, CAM43, CD68/P1, CO-029, FGF-5, G250, Ga733/EpCAM, HTgp-175, M344, MA-50, MG7-Ag, MOV18, NB/70K, NY-CO-1, RCAS1, SDCCAG16, TA-90, TAAL6, TAG72, TLP, TPS CD19, CD20, CD22, ROR1, and GD2.

In some embodiments, a TCR of the present disclosure can fuse the CD2 intracellular domain to the C-terminus of CD3z. In some embodiments, a CD3-epsilon can be produced in which the CD2 intracellular domain can be fused to the C terminus of CD3-epsilon or in which the intracellular portion of CD2 can replace all or part of the intracellular portion of CD3-epsilon. In some embodiments, a T cell can secrete or can have a membrane bound version of a bispecific antibody which contains an anti-CD3 scFv and an anti-CD2 scFv, thereby bringing the T cell's native CD2 into proximity of the TCR such that when the TCR is engaged/activated, the cell's native CD2 is also activated.

In some embodiments, CD2 signaling in a T cell expressing a transgenic TCR or in bulk tumor infiltrating lymphocytes (TILs) grown ex vivo can be enhanced by several methods. The TILs can then be given back to a patient. For example, the T cells can be transduced with a co-receptor that enhances CD2 signaling (in addition to expressing the transgenic TCR). The co-receptor comprising an extracellular ligand binding domain, a transmembrane domain, and a CD2 signaling domain can be transcribed in a virus or other vector and can provide CD2 signaling in trans even when the target tumor cells express low, absent, or mutated CD58. The extracellular portion of the co-receptor can comprise an scFv recognizing an antigen expressed by the tumor cells or a ligand for a common receptor expressed on the target tumor cell of interest. Another way to enhance CD2 signaling in a CAR T cell, a transgenic TCR T cell, or bulk TILs can comprise transducing the T cells to constitutively express a secreted molecule capable of crosslinking the cell's native CD2 through use of one or more anti-CD2 scFv's, antibodies, Fabs, DARPINs, ligands, or other binders/antigen binding domains. Alternatively, the secreted molecule can be expressed under an activation switch. The secreted molecule can be membrane bound and can consist of two scFv's connected by a linker: one scFv that binds CD2 on the T cell (activating its native CD2 signaling) and the other scFv or ligand recognizing a protein or target expressed on tumor cells such that CD2 is crosslinked and activated when the T cell encounters tumor cells.

### E. Nucleic Acids

An aspect of the disclosure is a nucleic acid that encodes a CD2 CAR, a transgenic TCR, and/or a chimeric polypeptide of the disclosure.

The terms “nucleic acid” and “polynucleotide” are used interchangeably herein, and refer to both RNA and DNA molecules, including nucleic acid molecules comprising cDNA, genomic DNA, and/or synthetic DNA, and DNA or RNA molecules containing nucleic acid analogs. The nucleic acid can comprise one or more bases and/or linkages that do not occur naturally in DNA or RNA, such as phosphoramidite linkages, 2′-modified ribose or deoxyribose, morpholino phosphoramidites, peptide-nucleic acid links, locked nucleic acid links, xanthine, 7-methylguanine, inosine, dihydrouracil, 5-methylcytosine, 5-hydroxymethylcytosine, and others. See, e.g., C. I. E. Smith et al., Ann Rev Pharmacol Toxicol (2019) 59:605-30, incorporated herein by reference. A nucleic acid can be double-stranded or single-stranded (for example, a sense strand or an antisense strand). A nucleic acid may contain unconventional or modified nucleotides. The terms “polynucleotide sequence” and “nucleic acid sequence” as used herein interchangeably refer to the sequence of a nucleic acid molecule. The nomenclature for nucleotide bases set forth in 37 CFR § 1.822 is used herein.

Nucleic acids of the disclosure can encode a CD2 CAR, a transgenic TCR and/or, a chimeric polypeptide. In some embodiments, nucleic acids of the disclosure can encode both a chimeric polypeptide and a CAR (which can be a CD2 CAR or a prior art CAR). In some embodiments, nucleic acids of the disclosure can encode both a chimeric polypeptide and a transgenic TCR. In some embodiments, nucleic acids of the disclosure can encode both a CAR and a transgenic TCR. A nucleic acid that encodes both a chimeric polypeptide, a CAR, and/or a transgenic TCR can be a bicistronic or a tricistronic nucleic acid, wherein the two or three coding sequences are separated by a sequence encoding an IRES (internal ribosome entry site) or a self-cleaving polypeptide sequence such as 2A, which provide for expression of each protein separately, or for the immediate cleavage into two separate proteins upon expression, as shown in Examples 9-11. Examples of 2A sequences include T2A, P2A, E2A, and F2A. In an embodiment, the nucleic acid encodes a CD2 CAR. In some embodiments, separate construts of a CAR and/or a TCR, and a chimeric polypeptide is co-transduced as shown in Examples 12-15. In an embodiment, the nucleic acid encodes a chimeric polypeptide. In some embodiments, the nucleic acid encodes a transgenic TCR. In an embodiment, the nucleic acid is a bicistronic nucleic acid that encodes a CAR and a chimeric polypeptide. In an embodiment, the sequences encoding the CAR and the chimeric polypeptide are separated by a 2A sequence. In an embodiment, the 2A sequence is a P2A sequence. In an embodiment, the nucleic acid encodes a CAR and a chimeric polypeptide. In an embodiment, the nucleic acid encodes a CD2 CAR and a chimeric polypeptide.

In some embodiments, the recombinant nucleic acid is operably linked to a heterologous nucleic acid sequence, such as, for example a structural gene that encodes a protein of interest or a regulatory sequence (e.g., a promoter sequence). In some embodiments, the recombinant nucleic acid is further defined as an expression cassette or a vector. In some embodiments, the vector is a lentiviral vector, an adeno virus vector, an adeno-associated virus vector, or a retroviral vector.

Some embodiments disclosed herein relate to vectors or expression cassettes including a recombinant nucleic acid molecule as disclosed herein. An expression cassettes is a construct of genetic material that contains coding sequences and enough regulatory information to direct proper transcription and/or translation of the coding sequences in a recipient cell, in vivo and/or ex vivo. The expression cassette may be inserted into a vector for targeting to a desired host cell. As such, the term expression cassette may be used interchangeably with the term “expression construct.”

Also provided herein are vectors, plasmids or viruses containing one or more of the nucleic acid molecules encoding any of the CARs and chimeric polypeptides disclosed herein. The nucleic acid molecules described above can be contained within a vector that is capable of directing their expression in, for example, a cell that has been transduced with the vector. Suitable vectors for use in eukaryotic cells are known in the art and are commercially available or readily prepared by a skilled artisan. Additional vectors can also be found, for example, in Ausubel, F. M., et al., Current Protocols in Molecular Biology, (Current Protocol, 1994) and Sambrook et al., “Molecular Cloning: A Laboratory Manual,” 2nd Ed. (1989).

Accordingly, in some embodiments, the CARs, the TCRs, and/or chimeric polypeptides of the present disclosure can be expressed from vectors, generally expression vectors. The vectors are useful for autonomous replication in a host cell or may be integrated into the genome of a host cell upon introduction into the host cell, and thereby are replicated along with the host genome (e.g., non-episomal mammalian vectors). Expression vectors are capable of directing the expression of coding sequences to which they are operably linked. In general, expression vectors of utility in recombinant DNA techniques are often in the form of plasmids (vectors). However, other forms of expression vectors, such as viral vectors (e.g., replication defective retroviruses, adenoviruses, and adeno-associated viruses) are also included.

DNA vectors can be introduced into eukaryotic cells via conventional transformation or transfection techniques. Suitable methods for transforming or transfecting host cells can be found in Sambrook et al. (1989)Molecular Cloning: A Laboratory Manual (2nd ed., Cold Spring Harbor Laboratory Press, Plainview, N.Y.) and other standard molecular biology laboratory manuals.

Vectors suitable for use include the pMSXND expression vector for use in mammalian cells. In some embodiments nucleic acid inserts, which encode the subject CAR and/or TCRs in such vectors, can be operably linked to a promoter, which is selected based on, for example, the cell type in which expression is sought. Viral vectors that can be used in the disclosure include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

In some embodiments, the expression vector can be a viral vector. The term “viral vector” is widely used to refer either to a nucleic acid molecule that includes virus-derived nucleic acid elements that typically facilitate transfer of the nucleic acid molecule or integration into the genome of a cell, or to a viral particle that mediates nucleic acid transfer. Viral particles typically include viral components, and sometimes also host cell components, in addition to nucleic acid(s). Retroviral vectors used herein contain structural and functional genetic elements, or portions thereof, that are primarily derived from a retrovirus. Retroviral lentivirus vectors contain structural and functional genetic elements, or portions thereof including LTRs, that are primarily derived from a lentivirus (a sub-type of retrovirus).

Viral vectors that can be used in the disclosure include, for example, retrovirus vectors (including lentivirus vectors), adenovirus vectors, and adeno-associated virus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

In some embodiments, the nucleic acid molecules are delivered by viral or non-viral delivery vehicles known in the art. For example, the nucleic acid molecule can be stably integrated in the host genome, or can be episomally replicating, or present in the recombinant host cell as a mini-circle expression vector for stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can also be accomplished using classical random genomic recombination techniques or with more precise genome editing techniques such as using guide RNA-directed CRISPR/Cas9, DNA-guided endonuclease genome editing NgAgo (Natronobacterium gregoryi Argonaute), or TALENs genome editing (transcription activator-like effector nucleases). In some embodiments, the nucleic acid molecule is present in the recombinant host cell as a mini-circle expression vector for stable or transient expression.

The nucleic acid molecules can be encapsulated in a viral capsid or a lipid nanoparticle. Alternatively, endonuclease polypeptide(s) can be delivered by viral or non-viral delivery vehicles known in the art, such as electroporation or lipid nanoparticles. For example, introduction of nucleic acids into cells may be achieved using viral transduction methods. In a non-limiting example, adeno-associated virus (AAV) is a non-enveloped virus that can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral systems are also useful for nucleic acid delivery and gene therapy via viral transduction. Lentiviral vectors offer several attractive properties as gene-delivery vehicles, including: (i) sustained gene delivery through stable vector integration into the host cell genome; (ii) the ability to infect both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile (e.g., by targeting a site for integration that has little or no oncogenic potential); and (vii) a relatively easy system for vector manipulation and production.

### F. Engineered Cells

Engineered cells that contain and express a nucleic acid that encodes a CD2 CAR, a transgenic TCR and/or a chimeric polypeptide are also an aspect of the disclosure. An engineered cell of the disclosure is a transduced cell, i.e., a cell into which a nucleic acid molecule, for example a nucleic acid molecule encoding a CAR and/or a transgenic TCR, has been introduced by means of recombinant DNA techniques. The progeny of such a cell are also considered within the scope of the disclosure. Engineered cells of the disclosure are useful for aiding in the treatment of hyperproliferative diseases and disorders such as cancer.

The engineered cells of the disclosure can exhibit improved functional properties, as compared to CAR-T cells lacking the features of the disclosure. For example, an engineered cell of the disclosure having a CAR that comprises a CD2 signaling domain, a transgenic TCR, and/or a chimeric polypeptide can exhibit improved efficacy against target cells that downregulate expression of or do not substantially express CD58; improved efficacy against target cells that downregulate the selected antigen, or express a mutated form of the selected antigen; and/or exhibit improved selectivity for the target cell. In an embodiment, the engineered cell can express a molecule (either secreted or surface expressed) that can crosslink native CD2 in response to a tumor specific antigen in the tumor microenvironment. Improved efficacy against target cells that downregulate expression of or do not substantially express CD58 can be determined by laboratory experiments comparing engineered cells of the disclosure with conventional CAR-T cells, using target cells that express a reduced level of CD58 or a non-functional mutated form of CD58, where improved efficacy can be any demonstration of superior ability to kill or inhibit the target cells. Similar experiments can determine improved efficacy against target cells with down-regulated expression of the selected target antigen. Improved selectivity can be demonstrated by measuring the reduction of on-target off-tumor activity, either in vivo or in a suitable in vitro model.

In some embodiments, host cells can be genetically engineered (e.g., transduced, transformed, or transfected) with, for example, a vector construct of the present disclosure that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule. In some embodiments, the host cell is an immune cell, a stem cell, a mammalian cell, a primate cell, or a human cell. In some embodiments, the host cell is autologous or allogeneic. In some embodiments, the host cell is a T cell, a CD8-positive T cell, a CD4-positive T cell, a regulatory T cell, a cytotoxic T cell, or a tumor infilterating lymphocyte.

Host cells can be transduced with a nucleic acid encoding a CD2 CAR and/or a transgenic TCR, or with one or more nucleic acids encoding a CAR and/or a transgenic TCR plus a chimeric polypeptide. For example, without limitation, a host cell can be transduced with a nucleic acid encoding a CAR and/or a transgenic TCR, and an additional nucleic acid encoding a chimeric polypeptide. In an embodiment, the host cell is transduced with a bicistronic nucleic acid encoding a CAR and a chimeric polypeptide. In an embodiment, the host cell is transduced with a bicistronic nucleic acid encoding a CAR and a transgenic TCR. In an embodiment, the host cell is transduced with a tricistronic nucleic acid encoding a CAR, a transgenic TCR and/or a chimeric polypeptide. In some embodiments, the host cell is further transduced with an additional nucleic acid encoding one or more additional therapeutic agents such as, for example, but not limited to, an antibody, an antibody fragment thereof, or a protein therapeutic capable of stimulating CD2. In some embodiments, a vaccine, an oncoloytic viruse, a checkpoint inhibitor, a T cell agonist antibody, chemotherapy, and/or a bispecific antibody can be combined with CAR T cells or other adoptively transferred T cells.

In some embodiments, the recombinant cell is an animal cell. In some embodiments, the animal cell is a mammalian cell. In some embodiments, the animal cell is a mouse cell. In some embodiments, the animal cell is a human cell. In some embodiments, the recombinant cell is an immune system cell, e.g., a lymphocyte (for example without limitation, a T cell, natural killer cell or NK cell, natural killer T cell or NKT cell, a B cell, a plasma cell, tumor-infiltrating lymphocyte (TIL)), a monocyte or macrophage, or a dendritic cell. In some embodiments, the immune system cell is selected from the group consisting of B cells, T cells, monocytes, dendritic cells, and epithelial cells. In some embodiments, the immune system cell is a T lymphocyte. The immune cell can also be a precursor cell, i.e., a cell that is capable of differentiating into an immune cell.

Techniques for transforming a wide variety of the above-mentioned host cells and species are known in the art and described in the technical and scientific literature. In some embodiments, the nucleic acid molecule is introduced into a host cell by a transduction procedure, electroporation procedure, or a biolistic procedure. Accordingly, cell cultures including at least one recombinant cell as disclosed herein are also within the scope of this application. Methods and systems suitable for generating and maintaining cell cultures are known in the art. An aspect of the disclosure is the method for making an engineered cell, by transducing the cell with a nucleic acid that encodes a CD2 CAR and/or a chimeric polypeptide of the disclosure, in such a manner that the nucleic acid is expressed.

In a related aspect, some embodiments of the disclosure relate to a cell culture including at least one recombinant cell as disclosed herein, and a culture medium. Generally, the culture medium can be any one of suitable culture media for the cell cultures described herein. In some embodiments, the recombinant cell expresses a CAR and/or chimeric polypeptide described herein.

### G. Antibodies

An aspect of the disclosure is an antibody or an antigen binding fragment thereof for use with a CAR and/or a transgenic TCR in a CAR-T therapy. In some embodiments, the antibody or the antigen binding fragment thereof is multispecific. In some embodiments, a multispecific antibody is bispecific. In an embodiment, the antibody or the antigen binding fragment thereof is bispecific (e.g., a tumor specific antigen or tumor-associated antigen and CD2). In some embodiments, a multispecific antibody is trispecific. In an embodiment, the antibody or the antigen binding fragment thereof is trispecific (e.g., a tumor specific antigen or tumor-associated antigen, CD2, and CD3). In some embodiments, the tumor specific antigen or tumor-associated antigen is selected from the group consisting of glioma-associated antigen, carcinoembryonic antigen (CEA), beta-human chorionic gonadotropin, alpha-fetoprotein (AFP), lectin-reactive AFP, thyroglobulin, RAGE-1, MN-CA IX, human telomerase reverse transcriptase, RU1, RU2 (AS), intestinal carboxyl esterase, mut HSP70-2, M-CSF, prostate-specific antigen (PSA), PAP, NY-ESO-1, LAGE-1a, p53, prostein, PSMA, HER2, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, GD2, GD3, B7-H3, GPC2, L1CAM, EGFR, mesothelin, MART-1, gp100 (Pmel 17), tyrosinase, TRP-1, TRP-2, MAGE-1, MAGE-3, BAGE, GAGE-1, GAGE-2, p15, p53, Ras, HER-2, BCR-ABL, E2A-PRL, H4-RET, IGH-IGK, MYL-RAR, EBVA, human papillomavirus (HPV) antigens E6 and E7, TSP-180, MAGE-4, MAGE-5, MAGE-6, RAGE, NY-ESO-1, p185erbB2, p180erbB-3, c-met, nm-23H1, PSA, TAG-72, CA 19-9, CA 72-4, CAM 17.1, NuMa, K-ras, β-Catenin, CDK4, Mum-1, p15, p16, 43-9F, 5T4, 791Tgp72, α-fetoprotein, β-HCG, BCA225, BTAA, CA125, BCAA, CA195, CA242, CA-50, CAM43, CD68/P1, CO-029, FGF-5, G250, Ga733/EpCAM, HTgp-175, M344, MA-50, MG7-Ag, MOV18, NB/70K, NY-CO-1, RCAS1, SDCCAGI6, TA-90, TAAL6, TAG72, TLP, TPS CD19, CD20, CD22, ROR1, and GD2.

Native antibodies and native immunoglobulins (Igs) can be heterotetrameric glycoproteins of about 150,000 Daltons, composed of two identical light chains and two identical heavy chains. Antibodies can further refer to camelid antibodies, which can be not tetrameric. Each light chain can be typically linked to a heavy chain by one covalent disulfide bond, while the number of disulfide linkages can vary among the heavy chains of different immunoglobulin isotypes. Each heavy and light chain can have regularly spaced intrachain disulfide bridges. Each heavy chain can have at one end a variable domain (“V_{H}”) followed by a number of constant domains (“C_{H}”). Each light chain can have a variable domain at one end (“V_{L}”) and a constant domain (“C_{L}”) at its other end; the constant domain of the light chain can be aligned with the first constant domain of the heavy chain, and the light-chain variable domain can be aligned with the variable domain of the heavy chain. Particular amino acid residues can form an interface between the light- and heavy-chain variable domains.

In some instances, an antibody or an antigen-binding fragment thereof includes an isolated antibody or antigen-binding fragment thereof, a purified antibody or antigen-binding fragment thereof, a recombinant antibody or antigen-binding fragment thereof, a modified antibody or antigen-binding fragment thereof, or a synthetic antibody or antigen-binding fragment thereof. Antibodies and antigen-binding fragments herein can be partly or wholly synthetically produced. An antibody or antigen-binding fragment can be a polypeptide or protein having a binding domain which can be, or can be homologous to, an antigen binding domain. In some instances, an antibody or an antigen-binding fragment thereof can be produced in an appropriate in vivo animal model and then isolated and/or purified.

Antibodies useful in the present disclosure can encompass monoclonal antibodies, polyclonal antibodies, chimeric antibodies, bispecific antibodies, multispecific antibodies, heteroconjugate antibodies, humanized antibodies, human antibodies, deimmunized antibodies, mutants thereof, fusions thereof, immunoconjugates thereof, antigen-binding fragments thereof, and/or any other modified configuration of the immunoglobulin molecule that includes an antigen recognition site of the required specificity, including glycosylation variants of antibodies, amino acid sequence variants of antibodies, and covalently modified antibodies.

Any of the antibodies herein can be multispecific. In an embodiment, a multispecific antibody can be trispecific (e.g., an anti-tumor antigen, CD2, and CD3). In another embodiment, a multispecific antibody can be bispecific (e.g., an anti-tumor antigen and CD2). Bispecific antibodies can be antibodies that have binding specificities for at least two different antigens and can be prepared using the antibodies disclosed herein. Exemplary methods for making bispecific antibodies are described (see, e.g., Suresh et al., 1986, Methods in Enzymology 121:210). The recombinant production of bispecific antibodies can be based on the co-expression of two immunoglobulin heavy chain-light chain pairs, with the two heavy chains having different specificities (Millstein and Cuello, 1983, Nature, 305, 537-539). Bispecific antibodies can be composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, can facilitate separation of the desired bispecific compound from unwanted immunoglobulin chain combinations.

Functional fragments of any of the antibodies herein are also contemplated. The terms “antigen-binding portion of an antibody,” “antigen-binding fragment,” “antigen-binding domain,” “antibody fragment,” or a “functional fragment of an antibody” can refer to one or more fragments of an antibody that retain the ability to specifically bind to an antigen. Representative antigen-binding fragments include a Fab, a Fab′, a F(ab′)₂, a Fv, a scFv, a dsFv, a variable heavy domain, a variable light domain, a variable NAR domain, bi-specific scFv, a bi-specific Fab₂, a tri-specific Fab₃, an AVIMER®, a minibody, a diabody, a triabody, a maxibody, a camelid, a VHH, a minibody, an intrabody, fusion proteins comprising an antibody portion (e.g., a domain antibody), and a single chain binding polypeptide.

“F(ab′)₂” and “Fab′” moieties can be produced by treating an Ig with a protease such as pepsin and papain, and include antibody fragments generated by digesting immunoglobulin near the disulfide bonds existing between the hinge regions in each of the two heavy chains. For example, papain can cleave IgG upstream of the disulfide bonds existing between the hinge regions in each of the two heavy chains to generate two homologous antibody fragments in which an light chain composed of V_{L} and C_{L} (light chain constant region), and a heavy chain fragment composed of V_{H} and C_{Hγ1} (γ1) region in the constant region of the heavy chain) are connected at their C terminal regions through a disulfide bond. Each of these two homologous antibody fragments can be called Fab′. Pepsin can also cleave IgG downstream of the disulfide bonds existing between the hinge regions in each of the two heavy chains to generate an antibody fragment slightly larger than the fragment in which the two above-mentioned Fab′ are connected at the hinge region. This antibody fragment can be called F(ab′)₂.

The Fab fragment can also contain the constant domain of the light chain and the first constant domain (C_{H}1) of the heavy chain. Fab′ fragments can differ from Fab fragments by the addition of a few residues at the carboxyl terminus of the heavy chain C_{H}1 domain including one or more cysteine(s) from the antibody hinge region. Fab′-SH can be a Fab′ in which the cysteine residue(s) of the constant domains bear a free thiol group. F(ab′)₂ antibody fragments can be produced, for example, as pairs of Fab′ fragments which have hinge cysteines between them. Other chemical couplings of antibody fragments can also be employed.

A “Fv” as used herein can refer to an antibody fragment which contains a complete antigen-recognition and antigen-binding site. This region can consist of a dimer of one heavy chain and one light chain variable domain in tight, non-covalent or covalent association (disulfide linked Fvs have been described, see, e.g., Reiter et al. (1996) Nature Biotechnology 14:1239-1245). In this configuration that the three CDRs of each variable domain can interact to define an antigen-binding site on the surface of the V_{H}-V_{L} dimer. Collectively, a combination of one or more of the CDRs can from each of the V_{H} and V_{L} chains confer antigen-binding specificity to the antibody. For example, the CDRH3 and CDRL3 can be sufficient to confer antigen-binding specificity to an antibody when transferred to V_{H} and V_{L} chains of a recipient antibody or antigen-binding fragment thereof and this combination of CDRs can be tested for binding, specificity, affinity, etc. using, for example, techniques described herein. In some cases, even a single variable domain (or half of an Fv comprising only three CDRs specific for an antigen) can have the ability to recognize and bind antigen, although likely at a lower specificity or affinity than when combined with a second variable domain. Furthermore, although the two domains of a Fv fragment (V_{L} and V_{H}) can be coded for by separate genes, they can be joined using recombinant methods, for example by a synthetic linker that enables them to be made as a single protein chain in which the V_{L} and V_{H} regions pair to form monovalent molecules (known as single chain Fv (scFv); Bird et al. (1988) Science 242:423-426; Huston et al. (1988) Proc. Natl. Acad. Sci. USA 85:5879-5883; and Osbourn et al. (1998) Nat. Biotechnol. 16:778). Such scFvs can be encompassed within the term “antigen-binding portion” of an antibody. Any V_{H} and V_{L} sequences of specific scFv can be linked to an Fc region cDNA or genomic sequences in order to generate expression vectors encoding complete Ig (e.g., IgG) molecules or other isotypes. V_{H} and V_{L} can also be used in the generation of Fab, Fv, or other fragments of Igs using either protein chemistry or recombinant DNA technology.

“Single-chain Fv” or “sFv” antibody fragments can include the V_{H} and V_{L} domains of an antibody, wherein these domains can be present in a single polypeptide chain. In some embodiments, the Fv polypeptide can further include a polypeptide linker between the V_{H} and V_{L} domains which enables the sFv to form the desired structure for antigen binding. For a review of sFvs, see, e.g., Pluckthun in The Pharmacology of Monoclonal Antibodies, Vol. 113, Rosenburg and Moore eds. Springer-Verlag, New York, pp. 269-315 (1994).

As used herein, a “dsFv” can be a Fv fragment obtained, for example, by introducing a Cys residue into a suitable site in each of a heavy chain variable region and a light chain variable region, and then stabilizing the heavy chain variable region and the light chain variable region by a disulfide bond. The site in each chain, into which the Cys residue can be introduced, can be determined based on a conformation predicted by molecular modeling. In the present disclosure, for example, a conformation can be predicted from the amino acid sequences of the heavy chain variable region and light chain variable region of the above-described antibody, and DNA encoding each of the heavy chain variable region and the light chain variable region, into which a mutation has been introduced based on such prediction, can be then constructed. The DNA construct can be incorporated then into a suitable vector and prepared from a transformant obtained by transformation with the aforementioned vector.

Diabodies can be single chain antibodies. Diabodies can be bivalent, bispecific antibodies in which VH and VL domains can be expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al., Proc. Natl. Acad. Sci. USA, 90:6444-6448 (1993); and Poljak, R. J., et al., Structure, 2:1121-1123 (1994)).

### H. Methods of Treatment

Engineered cells of the disclosure are used to aid in the therapy of a hyperproliferative disorder, for example a cancer. Administration of engineered cells (or nucleic acids for generating engineered cells in situ), alone or in combination with other agents (e.g., an antibody or an antigen binding fragment thereof, or a molecule (administered, secreted, or surface expressed) that can crosslink native CD2 in response to a tumor specific antigen in the tumor microenvironment), aids in the treatment or therapy by reducing the number and/or severity of symptoms experienced by a subject, increasing overall or long-term survival, killing pathological cells such as tumor cells or other hyperproliferative cells, reducing the tumor burden, inhibiting the growth of tumor cells or other hyperproliferative cells, inhibiting the spread or proliferation of tumor cells or other hyperproliferative cells, and the like. In some embodiments, a vaccine, an oncoloytic viruse, a checkpoint inhibitor, a T cell agonist antibody, chemotherapy, and/or a bispecific antibody can be combined with CAR T cells or other adoptively transferred T cells.

Methods for administering immune cells for therapy are known and may be used in connection with the provided methods and compositions. For example, adoptive T cell therapy methods are described in US 2003/0170238; U.S. Pat. No. 4,690,915; S. A. Rosenberg, Nat Rev Clin Oncol (2011) 8(10):577-85. See also M. Themeli et al., Nat Biotechnol (2013) 31(10):928-33; and T. Tsukahara et al., Biochem Biophys Res Commun (2013) 438(1):84-89. In an aspect of the disclosure, the method comprises administering a CAR-T cell of the disclosure.

In some embodiments, therapeutic agents described herein, e.g., engineered CAR-Ts and CAR-T cells with chimeric polypeptides and/or a transgenic TCR, can be used in methods of treating individuals who have, who are suspected of having, or who may be at high risk for developing a cancer. In some embodiments, the cancer under expresses CD58, or expresses a mutated form of CD58 that can no longer ligate CD2. In some embodiments, the cancer is a leukemia. In these instances, the leukemia can generally be of any type of leukemia. In some embodiments, the cancer is a lymphoma. In some embodiments, the cancer is acute lymphoblastic leukemia (ALL), chronic lymphocytic leukemia (CLL), B-cell prolymphocytic leukemia, precursor B lymphoblastic leukemia, hairy cell leukemia, diffuse large B-cell lymphoma (DLBCL), follicular lymphoma, marginal zone lymphoma, mantle cell lymphoma, Burkitt's lymphoma, MALT lymphoma, Waldenstrom's macroglobulinemia, or another disorder characterized by the overgrowth of B-lineage cells.

In other embodiments, the tumor is a solid tumor cancer. In some embodiments, the solid tumor cell is lung cancer, liver cancer, pancreatic cancer, stomach cancer, colon cancer, kidney cancer, brain cancer, head and neck cancer, breast cancer, skin cancer, rectal cancer, uterine cancer, cervical cancer, ovarian cancer, testicular cancer, skin cancer, or esophageal cancer. In some embodiments, the cancer includes a sarcoma cell, a rhabdoid cancer cell, a neuroblastoma cell, retinoblastoma cell, or a medulloblastoma cell. In some embodiments, the cancer is uterine carcinosarcoma (UCS), brain lower grade glioma (LGG), thymoma (THYM), testicular germ cell tumors (TGCT), glioblastoma multiforme (GBM) and skin cutaneous melanoma (SKCM), liver hepatocellular carcinoma (LIHC), uveal melanoma (UVM), kidney chromophobe (KICH), thyroid cancer (THCA), kidney renal clear cell carcinoma (KIRC), kidney renal papillary cell carcinoma (KIRP), stomach adenocarcinoma (STAD), cholangiocarcinoma (CHOL), adenoid cystic carcinoma (ACC), prostate adenocarcinoma (PRAD), pheochromocytoma and paraganglioma (PCPG), DLBC, lung adenocarcinoma (LUAD), head-neck squamous cell carcinoma (HNSC), pancreatic adenocarcinoma (PAAD), breast cancer (BRCA), mesothelioma (MESO), colon and rectal adenocarcinoma (COAD), rectum adenocarcinoma (READ), esophageal carcinoma (ESCA), ovarian cancer (OV), lung squamous cell carcinoma (LUSC), bladder urothelial carcinoma (BLCA), sarcoma (SARC), or uterine corpus endometrial carcinoma (UCEC). In some embodiments, the administered first therapeutic agent inhibits tumor growth or metastasis of the cancer in the subject. In some embodiments, the cancer includes a metastatic cancer cell, a multiply drug resistant cancer cell, or a recurrent cancer cell. In some embodiments, the administered first therapeutic agent confers increased production of interferon gamma (IFNγ) and/or interleukin-2 (IL-2) in the subject. In some embodiments, the cancer has reduced expression of CD58. In some embodiments, the cancer is uterine carcinosarcoma (UCS), brain lower grade glioma (LGG), thymoma (THYM), testicular germ cell tumors (TGCT), glioblastoma multiforme (GBM), or skin cutaneous melanoma (SKCM).

An effective amount of the engineered cells described herein is determined based on the intended goal, for example tumor regression. For example, where existing cancer is being treated, the amount of a therapeutic agent disclosed herein to be administered may be greater than where administration of the therapeutic agent is for prevention of cancer. One of ordinary skill in the art will be able to determine the amount of a therapeutic agent to be administered and the frequency of administration in view of this disclosure. The quantity to be administered, both according to number of treatments and dose, also depends on the individual to be treated, the state of the individual, and the protection desired. Precise amounts of the therapeutic agent also depend on the judgment of the practitioner and can be peculiar to each individual. Frequency of administration could range from 1-2 days, to 2-6 hours, to 6-10 hours, to 1-2 weeks or longer depending on the judgment of the practitioner.

In certain embodiments of the present disclosure, the therapeutic agents will be an aqueous composition that includes the engineered cells described herein. Aqueous compositions of the present disclosure contain an effective amount of a therapeutic agent disclosed herein in a pharmaceutically acceptable carrier or aqueous medium. Thus, the “pharmaceutical preparation” or “pharmaceutical composition” of the disclosure can include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the recombinant cells disclosed herein, its use in the manufacture of the pharmaceutical compositions is contemplated. Supplementary active ingredients can also be incorporated into the compositions. For human administration, preparations should meet sterility, pyrogenicity, general safety, and purity standards as required by the FDA Center for Biologics.

The engineered cells described herein can be used to cure established tumors, inhibit tumor growth or metastasis of cancer in the treated subject relative to the tumor growth or metastasis in subjects who have not been administered one of the therapeutic compositions disclosed herein. In some embodiments, the engineered cells can be used to stimulate immune responses against the tumor via inducing the production of interferon gamma (IFNγ) and/or interleukin-2 (IL-2), and other pro-inflammatory cytokines. The production of interferon gamma (IFNγ) and/or interleukin-2 (IL-2) can be stimulated to produce up to about 20 fold, such as any of about 2 fold, 3 fold, 4 fold, 5 fold, 6 fold, 7 fold, 8 fold, 9 fold, 10 fold, 11 fold, 12 fold, 13 fold, 14 fold, 15 fold 16 fold, 17 fold, 18 fold, 19 fold, or 20 fold or higher compared to the production of interferon gamma (IFNγ) and/or interleukin-2 (IL-2) in subjects who have not been administered one of the therapeutic compositions disclosed herein.

As discussed herein, engineered cells can be administered in combination with one or more additional therapeutic agents such as, for example, chemotherapeutics or anti-cancer agents or anti-cancer therapies, antibodies or antigen binding fragments thereof, or a molecule (administered, secreted, or surface expressed) that can crosslink native CD2 in response to a tumor specific antigen in the tumor microenvironment. Administration “in combination with” one or more additional therapeutic agents includes simultaneous (concurrent) and consecutive administration in any order. In some embodiments, the one or more additional therapeutic agents, chemotherapeutics, anti-cancer agents, or anti-cancer therapies is selected from the group consisting of chemotherapy, radiotherapy, immunotherapy, hormonal therapy, toxin therapy, and surgery. “Chemotherapy” and “anti-cancer agent” are used interchangeably herein. Various classes of anti-cancer agents can be used. Non-limiting examples include: alkylating agents, antimetabolites, anthracyclines, plant alkaloids, topoisomerase inhibitors, podophyllotoxin, antibodies (e.g., monoclonal or polyclonal), checkpoint inhibitors, immunomodulators, cytokines, nanoparticles, radiation therapy, tyrosine kinase inhibitors (for example, imatinib mesylate), hormone treatments, soluble receptors and other antineoplastics. In some embodiments, a therapeutic agent is a secreted, surface expressed, or administered molecule that is capable of crosslinking cell's native CD2 in response to a tumor specific antigen expressed in the tumor microenvironment. In some embodiments, T cells can be transduced to constitutively express a secreted molecule capable of crosslinking the cell's native CD2 through use of one or more anti-CD2 scFv's, antibodies, Fabs, DARPINs, ligands, or other binders/antigen binding domains. Alternatively, the secreted molecule can be expressed under an activation switch. The secreted molecule can be membrane bound and can consist of two scFv's connected by a linker: one scFv that binds CD2 on the T cell (activating its native CD2 signaling) and the other scFv or ligand recognizing a protein or target expressed on tumor cells such that CD2 is crosslinked and activated when the T cell encounters tumor cells.

Topoisomerase inhibitors are also another class of anti-cancer agents that can be used herein. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins: irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

Antineoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell's DNA.

Alkylating agents can alkylate many nucleophilic functional groups under conditions present in cells. Cisplatin and carboplatin, and oxaliplatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinblastine, vinorelbine, and vindesine.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are obtained from plants and block cell division by preventing microtubule function. Since microtubules are vital for cell division, without them, cell division cannot occur. The main examples are vinca alkaloids and taxanes. Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

Podophyllotoxin is a plant-derived compound which has been reported to help with digestion as well as used to produce two other cytostatic drugs, etoposide and teniposide. They prevent the cell from entering the G1 phase (the start of DNA replication) and the replication of DNA (the S phase).

In some embodiments, the anti-cancer agents can be selected from remicade, docetaxel, celecoxib, melphalan, dexamethasone (Decadron@), steroids, gemcitabine, cisplatinum, temozolomide, etoposide, cyclophosphamide, temodar, carboplatin, procarbazine, gliadel, tamoxifen, topotecan, methotrexate, gefitinib (Iressa®), taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib (Velcade®), bisphosphonate, arsenic trioxide, vincristine, doxorubicin (Doxil®), paclitaxel, ganciclovir, adriamycin, estrainustine sodium phosphate (Emcyt®), sulindac, etoposide, and combinations of any thereof.

In other embodiments, the anti-cancer agent can be selected from bortezomib, cyclophosphamide, dexamethasone, doxorubicin, interferon-alpha, lenalidomide, melphalan, pegylated interferon-α, prednisone, thalidomide, or vincristine.

In some embodiments, the methods of treatment as described herein further include administration of a compound that inhibits one or more immune checkpoint molecules. In some embodiments, the one or more immune checkpoint molecules include one or more of CTLA4, PD-1, PD-L1, A2AR, B7-H3, B7-H4, TIM3, and combinations of any thereof. In some embodiments, the compound that inhibits the one or more immune checkpoint molecules includes an antagonistic antibody. In some embodiments, the antagonistic antibody is ipilimumab, nivolumab, pembrolizumab, durvalumab, atezolizumab, tremelimumab, or avelumab.

In some aspects, the one or more anti-cancer therapy is radiation therapy. In some embodiments, the radiation therapy can include the administration of radiation to kill cancerous cells. Radiation interacts with molecules in the cell such as DNA to induce cell death. Radiation can also damage the cellular and nuclear membranes and other organelles. Depending on the radiation type, the mechanism of DNA damage may vary as does the relative biologic effectiveness. For example, heavy particles (protons and neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray. Radiation also contemplated herein includes, for example, the directed delivery of radioisotopes to cancer cells. Other forms of DNA damaging factors are also contemplated herein such as microwaves and UV irradiation.

Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a fractioned regime. For example, the regime may include fractionated individual doses of 2 Gy. Dosage ranges for radioisotopes vary widely, and depends on the half-life of the isotope and the strength and type of radiation emitted. When the radiation includes use of radioactive isotopes, the isotope may be conjugated to a targeting agent, such as a therapeutic antibody, which carries the agent to the target tissue (e.g., tumor tissue).

Surgery described herein includes resection in which all or part of a cancerous tissue is physically removed, exercised, and/or destroyed. Tumor resection refers to physical removal of at least part of a tumor. In addition to tumor resection, treatment by surgery includes laser surgery, cryosurgery, electrosurgery, and micropically controlled surgery (Mohs surgery). Removal of precancers or normal tissues is also contemplated herein.

Accordingly, in some embodiments, the methods of the disclosure further include administering to the individual a second therapeutic agent, such as an anti-cancer agent, a chemotherapeutic, or anti-cancer therapy. In some embodiments, the second anti-cancer agent or anti-cancer therapy is selected from the group consisting of chemotherapy, radiotherapy, immunotherapy, hormonal therapy, toxin therapy, and surgery. In some embodiments, the first therapeutic agent and the second anti-cancer agent or therapy are administered concomitantly. In some embodiments, the first therapeutic agent and the second anti-cancer agent or therapy are administered sequentially. In some embodiments, the first therapeutic agent is administered before the second anti-cancer agent or therapy. In some embodiments, the first therapeutic agent or therapy is administered before and/or after the second anti-cancer agent or therapy. In some embodiments, the first therapeutic agent and the second anti-cancer agent or therapy are administered in rotation. In some embodiments, the first therapeutic agent is administered at the same time as the second anti-cancer agent or therapy. In some embodiments, the first therapeutic agent and the second anti-cancer agent or therapy are administered together in a single formulation.

In some embodiments, the expression of functional CD58 by the target cells (e.g., tumor cells) is determined prior to administering engineered cells of the disclosure. The ability of engineered cells of the disclosure to function against target cells with low expression of functional CD58 (for example, target cells that express low amounts or no CD58, or express CD58 that is mutated such that it no longer effectively ligates CD2) makes them a therapy of choice when target cells exhibit such characteristics. In an embodiment, a determination of functional CD58 expression level on the target cell(s) is provided prior to administering engineered cells. The determination can be provided by a third part. In an embodiment, engineered cells are administered only if the functional CD58 expression level falls below a threshold value. In an embodiment, the threshold value is about 50,000, 45,000, 40,000, 35,000, 30,000, 25,000, 20,000, 15,000, 14,000, 13,000, 12,000, 11,000, 10,000, 9,000, 8,000, 7,000, 6,000, 5,000, 4,000, 3,000, 2,000, 1,000, or 500 functional CD58 molecules per target cell (that are capable of ligating CD2). The expression level of functional CD58 can be determined by standard methods, for example flow cytometry or mass cytometry, employing antibodies specific for a functional epitope of CD58 (see, e.g., T. J. Dengler et al., Eur J Immunol (1992) 22(11):2809-17). The expression level can also be determined using a T cell activation assay, for example by measuring the amount of IL-2 and/or IFNγ produced by a CAR-T cell in response to contact with a target cell that expresses the CAR target antigen (see, e.g., Example 6 below): these results can be calibrated against model target cells (e.g., nalm6 cells) that express CD58 at different known levels (e.g., as determined by flow cytometry). This approach is useful for determining the expression level in cases wherein the target cell expresses a mutated form of CD58 that has impaired CD2-binding ability, as an equivalent to the expression level of fully functional CD58. For example, if the target cell induces a degree of CAR-T cell activation most similar to the activation induced by a model cell that expresses 11,000 molecules of CD58 per cell, then 11,000 would be the functional CD58 expression level for purposes of determining threshold expression, regardless of the actual number of CD58 molecules expressed by the target cell (which, due to mutation, may be more or less able to activate CD2). In some embodiments, loss of CD58 can significantly reduce IFNγ and IL-2 production in CAR T treatment, resulting in reduced efficacy of CAR T cells. In some embodiments, the CAR provided herein is not affected by loss of CD58 or rescue the effect of CD58 loss. In some embodiments, the CAR provided herein comprises CD2 and can outperform the conventional CAR without CD2 regardless of CD58 status. In some embodiments, the CAR provided herein enhances tumor control and survival compared to the conventional CAR without CD2. In some embodiments, the CAR provided herein performs comparably in terms of cytotoxicity. In some embodiments, adding a CD2 co-stimulatory domain to the 4-1BB co-stimulatory domain improves the cytokine release against CD58 loss. In some embodiments, trans CAR and CD2 construct(s) performs better than cis CAR and CD2 construct. In some embodiments, trans CAR co-expressed with CD2 containing receptor recognizing CD20 can rescue CAR T cell function against CD58 loss. In some embodiments, trans CAR co-expressed with CD2 containing receptor recognizing CD20 can rescue CAR T cell function against CD58 loss and maintain activity against cells that lost the target antigen (e.g., CD19).

### I. Systems

Another aspect of the disclosure is a system for aiding in the treatment of a subject in need thereof, by administering an engineered cell of the disclosure. The subject, in general, will have been diagnosed as having, or at risk of having a hyperproliferative disorder characterized by the proliferation of a target cell having at least a first antigen. The engineered cell expresses a CD2 CAR, or a CAR in combination with a transgenic TCR and/or a chimeric polypeptide, that is specific for the first antigen. In some embodiments, the target cells exhibit decreased or absent expression of CD58, or express a mutant CD58 that no longer effectively ligates CD2. The system further includes a labeled binding agent, for determining the state of CD58 present on the target cells. This enables the practitioner to determine when use of the engineered cells of the disclosure would be preferred over the use of another agent. As the engineered cells of the disclosure are capable of functioning and killing target cells that express little or no wild-type CD58, they are particularly indicated when the target cells are determined to express little or no wild-type CD58.

In an embodiment, the labeled binding agent comprises a labeled antibody or antibody derivative, or a chemical compound that specifically binds CD58. In an embodiment, the labeled binding agent specific for CD58 comprises a labeled antibody. In an embodiment, the labeled binding agent is soluble CD2 protein. In an embodiment, the label comprises a radioactive atom that can be imaged radiographically. In an embodiment, the label comprises a fluorescent molecule. In an embodiment, the label comprises a luminescent molecule. In an embodiment, the label comprises a colorimetric molecule. In an embodiment, the label comprises a binding agent such as biotin, avidin, or streptavidin that is capable of binding another detectable molecule (such as a radio-labeled avidin). Labeled binding agent of the disclosure can be used in vivo or ex vivo.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art.

Such techniques are explained fully in the literature cited herein.

Additional embodiments are disclosed in further detail in the following examples, which are provided by way of illustration and are not in any way intended to limit the scope of this disclosure or the claims.

Materials and Methods

The following methods and materials were used in the Examples described below.

1. Synthesis of Chimeric Antigen Receptors (CARs) and Chimeric Polypeptides

Genes encoding scFv's were synthesized as either gene fragments (gBlock, IDT DNA) or gene-encoding plasmids synthesized by GeneArt (Life Technologies), and then cloned into a MSGV1 retroviral expression vectors using restriction cloning (Roche) or In-fusion cloning (Takara). CARs having CD19-BBz or CD22-BBz were constructed having a CD8α hinge domain and CD8a transmembrane domain. CARs having CD19-28z were constructed having a CD28 hinge and transmembrane domain. CARs having CD2 signaling domains were constructed having a CD8α hinge and transmembrane domain.

2. Retroviral Vector Production and T-Cell Transduction

Retroviral supernatant was produced via transient transfection of the 293GP packaging cell line as previously described. Briefly, 70% confluent cells were co-transfected via Lipofectamine® 2000 (Life Technologies) in 150 mm Poly-D-Lysine culture dishes with the plasmids encoding the CARs and the RD 114 envelope protein. Media was replaced at 24 and 48 hours post transfection. Viral supernatant was harvested 48 and 72 hours post-transfection and centrifuged to remove cell debris and stored at −80° C. until use.

Primary human T cells were isolated from healthy donors using the RosetteSep Human T cell Enrichment kit (Stem Cell Technologies), using buffy coats obtained from the Stanford Blood Center and processed according to the manufacturer's protocol using Lymphoprep® density gradient medium and SepMate-50 tubes. Isolated T cells were cryopreserved in CryoStor CS10 cryopreservation medium (Stem Cell Technologies). Cryopreserved T cells were thawed and activated with Human T-Expander CD3/CD28 Dynabeads (Gibco) at a 3:1 beads:cell ratio in AIM-V media supplemented with 5% FBS, 10 mM HEPES, 2 mM L-glutamine, 100 U/mL penicillin, and 100 μg/mL streptomycin (Gibco) and with 100 IU/ml of recombinant IL-2 (Preprotech). T cells were transduced with retroviral vector on days 2 and 3 post activation and anti-CD3/CD28 beads were removed on day 5. Car T-cells were maintained at 0.3-1×10⁶ cells per mL in T cell medium with IL-2. CAR expression was assessed by Flow Cytometry after incubation with soluble, recombinant, human CD19 or CD22 labelled with Dylight650. CAR-T cells were used for in vitro assays or transferred into mice on day 10 post activation.

3. ELISA

Cytokine release was assayed by co-incubating 1×10⁵ CAR+ T cells and 1×10⁵ tumor cells in complete RPMI-1640 in triplicates. At 24 hours, culture media were collected and cytokines were measured using IFNγ and IL-2 MAbs (BioLegend).

4. IncuCyte® Killing Assays

For IncuCyte® incubator killing assays, 5×10⁴ GPF-positive tumor cells were plated in triplicates in 96-well flat-bottom plates and co-incubated with CAR-positive T-cells or an equivalent number of control CAR T cells at 1:1 effector to target ratios in 200 μl RPMI-1640. Plates were imaged every 2-3 hours using the IncuCyte® ZOOM Live-Cell analysis system (Essen Bioscience) and 4 images per well at 10× zoom were collected at each time point. Total integrated GFP intensity per well was assessed as a quantitative measure of viable, GFP-positive tumor cells. Values were normalized to the starting measurement and plotted over time.

### Example 1: Impairment of CAR-T Cytokine Release by CD58 Deletion

This experiment was performed to determine the effect of CD58 absence on CAR-T cytokine release.

Three CARs were constructed: CD19-28z (SEQ ID NOs: 63, 64), CD19-BBz (SEQ ID NOs: 65, 66), and CD22-BBz (m971-BBz; SEQ ID NOs: 59, 60), using standard techniques, and were transduced into primary human T cells to provide CAR T cells. Nalm6 cells (a B cell leukemia line) expressing CRISPR Cas9 were used with or without a guide RNA (gRNA) specific for CD58, to knock out CD58 expression in one Nalm6 group (Nalm6 CD58KO), or provide a control Nalm6 group (Nalm6 Cas9).

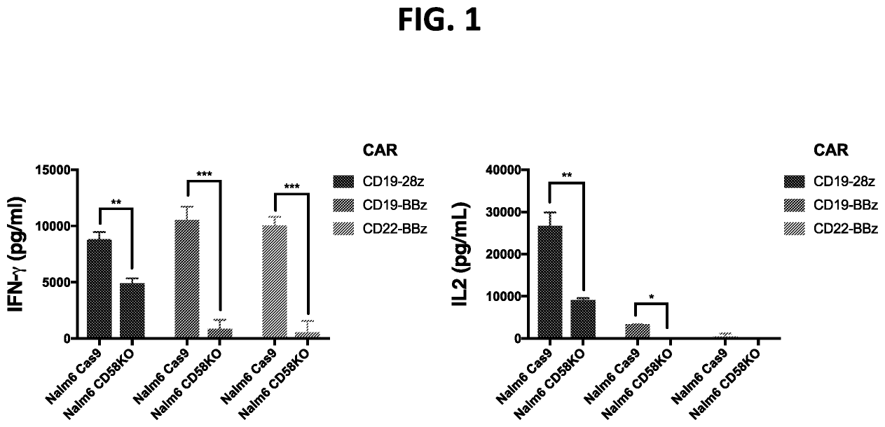

Each CAR group was cocultured for 24 hours with 100,000 target Nalm6 cells at a 1:1 ratio, then IFNγ and IL-2 were measured in the culture supernatant by ELISA. As shown in FIG. 1, deletion of CD58 significantly reduced IFNγ production in all three CAR T constructs, and significantly reduced IL-2 production in CD19-28z and CD19-BBz CAR T (CD22-BBz did not produce significant amounts of IL-2 with or without CD58).

### Example 2: Impairment of CD22 CAR-T Cytotoxicity by CD58 Deletion

This experiment was performed to determine the effect of CD58 absence on CD22 CAR-T activity.

CD22 CART cells were prepared with either of two CARs: m971-BBz (SEQ ID NOs: 59, 60) and m971-28z (SEQ ID NOs: 61, 62), each using the m971 anti-CD22 scFv (SEQ ID NO: 2). Nalm6 target cells with and without CD58 expression were engineered to express GFP, enabling quantification of tumor cell killing by GFP fluorescence.

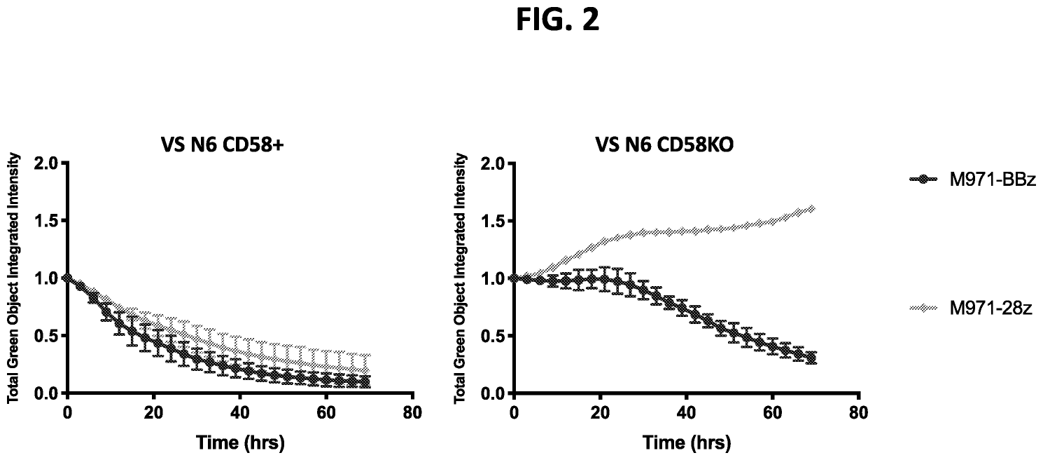

Each CAR T group was incubated for 72 hours with 50,000 target cells at a 1:1 ratio. Fluorescence over time was measured in an IncuCyte® Zoom Live-Cell Analysis System. As shown in FIG. 2, both CAR Ts had reduced efficacy against the CD58 knock out (CD58KO) cells.

### Example 3: Impairment of CD19 CAR-T Cytotoxicity by CD58 Deletion

This experiment was performed to determine the effect of CD58 absence on CD19 CAR-T activity, and under varying levels of CD19 expression. Cell lines used as targets in vitro to stimulate CAR-T cells often express high levels of antigen, such as CD19. This level of CD19 expression does not necessarily reflect the expression of CD19 often found in subjects with lymphoma, as tumor cells can down-regulate expression of the target antigen (and/or experience a selection pressure against target antigen expression). This down-regulation results in decreased CAR-T activity in conventional CAR-T configurations. Accordingly, in this example, Nalm6 cell lines were modified to express CD19 at two reduced expression levels in order to test the CAR-T cells of the disclosure under conditions that more closely resemble clinical circumstances.

CD19 CAR T cells were prepared with either of two CARs: CD19-BBz (SEQ ID NOs: 65, 66) and CD19-28z (SEQ ID NOs: 63, 64), each using an anti-CD19 scFv (SEQ ID NO: 1). Nalm6 target cells with and without CD58 expression were engineered to express GFP, enabling quantification of tumor cell killing by GFP fluorescence. The wildtype Nalm6 target cells express an estimated 45,000 copies of CD19 per cell, without further modification. Additional target cells were engineered to express only 6,196 copies of CD19 per cell (on average) or 963 copies per cell (on average).

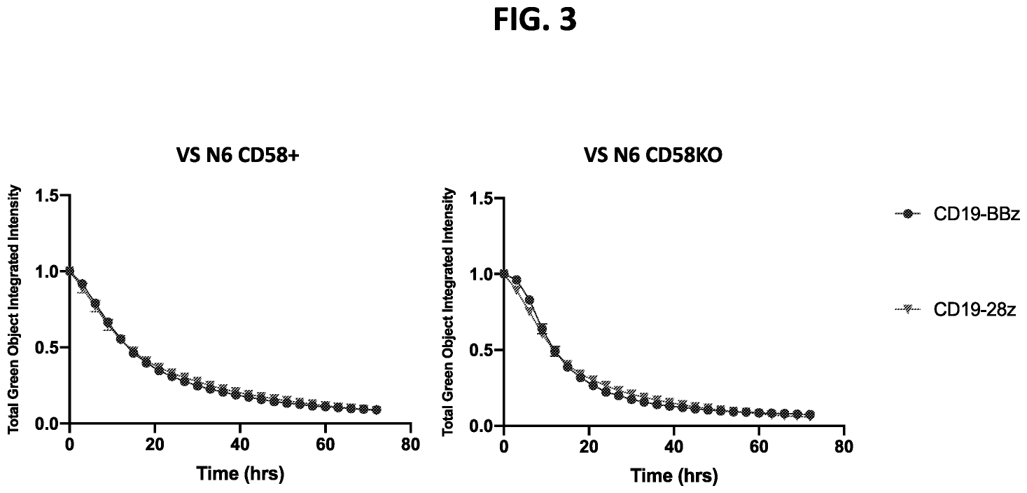

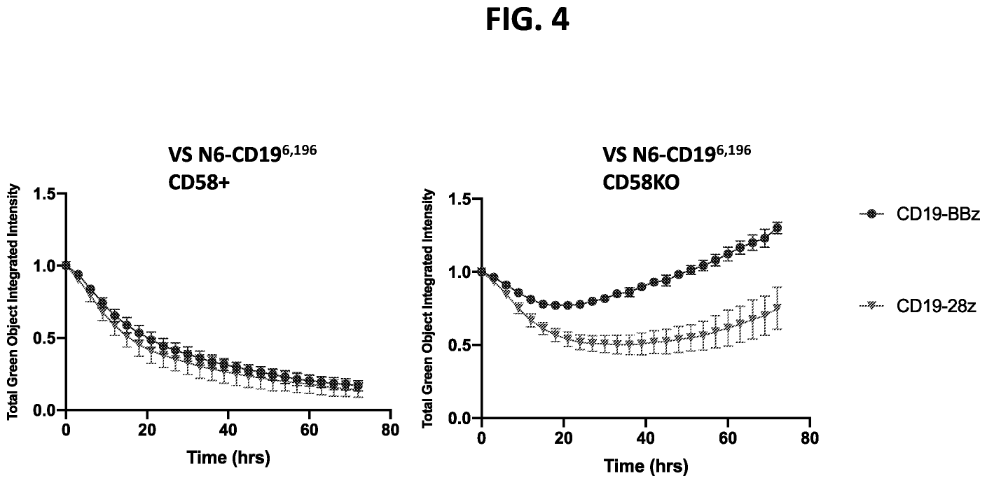

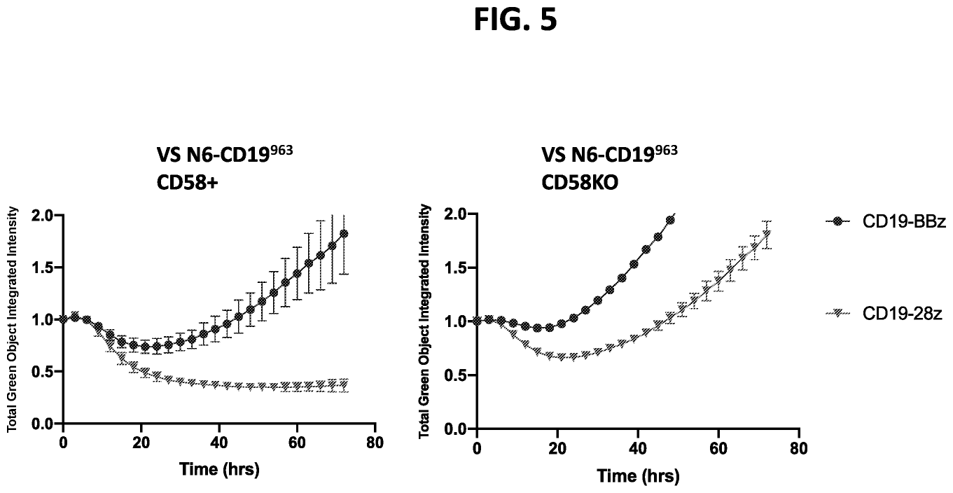

Each CAR T group was incubated for 72 hours with 50,000 target cells from each target group at a 1:1 ratio. Fluorescence over time was measured in an IncuCyte® Zoom Live-Cell Analysis System. As shown in FIG. 3, both CAR-Ts were not affected by CD58KO at 45,000 CD19/cell, but both had had reduced efficacy against the CD58 knock out (CD58KO) cells at lower levels of CD19 expression. FIGS. 4 and 5 show that CARs have decreasing efficacy as CD19 expression is decreased to 6,196 and 963 CD19 molecules per cell, respectively, and that CD58 absence has a greater effect.

### Example 4: Impairment of CD22 CAR-T Cytotoxicity In Vivo

This experiment was performed to demonstrate tumor escape from conventional CD22 CAR-T cell treatment in the absence of CD58 expression.

Nalm6 cells were engineered to express luciferase, and CD58 expression was knocked out in one group. Mice (N=10 per group) were inoculated with 1 million cells of either Nalm6 Cas9 (electroporated with Cas9 without a gRNA, CD58⁺), or Nalm6-CD58KO (electroporated with Cas9 with a CD58 specific gRNA, CD58⁻). After three days, half of each group received either 3,000,000 CD22-41BBz (SEQ ID NOs: 59, 60) CAR T cells, or 3,000,000 mock (untransduced) T cells. Total luminesce flux (tumor BLI) was measured 1-2 times per week.

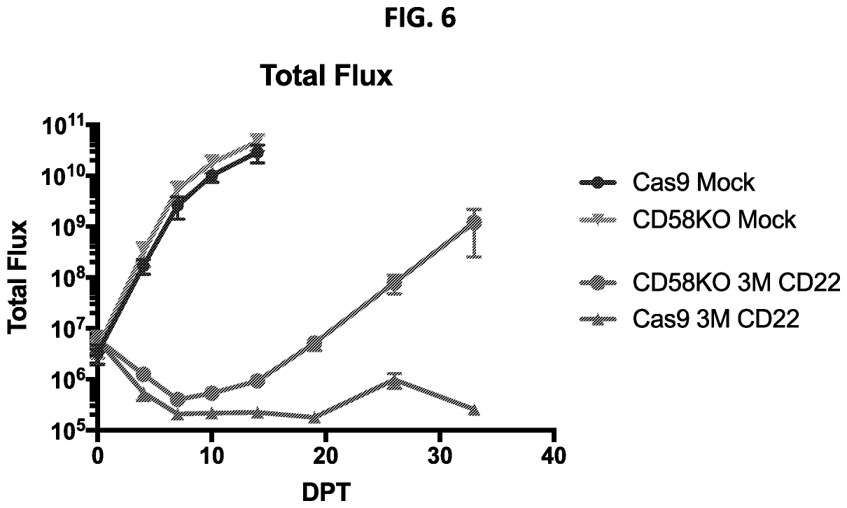

As shown in FIG. 6, tumors grew rapidly in all mice receiving mock-transduced T cells (Cas9 Mock and CD58KO Mock). Conventional m971-BBz CAR T cells were able to control tumor growth of CD58⁺ cells (Cas9 3M CD22), but achieved only a transient response with CD58⁻ cells (CD58KO 3M CD22).

### Example 5: Impairment of CD19 CAR-T Cytotoxicity In Vivo

This experiment was performed to demonstrate tumor escape from conventional CD19 CAR-T cell treatment in the absence of CD58 expression.

Nalm6 cells were engineered to express luciferase, and CD58 expression was knocked out in one group. Mice (N=10 per group) were inoculated with 1 million cells of either Nalm6 Cas9 (expressing Cas9 without a gRNA, CD58⁺), or Nalm6-CD58KO (CD58⁻). After three days, half of each group received either 3,000,000 CD19-28z (SEQ ID NOs: 63, 64) CAR T cells, or 3,000,000 mock (untransduced) T cells. Total luminesce flux (tumor BLI) was measured 1-2 times per week.

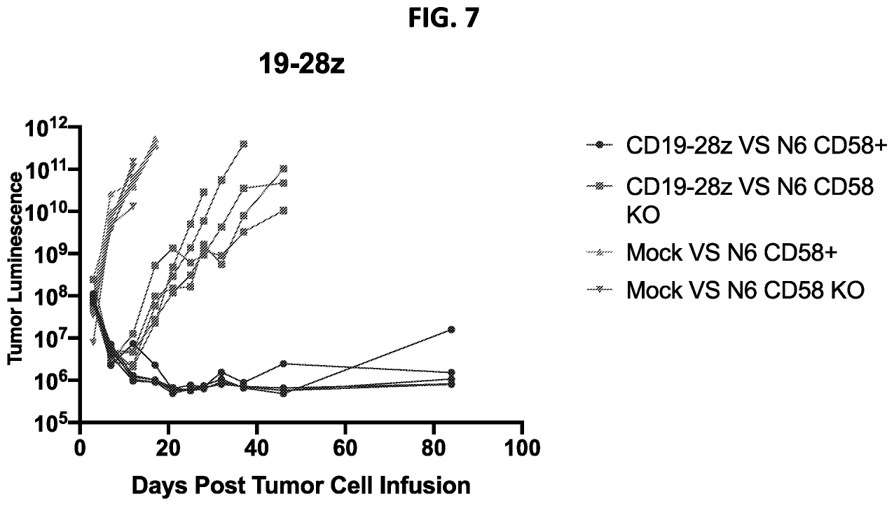

As shown in FIG. 7, tumors grew rapidly in all mice receiving mock-transduced T cells. Conventional CD19-28z CAR T cells were able to control tumor growth of CD58⁺ cells (CD19-28z VS N6 CD58+), but achieved only a transient response with CD58⁻ cells (CD19-28z VS N6 CD58 KO).

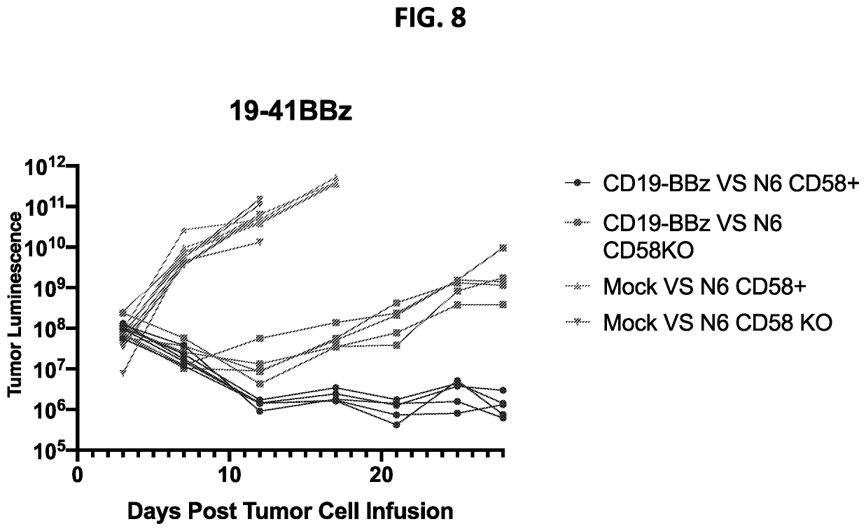

The experiment was repeated with conventional CD19-BBz (SEQ ID NOs: 65, 66) CAR-T cells, with similar results, as shown in FIG. 8.

### Example 6: CD22 CD2 CARs

This experiment was conducted to compare CD22 CARs with and without CD2 domains against CARs with and without 4-1BB domains.

The following CARs were constructed, using the anti-CD22 scFv m971 (SEQ ID NOs: 2, 31) and a CD8α transmembrane domain (SEQ ID NOs: 5, 34): m971-BBz (SEQ ID NOs: 59, 60), m971-CD2-BBz (SEQ ID NOs: 17, 46), m971-CD2-z (SEQ ID NOs: 16, 45), m971-BB-CD2z (SEQ ID NOs: 18, 47), and m971-BBz-CD2 (SEQ ID NOs: 67, 68). These CARs were transduced into T cells as described above.

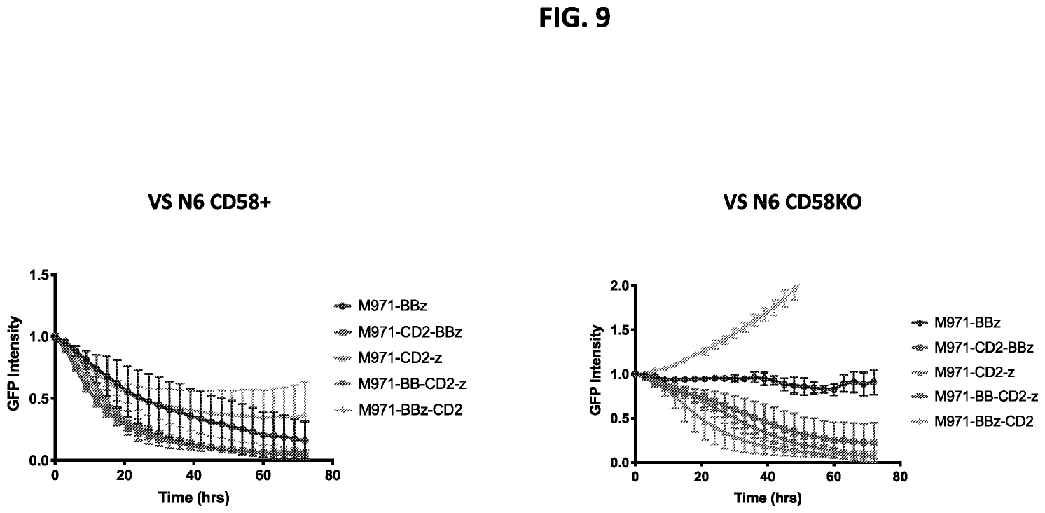

Each of the CAR-Ts (50,000 cells) was incubated with 50,000 Nalm6 tumor cells (GFP⁺, either CD58⁺ or CD58⁻) for 72 hours in an IncuCyte® incubator. As shown in FIG. 9, m971-BBz-CD2 was not effective, but all other CD2-containing CARs (m971-CD2-BBz; m971-CD2-z; and m971-BB-CD2-z) outperformed the conventional m971-BBz CAR against both CD58⁺ and CD58⁻ cells.

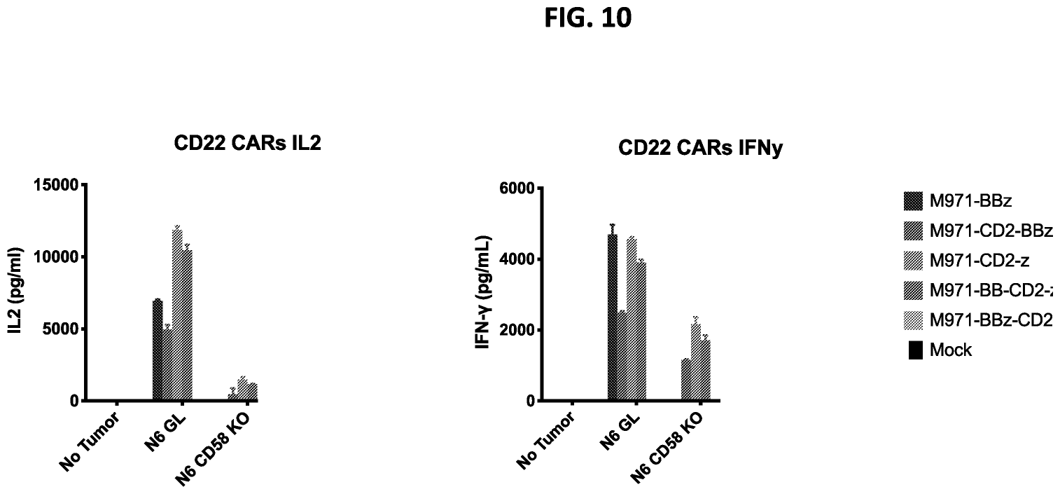

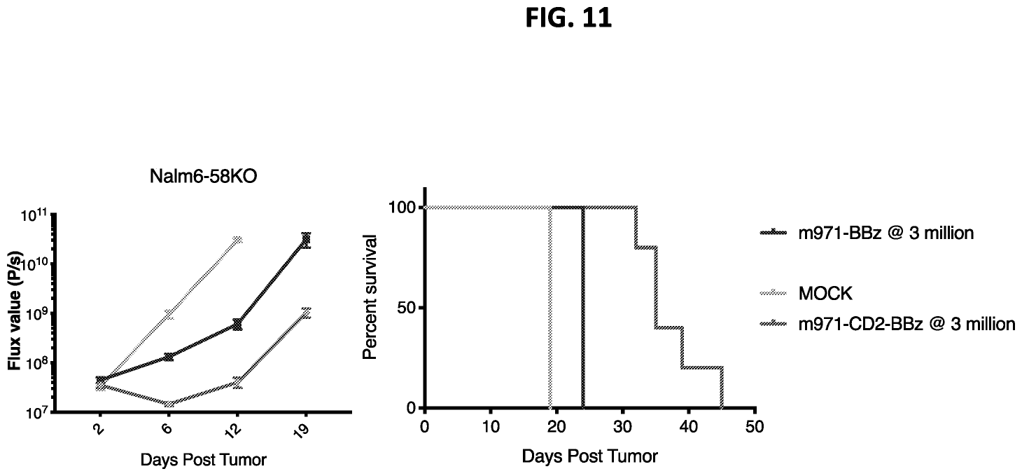

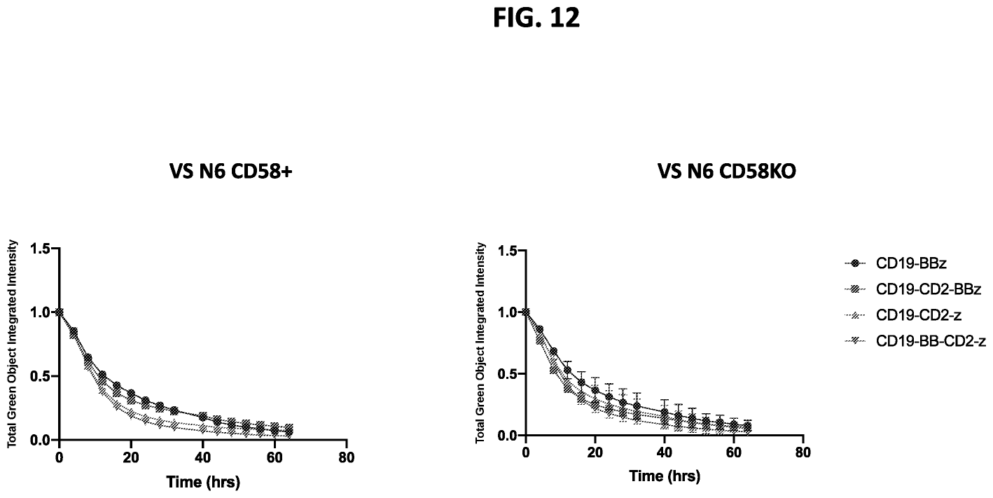

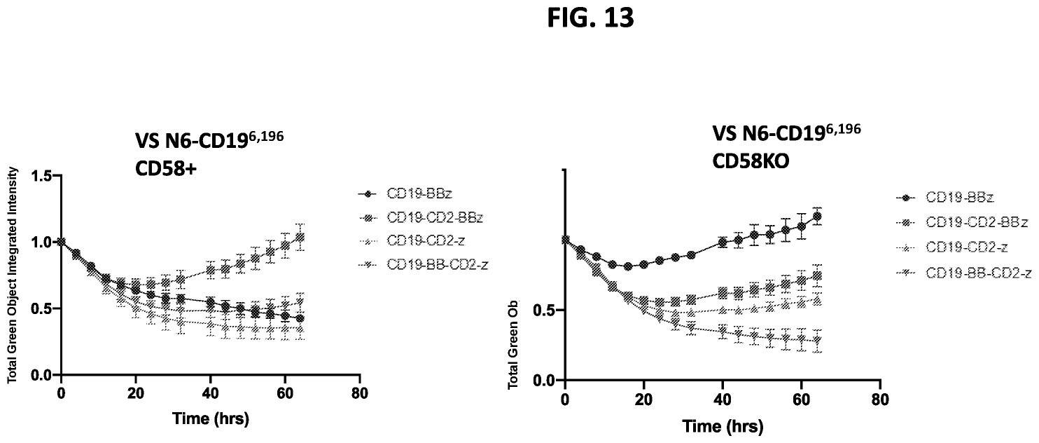

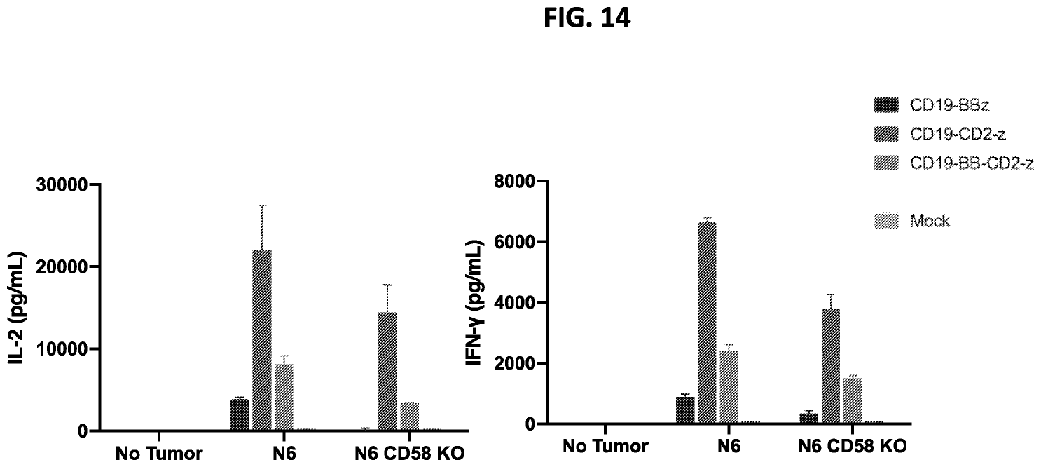

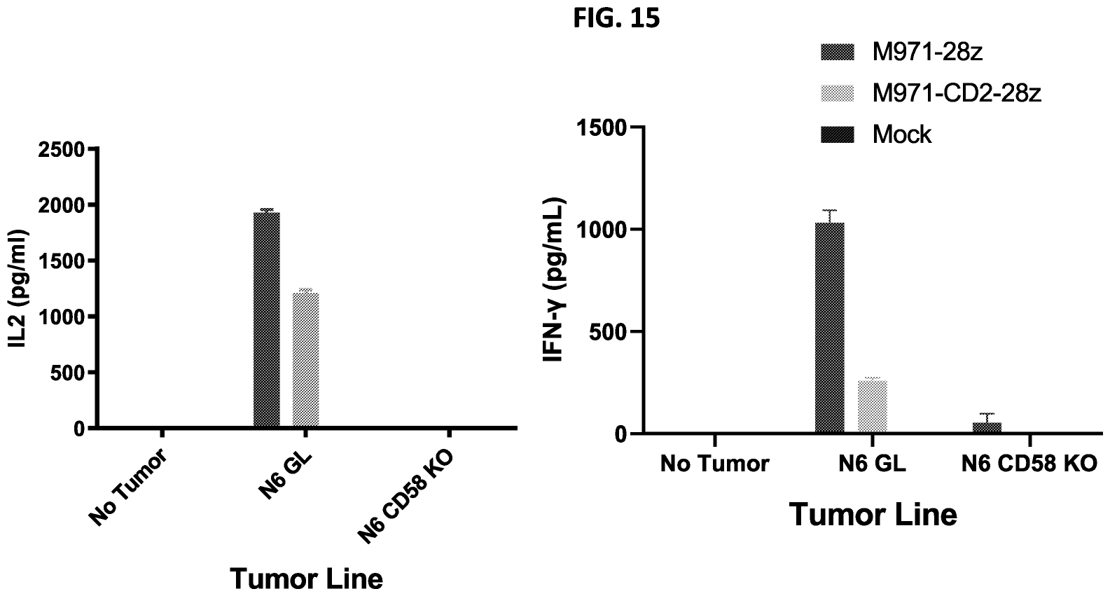

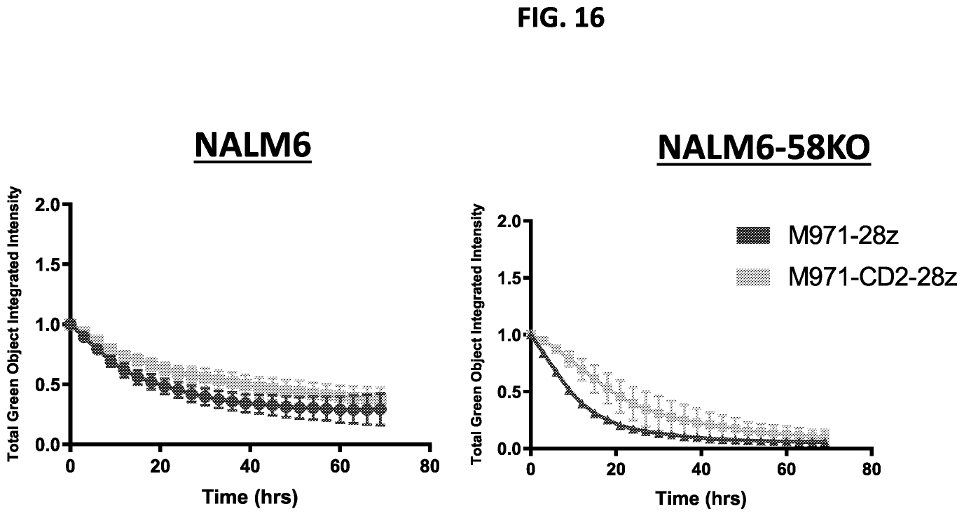

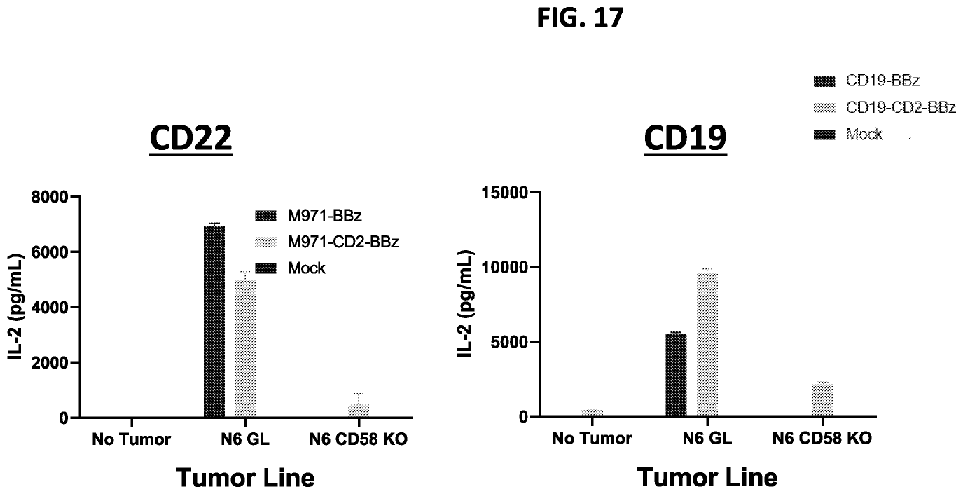

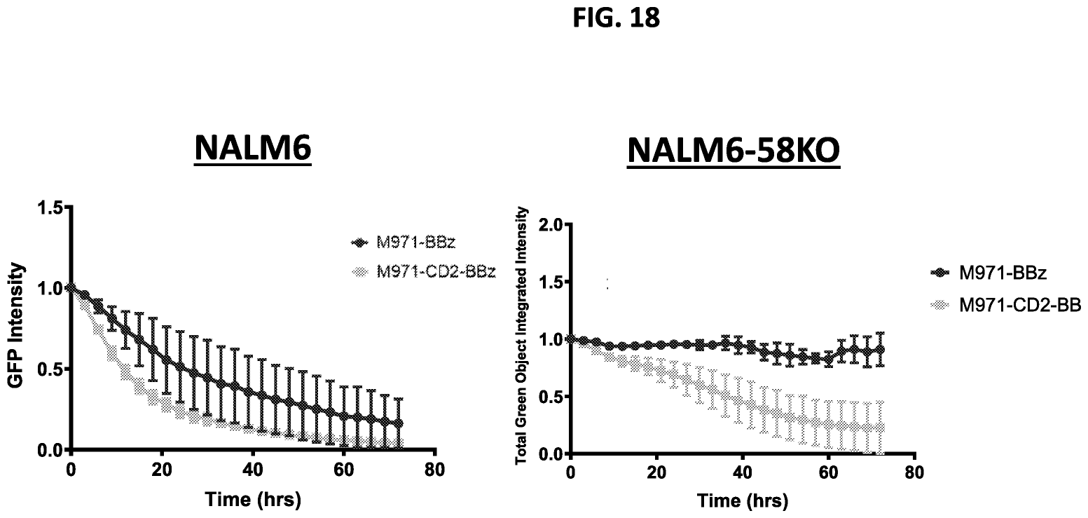

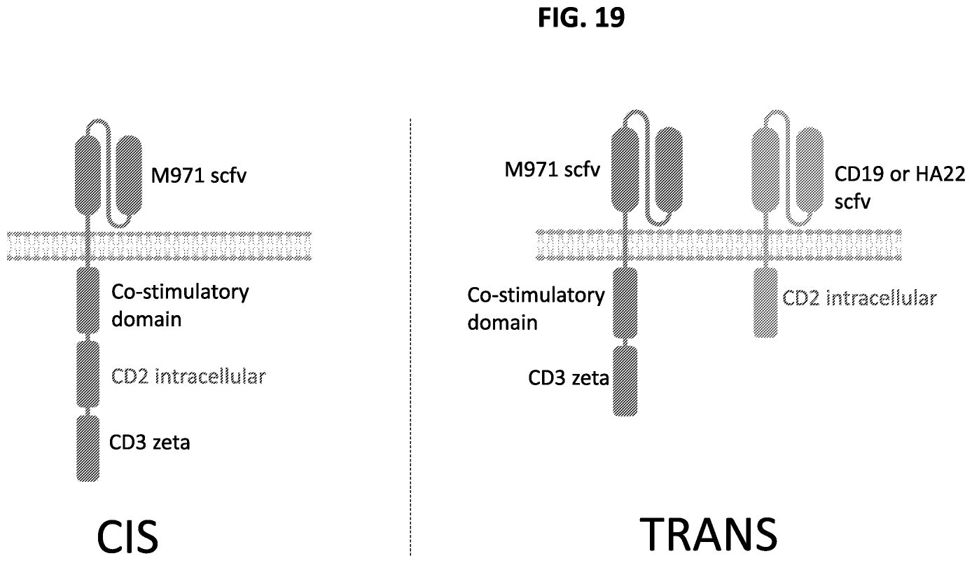

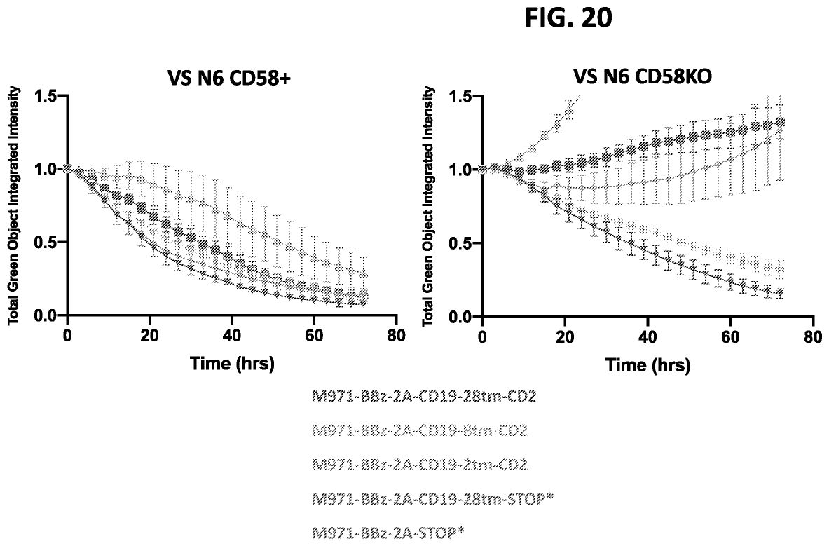

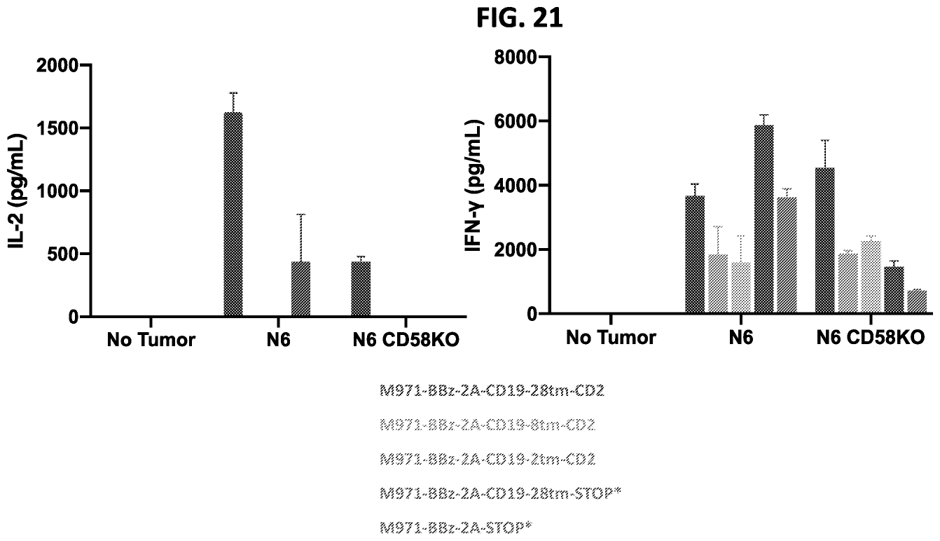

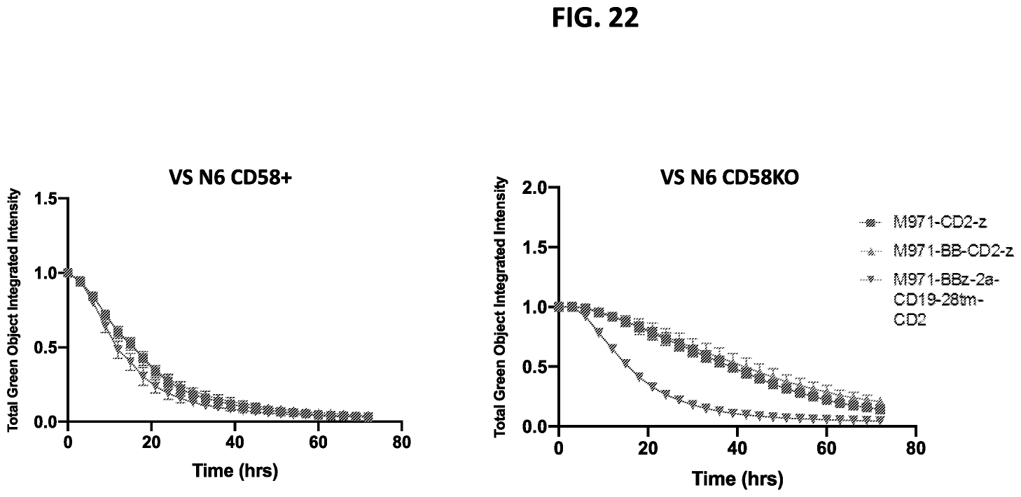

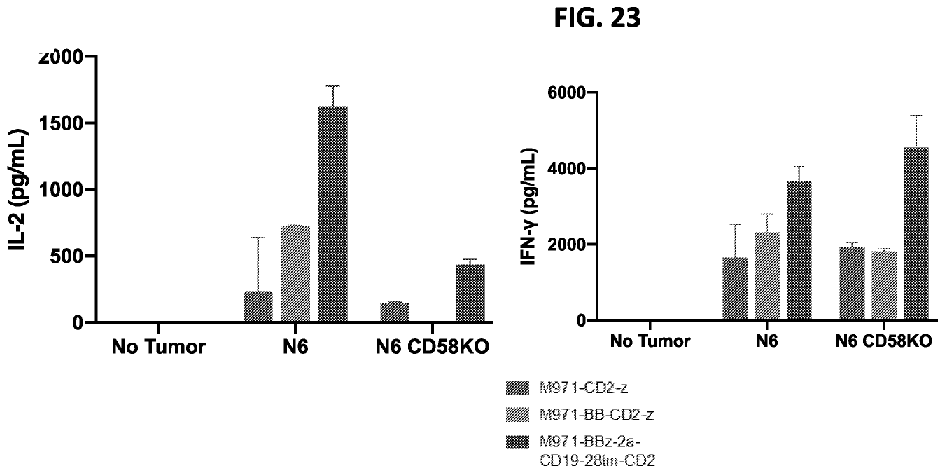

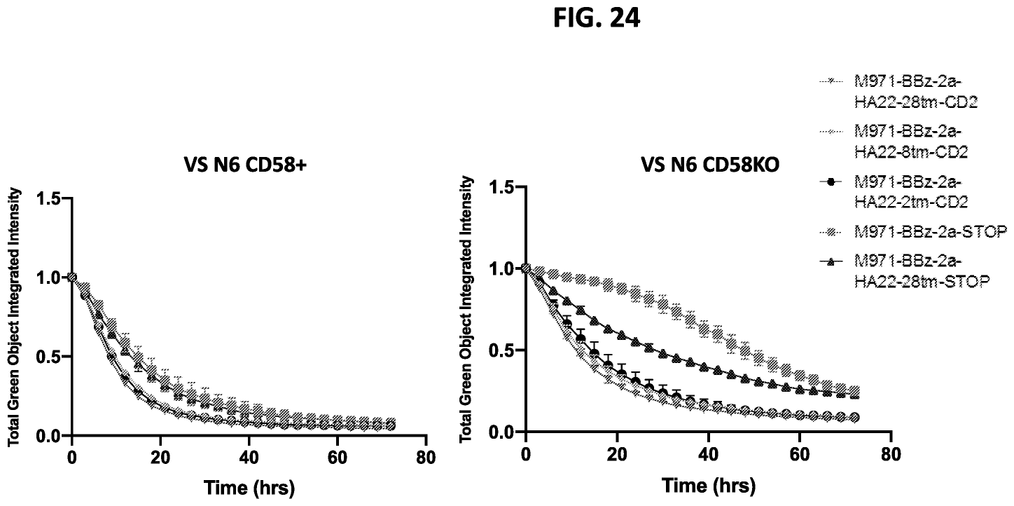

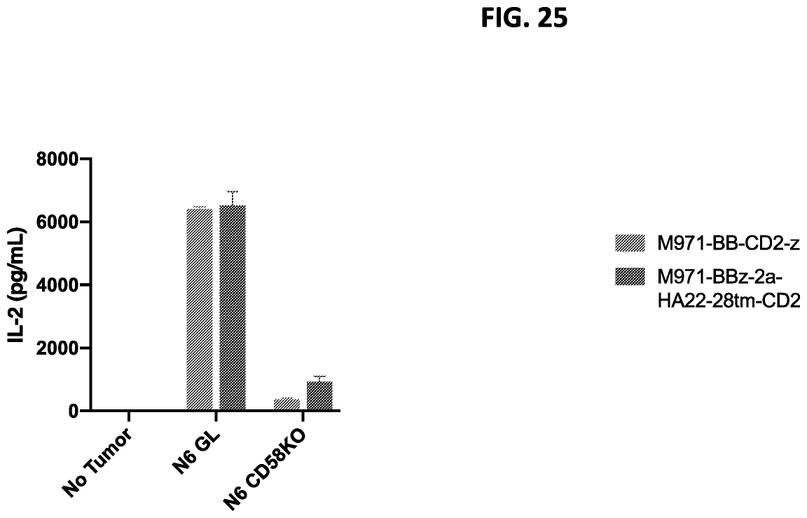

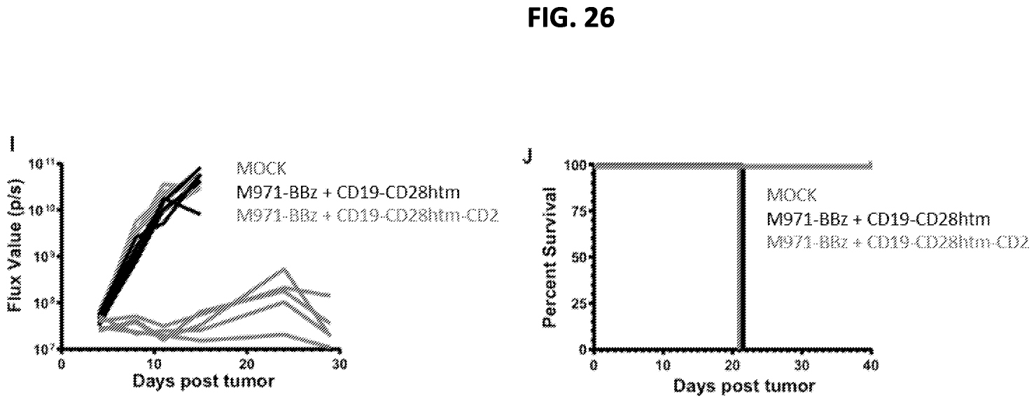

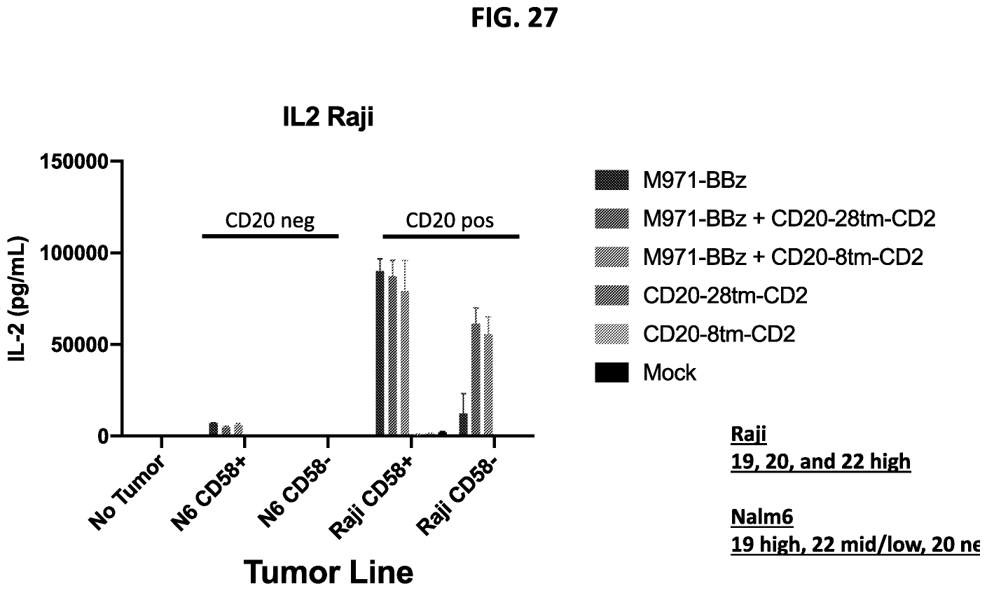

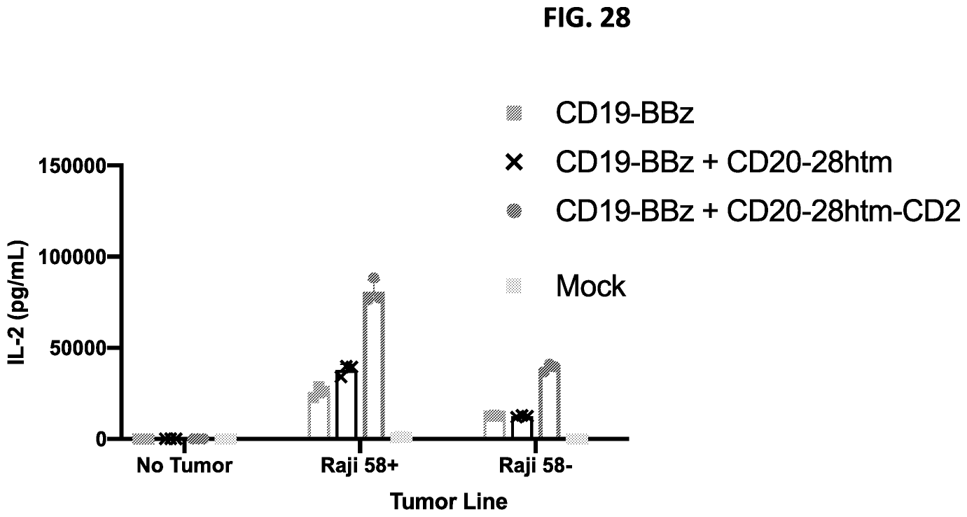

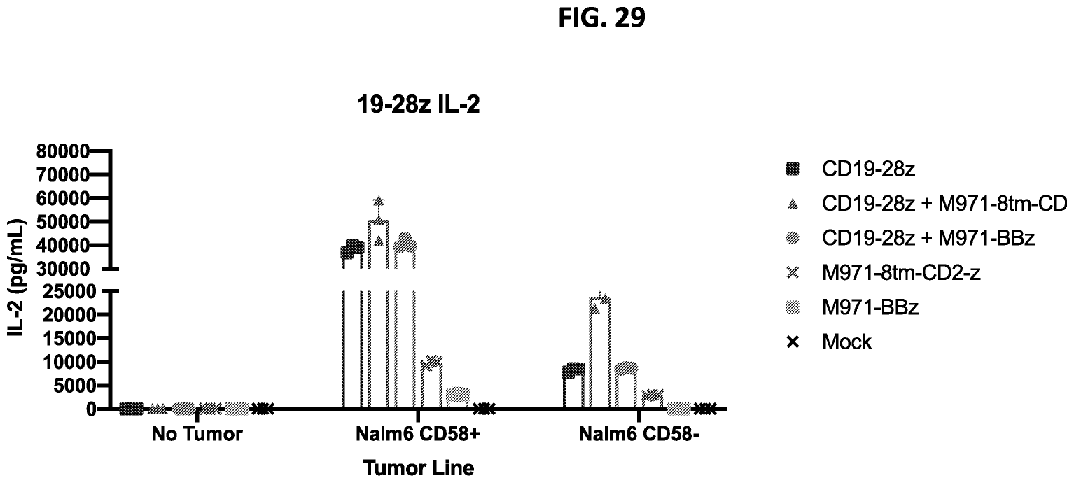

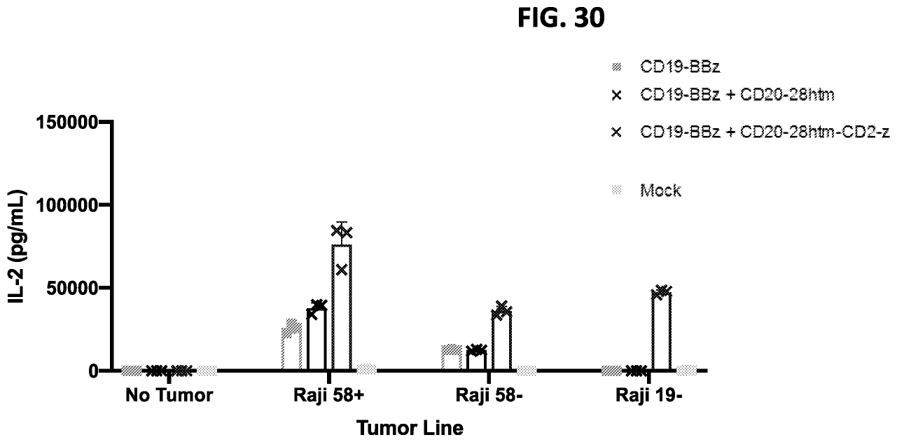

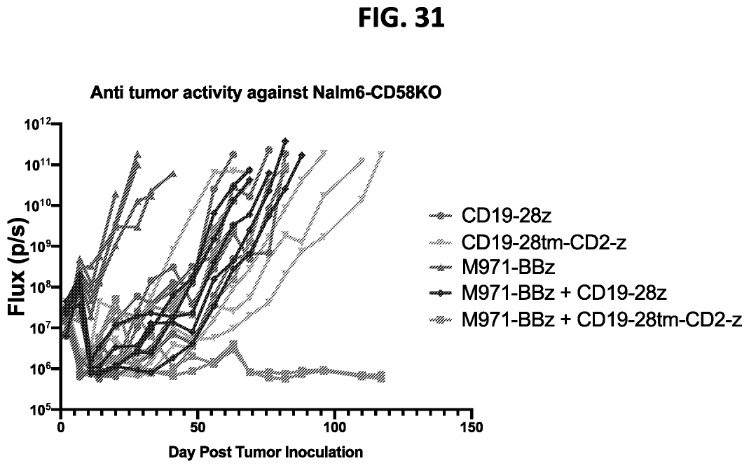

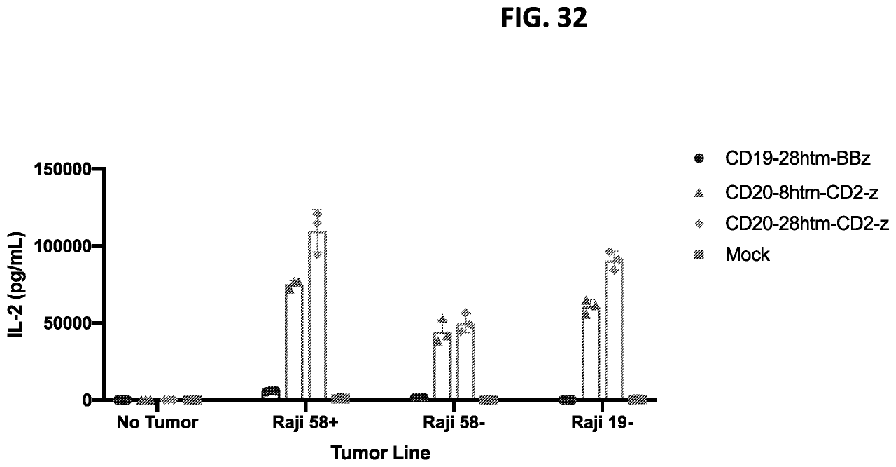

100,000 CAR-T cells were also incubated with Nalm6 CD58⁺ or Nalm6 CD58⁻ cells for 24 hours, and the culture supernatants were tested for IL-2 and IFNγ release by ELISA. As shown in FIG. 10, m971-BBz-CD2 was not effective, but all other CD2-containing CARs (m971-CD2-BBz; m971-CD2-z; and m971-BB-CD2-z) outperformed the conventional m971-BBz CAR against CD58⁻ cells.

Mice (N=5 per group) were inoculated with 1 million luciferase-expressing Nalm6 CD58KO cells. After three days, mice were treated with 3 million MOCK (untransduced), m971-BBz, or m971-CD2-BBz T cells. As shown in FIG. 11, m971-CD2-BBz CAR-T cells demonstrated enhanced tumor control and enhanced survival compared to the conventional m971-BBz CAR.

### Example 7: Construction of CD19 CD2 CARs

This experiment was conducted to compare CD19 CARs with and without CD2 domains against CARs with and without 4-1BB domains.

The following CARs were constructed, using an anti-CD19 scFv and a CD8α transmembrane domain: CD19-BBz (SEQ ID NOs: 65, 66), CD19-CD2-BBz (SEQ ID NOs: 13, 42), CD19-CD2z (SEQ ID NOs: 12, 41), and CD19-BB-CD2z (SEQ ID NOs: 14, 43). These CARs were transduced into T cells as described.

Each of the CAR-Ts (50,000 cells) was incubated with 50,000 Nalm6 tumor cells (GFP⁺, either CD58⁺ or CD58⁻) for 72 hours in an IncuCyte® incubator. As shown in FIG. 12, all of these CAR-T cells performed comparably in terms of cytotoxicity against both CD58⁺ and CD58⁻ cells.

Nalm6 cells are estimated to express about 45,000 CD19 molecules per cell. Normal B cells have been reported to express approximately 22×10³ CD19 molecules per cell, while leukemic B cells express significantly fewer (CLL—13×10³ per cell; mantle cell lymphoma—10×10³ per cell, prior to treatment with a CD19-targeted therapy) (see, e.g., L Ginaldi et al., J Clin Pathol (1998) 51:364-69). Here, Nalm6 cells were engineered to reduce CD19 expression to about 6,196 or 963 CD19 molecules per cell, levels approximating physiological levels found in patients, to study the effect of declining antigen presence in tumor cells.

Each of the CAR-Ts (50,000 cells) was incubated with 50,000 Nalm6(6196) tumor cells (GFP⁺, either CD58⁺ or CD58⁻) for 72 hours in an IncuCyte® incubator. As shown in FIG. 13, CD2 CAR-T outperformed conventional CD19-BBz CAR-T cells in terms of cytotoxicity against CD58⁻ cells. As shown in FIG. 14, these CD2 CARs also outperformed conventional CD19-BBz CAR-T cells in terms of cytokine release against both CD58⁺ and CD58⁻ cells when incubated for 24 hours, as described above.

### Example 8: Choice of Co-Stimulatory Domain in CD2 CARs

This experiment was performed to determine which co-stimulatory domains are useful in CARs that contain a CD2 co-stimulatory domain.

CARs m971-28z (SEQ ID NOs: 61, 62) and m971-CD2-28z (SEQ ID NOs: 19, 48) were prepared and transduced into T cells as described. CAR-T cells (100,000) were co-cultured with an equal number of Nalm6 CD58⁺ or CD58⁻ cells for 24 hours, and the culture supernatant was examined by ELISA for cytokine release. As shown in FIG. 15, adding a CD2 co-stimulatory domain to the CD28 co-stimulatory domain actually decreased IL-2 and IFNγ release significantly. When co-cultured with target cells for 72 in an IncuCyte® and examined for cytotoxicity, conventional m971-28z CAR-T cells performed better than m971-CD2-28z CAR-T cells as shown in FIG. 16.

Conversely, adding CD2 to a 4-1BB CAR improved activity, particularly against CD58KO cells. CARs m971-BBz (SEQ ID NOs: 59, 60), m971-CD2-BBz (SEQ ID NOs: 17, 46), CD19-BBz (SEQ ID NOs: 65, 66), and CD19-CD2-BBz (SEQ ID NOs: 13, 42) were prepared and transduced into T cells as described. CAR-T cells (100,000) were co-cultured with an equal number of Nalm6 CD58⁺ or CD58⁻ cells for 24 hours, and the culture supernatant was examined by ELISA for cytokine release. As shown in FIG. 17, adding a CD2 co-stimulatory domain to the 4-1BB co-stimulatory domain improved the cytokine release against CD58KO cells, and for CD19 CARs adding CD2 improved cytokine release also against CD58⁺ cells. As shown in FIG. 18, when co-cultured with target cells for 72 in an IncuCyte® and examined for cytotoxicity, m971-CD2-BBz CAR-T cells performed better than conventional m971-BBz CAR-T cells, particularly against CD58KO (CD58⁻) cells.

### Example 9: Trans CD2 Chimeric Polypeptides

This experiment was performed to demonstrate trans-CD2 chimeric polypeptide expression and efficacy.

Bicistronic constructs were made for expression of a CAR m971-BBz (having a CD8α transmembrane domain) and a CD2 chimeric polypeptide, separating the coding domains with a 2A sequence (SEQ ID NOs: 69, 70). The 2A sequence used here (P2A) was modified to include a furin cleavage sequence (RKRR) and an EcoRI cleavage site upstream of the P2A sequence, and a XhoI cleavage site downstream. The constructs were as follows: m971-BBz-2A-CD19-CD28tm-CD2 (SEQ ID NOs: 71, 72), m971-BBz-2A-CD19-CD8tm-CD2 (SEQ ID NOs: 73, 74), m971-BBz-2A-CD19-CD2tm-CD2 (SEQ ID NOs: 75, 76), m971-BBz-2A-CD19-CD28tm-stop (control) (SEQ ID NOs: 77, 78), and m971-BBz-2A-stop (control) (SEQ ID NOs: 79, 80). The CD2 chimeric polypeptides thus constructed also had different transmembrane (tm) domains: CD28tm, CD8tm, and CD2tm. FIG. 19 illustrates schematically the difference between an exemplary CAR and a CD2-trans chimeric polypeptide. The left panel depicts a schematic structure for a CD2 CAR, having 4-1BB and CD2 co-stimulatory domains, in addition to a CD3ζ activating domain. The right panel depicts a second generation CAR having a 4-1BB co-stimulatory domain, and a chimeric polypeptide having an antigen binding domain and a CD2 co-stimulatory domain. Note that the chimeric polypeptide lacks a CD3ζ activating domain, and thus acts only in trans to co-stimulate the CAR.

Each of the CAR-Ts (50,000 cells) was incubated with 50,000 Nalm6 tumor cells (GFP⁺, either CD58⁺ or CD58⁻) for 72 hours in an IncuCyte® incubator. As shown in FIG. 20, all CARs performed similarly against CD58⁺ cells, while only m971-BBz-2A-CD19-28tm-CD2 and m971-BBz-2A-CD19-2tm-CD2 performed well against CD58KO cells. Without being bound by any theory, this is believed to be due to interference between the two receptors having substantially the same tm domains.

CAR-T cells (100,000) were co-cultured with an equal number of Nalm6 CD58⁺ or CD58⁻ cells for 24 hours, and the culture supernatant was examined by ELISA for cytokine release. As shown in FIG. 21, CAR-T cells having a chimeric polypeptide in addition to a CAR outperformed CAR-T cells without a chimeric polypeptide in cytokine release against CD58KO cells.

### Example 10: Cis v. Trans CD2 Chimeric Polypeptides

This experiment was performed to compare a trans CAR-chimeric polypeptide combination with CD2 domain-containing cis CARs.

CAR-T cells were prepared with m971-BBz-2A-CD19-28tm-CD2 (SEQ ID NOs: 71, 72), m971-BBz-CD2z (SEQ ID NOs: 18, 47), or m971-CD2z (SEQ ID NOs: 16, 45) constructs. Each group of the CAR-Ts (50,000 cells) was incubated with 50,000 Nalm6 tumor cells (GFP⁺, either CD58⁺ or CD58⁻) for 72 hours in an IncuCyte® incubator. As shown in FIG. 22, the trans construct (m971-BBz-2A-CD19-28tm-CD2) performed better than the two cis constructs.

CAR-T cells (100,000) were co-cultured with an equal number of Nalm6 CD58⁺ or CD58⁻ cells for 24 hours, and the culture supernatant was examined by ELISA for cytokine release. As shown in FIG. 23, the trans construct (m971-BBz-2A-CD19-28tm-CD2) again performed better than the two cis constructs.

### Example 11: Paratope Chimeric Polypeptides

This experiment was performed to study the effect of a chimeric polypeptide targeting a different epitope of the same antigen targeted by a CAR. The scFv's m971 and HA22 each target different epitopes of CD22. In this case, HA22 was used in the chimeric polypeptide constructs, in combination with m971 in the CAR constructs.

CAR-T cells were prepared with m971-BBz-2A-HA22-28tm-CD2 (SEQ ID NOs: 81, 82), m971-BBz-2A-HA22-8tm-CD2 (SEQ ID NOs: 83, 84), m971-BBz-2A-HA22-2tm-CD2 (SEQ ID NOs: 85, 86), m971-BBz-2A-stop (control) (SEQ ID NOs: 79, 80), and m971-BBz-2A-HA22-28tm-stop (control) (SEQ ID NOs: 77, 78). Each group of the CAR-Ts (50,000 cells) was incubated with 50,000 Nalm6 tumor cells (GFP⁺, either CD58⁺ or CD58⁻) for 72 hours in an IncuCyte® incubator. As shown in FIG. 24, each of the trans CAR-chimeric polypeptide combinations performed better than CARs alone.

CAR-T cells (100,000 per group) were co-cultured with an equal number of Nalm6 CD58⁺ or CD58⁻ cells for 24 hours, and the culture supernatant was examined by ELISA for cytokine release. As shown in FIG. 25, m971-BBz-2A-HA22-28tm-CD2 performed somewhat better in cytokine release than m971-BB-CD2z.

### Example 12: Anti-Tumor Activity of CD22 CD2 CAR

This experiment was performed to study the anti-tumor activity of CD22 CAR (m971-BBz) against CD58 KO with or without the CD2 signaling domain.

NSG mice were inoculated with 1 million of the indicated tumor lines which both express luciferase for tracking the tumor by bioluminescence. Three days after inoculation, mice were treated with 3 million MOCK (untransduced) or m971-BBz trans with CD19-TM or CD19-CD2. Tumor BLI was measured 1-2 times weekly.

As shown in FIG. 26, trans CD2 CAR T cells containing m971-BBz CAR (SEQ ID NOs: 59, 60) expressed (co-transduced) alongside a CD2 signaling CAR recognizing CD19 (SEQ ID NOs: 101, 123) (CD22-4-1BBz+CD19-CD2) demonstrated strong anti-tumor activity against CD58KO Nalm6 compared to the traditional m971-BBz CAR (SEQ ID NOs: 59, 60) expressed (co-transduced) alongside a control molecule recognizing CD19 without any signaling domains (SEQ ID NOs: 113, 135) (CD22-4-1BBz+CD19-TM).

### Example 13: In Vitro Trans CAR and CD2 Rescue of CD58 KO

This experiment was performed to study in vitro trans CAR and CD2 rescue of CD58 loss.

CD2 containing receptor recognizing CD20 (with either CD8 or CD28 transmembrane domain) (SEQ ID NO: 109 or 110) was co-expressed with CD22 CAR (m971-BBz; SEQ ID NOs: 59, 60). 100,000 CART cells were cocultured at a 1:1 ratio with Raji or Nalm6 tumor cell line for 24 hours. IL-2 levels were measured in the supernatant by ELISA. As shown in FIG. 27, trans CD22 CAR T cells co-expressed (co-transduced) with CD2 containing receptor recognizing CD20 (m971-BBz+CD20-28tm-CD2 and m971-BBz+CD20-8tm-CD2) were able to rescue CAR T function against CD58 KO cells. This rescue against CD58 loss only occurred when the tumor expressed the target of the CD2 receptor (that is CD20 in this experiment).

CD2 containing receptor recognizing CD20 (with a CD28 hinge-transmembrane domain with or without CD2; SEQ ID NO: 111 or 113) was co-expressed with CD19 CAR (CD19-BBz; SEQ ID NOs: 65, 66). 100,000 CAR T cells were cocultured at a 1:1 ratio with CD58+ and CD58− Raji tumor cell line for 24 hours. IL-2 levels were measured in the supernatant by ELISA. As shown in FIG. 28, trans CD19 CAR T cells co-expressed with CD2 containing receptor recognizing CD20 (CD19-BBz+CD20-28htm-CD2) was able to rescue CAR T cell function against CD58 KO cells.

CAR recognizing CD22 containing CD2 and CD3z endodomain (m971-8tm-CD2-z; SEQ ID NO: 92) was co-expressed trans with CD19-28z CAR (SEQ ID NOs: 63, 64). 100,000 CAR T cells were cocultured at a 1:1 ratio with CD58+ and CD58− Nalm6 tumor cell line for 24 hours. IL-2 levels were measured in the supernatant by ELISA. As shown in FIG. 29, trans CD19-28z and m971-8tm-CD2-z was able to rescue CAR T function against CD58 KO cells and maintained activity against cells that lost either CD19 or CD22 antigen.

CAR recognizing C20 containing CD2 and CD3z endodomain (CD20-IgG1long-28htm-CD2-z; SEQ ID NOs: 111, 133) was co-expressed (co-transduced) trans with CD19-BBz CAR (SEQ ID NOs: 65, 66). 100,000 CAR T cells were cocultured at a 1:1 ratio with CD58+ and CD58− Raji tumor cell line for 24 hours. IL-2 levels were measured in the supernatant by ELISA. As shown in FIG. 30, trans CD19-BBz (SEQ ID NOs: 65, 66) and CD20-28htm-CD2-z (CD20-IgG1long-28htm-CD2-z; SEQ ID NOs: 111, 133) was able to rescue CAR T cell function against CD58 KO cells and maintained activity against cells that lost the target antigen such as CD19. A trans CD19-BBz and CD20-28htm control receptor (SEQ ID NOs: 113, 135) was not able to rescue function.

### Example 14: In Vivo Trans CAR and CD2 Rescue of CD58 KO

This experiment was performed to study in vivo trans CAR and CD2 rescue of CD58 loss.

NSG mice were inoculated with 1 million of the indicated tumor lines which both express luciferase for tracking the tumor by bioluminescence. Three days after tumor inoculation, mice were treated with 3 million CAR T cells as indicated in FIG. 31. Tumor bioluminesence was measured 1-2 times weekly. N=5 mice per group. As shown in FIG. 31, CD19 CAR or CD22 CAR cells integrating CD2 signaling in trans overcame CD58 loss. In this experiment, the trans CD2 containing receptors also integrated CD3zeta (CD19-28tm-CD2-z; SEQ ID NOs: 98, 118) so that they could rescue CAR T cell function against CD58KO cells and also maintain activity against the cells that lost the target antigen such as CD19.

### Example 15: CD20 CD2 CAR Rescue of CD58 and CD19 Loss

This experiment was performed to compare the effect of CD20 CD2 CAR in CD58 loss and CD19 loss.

100,000 CAR T cells of three CARs (CD19-28htm-BBz; CD20-8htm-CD2-z; CD20-28htm-CD2-z) were co-cultured at a 1:1 ratio with Raji tumor cell line (CD58+; CD58−; CD19−) for 24 hours. IL-2 levels were measured in the supernatant by ELISA. As shown in FIG. 32, a CD20 targeted CAR that integrated CD2 signaling was able to overcome both loss of CD58 and loss of CD19, which are both common mechanisms of immune escape from CAR T cell therapy.

### Example 16: Enhancing CD2 Signaling

CD2 signaling in a T cell expressing a transgenic TCR or in bulk tumor infiltrating lymphocytes (TILs) grown ex vivo can be enhanced by several methods. The TILs can then be given back to a patient.

For example, the T cells can be transduced with a co-receptor that enhances CD2 signaling (in addition to expressing the transgenic TCR). The co-receptor comprising an extracellular ligand binding domain, a transmembrane domain, and a CD2 signaling domain can be transcribed in a virus or other vector and can provide CD2 signaling in trans even when the target tumor cells express low, absent, or mutated CD58. The extracellular portion of the co-receptor can comprise an scFv recognizing an antigen expressed by the tumor cells or a ligand for a common receptor expressed on the target tumor cell of interest.

Another way to enhance CD2 signaling in a CAR T cell, a transgenic TCR T cell, or bulk TILs can comprise transducing the T cells to constitutively express a secreted molecule capable of crosslinking the cell's native CD2 through use of one or more anti-CD2 scFv's, antibodies, Fabs, DARPINs, ligands, or other binders/antigen binding domains. Alternatively, the secreted molecule can be expressed under an activation switch. The secreted molecule can be membrane bound and can consist of two scFv's connected by a linker: one scFv that binds CD2 on the T cell (activating its native CD2 signaling) and the other scFv or ligand recognizing a protein or target expressed on tumor cells such that CD2 is crosslinked and activated when the T cell encounters tumor cells.

1-151. (canceled)

152. A composition comprising a chimeric polypeptide comprising, in an order from N-terminal to C terminal:
an antigen binding domain,
a transmembrane domain, and
a cytoplasmic signaling domain;
wherein the cytoplasmic signaling domain comprises a CD2 signaling domain; wherein the cytoplasmic signaling domain lacks a CD3-zeta activating domain.

153. The composition of claim 152, wherein the cytoplasmic signaling domain consists of the CD2 signaling domain.

154. The composition of claim 152, wherein the CD2 signaling domain comprises an amino acid sequence with at least 80% identity to the amino acid sequence of SEQ ID NO: 8, or (ii) consists of the amino acid sequence of SEQ ID NO: 8.

155. The composition of claim 152, wherein the cytoplasmic signaling domain further comprises a co-stimulating domain selected from a 4-1BB signaling domain, a CD27 signaling domain, an OX40 signaling domain, a CD28 signaling domain, a CD278 signaling domain, a CD40 signaling domain, a CD40L signaling domain, or a toll-like receptor signaling domain, or any combination thereof.

156. The composition of claim 152, wherein the antigen binding domain is specific for a B cell surface antigen or a tumor associated antigen.

157. The composition of claim 156, wherein the antigen binding domain is specific for CD19, CD20, or CD22.

158. The composition of claim 152, wherein the antigen binding domain comprises an scFv.

159. The composition of claim 152, wherein the antigen binding domain comprises an amino acid sequence with at least 80% identity to a sequence of SEQ ID NO: 1, 2, 3 or 4.

160. The composition of claim 152, wherein the transmembrane domain is selected from the group consisting of all or part of a transmembrane domain of CD3ζ, CD2, CD8α, CD28, CD40, CTLA4, OX40, PD-1, 4-1BB (CD137), FcERIγ, ICOS (CD278), ILRB (CD122), and IL-2RG (CD132).

161. The composition of claim 160, wherein the transmembrane domain comprises an amino acid sequence with at least 95% identity to a sequence of SEQ ID NO: 5, 6, or 7.

162. The composition of claim 152, wherein the chimeric polypeptide comprises an amino acid sequence with at least 80% identity to any one of the sequences of SEQ ID NOs: 20-29 and 123-132.

163. The composition of claim 152, wherein the antigen binding domain is specific for CD19, CD20, or CD22, and wherein the transmembrane domain is a CD28 transmembrane domain, CD2 transmembrane domain, or a CD8 transmembrane domain.

164. The composition of claim 152, further comprising an additional polypeptide, wherein the additional polypeptide comprises a chimeric antigen receptor (CAR), a T cell receptor (TCR), or a TCR-CAR.

165. The composition of claim 164, wherein the additional polypeptide comprises the CAR, and wherein the CAR comprises a CD3-zeta activating domain.

166. The composition of claim 164, wherein the additional polypeptide comprises an antigen binding domain, and wherein the antigen binding domain is specific for a B cell surface antigen or a tumor associated antigen.

167. The composition of claim 166, wherein the antigen binding domain of the additional polypeptide and the antigen binding domain of the chimeric polypeptide are specific for different antigens.

168. The composition of claim 167, wherein the antigen binding domain of the additional polypeptide or the antigen binding domain of the chimeric polypeptide is CD19, CD20, or CD22.

169. The composition of claim 167, wherein the composition comprises the CAR, wherein the antigen binding domain of the CAR is specific for CD22, and wherein the antigen binding domain of the chimeric polypeptide is specific for CD19.

170. The composition of claim 169, wherein the antigen binding domain of the CAR comprises a scFv specific for CD22, and wherein the scFv specific for CD22 comprises an amino acid sequence with at least 80% identity to a sequence of SEQ ID NO: 2; and wherein the antigen binding domain of the chimeric polypeptide comprises a scFv specific for CD19, and wherein the scFv specific for CD19 comprises an amino acid sequence with at least 80% identity to a sequence of SEQ ID NO: 1.

171. A nucleic acid comprising a sequence encoding the chimeric polypeptide of the composition of claim 152.

172. A cell, comprising the composition of claim 152 or a nucleic acid encoding the chimeric polypeptide of the composition.

173. The cell of claim 172, wherein the cell is autologous.

174. The cell of claim 172, wherein the immune cell is a T cell.

175. A method treating of a subject having a hyperproliferative disorder, the method comprising: administering to the subject a therapeutically effective amount of the cell of claim 172.

176. The method of claim 175, wherein the hyperproliferative disorder is characterized by proliferation of a target cell that (i) lacks expression of CD58; (ii) expresses a reduced level of CD58; or (iii) expresses a form of CD58 that has reduced ability to activate a CD2.

177. A chimeric antigen receptor (CAR), comprising:
an antigen binding domain;
a transmembrane domain; and
a cytoplasmic signaling domain, wherein the cytoplasmic signaling domain comprises a CD2 signaling domain, an activating domain, and a co-stimulating signaling domain;
wherein the co-stimulating signaling domain is not a CD28 signaling domain.

178. A transgenic T cell receptor (TCR), comprising an antigen binding domain and a transmembrane domain, a cytoplasmic signaling domain, wherein the cytoplasmic signaling domain comprises a CD2 signaling domain.
